(19) United States
(12) Patent Application Publication
(10) Pub. No.: US 2013/0072519 A1
(43) Pub. Date: Mar. 21, 2013

(54) 2-PHENYL BENZOYLAMIDES

(71) Applicants: Edward Lee Conn, Griswold, CT (US); David Hepworth, Concord, MA (US); Yingmei Qi, New London, CT (US); Benjamin Neil Rocke, Manchester, CT (US); Roger Benjamin Ruggeri, Waterford, CT (US); Yan Zhang, Falmouth, ME (US)

(72) Inventors: Edward Lee Conn, Griswold, CT (US); David Hepworth, Concord, MA (US); Yingmei Qi, New London, CT (US); Benjamin Neil Rocke, Manchester, CT (US); Roger Benjamin Ruggeri, Waterford, CT (US); Yan Zhang, Falmouth, ME (US)

(21) Appl. No.: 13/699,230

(22) PCT Filed: May 9, 2011
(86) PCT No.: PCT/IB2011/052037

§ 371 Date: Nov. 20, 2012

Related U.S. Application Data
(60) Provisional application No. 61/347,110, filed on May 21, 2010.

(51) Int. Cl. C07D 307/83; A61K 31/4035; A61K 31/435; C07C 235/84; A61K 31/216; C07D 221/04; C07D 209/44; A61K 31/343

(52) U.S. Cl. CPC C07D 307/83; C07D 209/44; A61K 31/4035; A61K 31/343; C07C 235/84; A61K 31/216; C07D 221/04; A61K 31/435
USPC 514/299; 548/472; 514/416; 549/305; 514/470; 560/41; 514/533; 546/112

(57) ABSTRACT

Compounds of Formula I that inhibit microsomal triglyceride transfer protein (MTP) and/or apolipoprotein B (Apo B) secretion and their uses in the treatment of diseases linked thereto in animals are described herein.

This application is a 371 application of PCT/IB2011/052037, filed May 9, 2011, which claims the benefit of U.S. Provisional Application No. 61/347,110, filed May 21, 2010, hereby incorporated by reference in their entireties.

### FIELD OF THE INVENTION

The present invention relates to a new class of 2-phenyl benzoylamide compounds, pharmaceutical compositions containing these compounds, and their use to inhibit microsomal triglyceride transfer protein (MTP) and/or apolipoprotein B (Apo B) secretion.

### BACKGROUND

Diabetes mellitus are disorders in which high levels of blood glucose occur as a consequence of abnormal glucose homeostasis. The most common forms of diabetes mellitus are Type I (also referred to as insulin-dependent diabetes mellitus) and Type II diabetes (also referred to as non-insulin-dependent diabetes mellitus). Type II diabetes, accounting for roughly 90% of all diabetic cases, is a serious progressive disease that results in microvascular complications (including retinopathy, neuropathy and nephropathy) as well as macrovascular complications (including accelerated atherosclerosis, coronary heart disease and stroke).

Currently, there is no cure for diabetes. Standard treatments for the disease are limited, and focus on controlling blood glucose levels to minimize or delay complications. Current treatments target either insulin resistance (metformin, thiazolidinediones), or insulin release from beta cells (sulphonylureas, exanatide). Sulphonylureas and other compounds that act via depolarization of the beta cell promote hypoglycemia as they stimulate insulin secretion independent of circulating glucose concentrations. One approved drug, exanatide, stimulates insulin secretion only in the presence of high glucose, but must be injected due to a lack of oral bioavailablity. Sitagliptin, a dipeptidyl peptidase IV inhibitor, is a new drug that increases blood levels of incretin hormones, which can increase insulin secretion, reduce glucagon secretion and have other less well characterized effects. However, sitagliptin and other dipeptidyl peptidases IV inhibitors may also influence the tissue levels of other hormones and peptides, and the long-term consequences of this broader effect have not been fully investigated.

Thus, there has been great interest in the discovery of agents that treat diabetes. It is well known that metabolic diseases have negative effects on other physiological systems and there is often co-occurrence of multiple disease states (e.g. Type I diabetes, Type II diabetes, inadequate glucose tolerance, insulin resistance, hyperglycemia, hyperlipidemia, hypertriglyceridemia, hypercholesterolemia, dyslipidemia, obesity or cardiovascular disease in “Syndrome X”) or secondary diseases which occur secondary to diabetes such as kidney disease, and peripheral neuropathy.

Microsomal triglyceride transfer protein catalyzes the transport of triglyceride, cholesteryl ester, and phospholipids and has been implicated as a putative mediator in the assembly of Apo B-containing lipoproteins, which are biomolecules that contribute to the formation of atherosclerotic lesions. Specifically, the subcellular (lumen of the microsomal fraction) and tissue (liver and intestine) distribution of MTP have led to speculation that it plays a role in the assembly of plasma lipoproteins, as these are the sites of plasma lipoprotein assembly. The ability of MTP to catalyze the transport of triglyceride between membranes is consistent with this speculation, and suggests that MTP may catalyze the transport of triglyceride from its site of synthesis in the endoplasmic reticulum membrane to nascent lipoprotein particles within the lumen of the endoplasmic reticulum.

Compounds which inhibit MTP and/or otherwise inhibit Apo B secretion are accordingly useful in the treatment of atherosclerosis and conditions frequently associated therewith. Such conditions include, for example, hypercholesterolemia, hypertriglyceridemia, pancreatitis, diabetes, and obesity. For a detailed discussion, see for example, Wetterau et al., Science, 258, 999-1001, (1992), Wetterau et al., Biochem. Biophys. Acta., 875, 610-617 (1986). Moreover, MTP inhibitors developed in the past, although useful in treating a variety of cardiovascular and metabolic diseases and conditions, have not only inhibited MTP activity in the small intestine, but also in the liver. This may lead to fatty liver disease and possible hepatotoxicity. Thus, treatment of the diabetic condition should be of benefit to such interconnected disease states.

### SUMMARY OF THE INVENTION

The present invention is directed at compounds having the Formula I

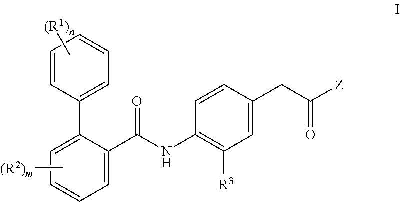

wherein:

R¹ is, (C₁-C₆)alkyl, (C₃-C₇)cycloalkyl, (C₁-C₆)alkoxy, —C(O)—OH, hydroxyl, or halo, wherein each alkyl and alkoxy is optionally substituted with one or more hydroxyl, halo or oxy, and n is 0, 1 or 2;

R² is (C₁-C₆)alkyl, (C₃-C₇)cycloalkyl, (C₁-C₆)alkoxy, —O(O)—OH, hydroxyl, or halo wherein each alkyl and alkoxy is optionally substituted with one or more hydroxyl, halo or oxy, and m is 0, 1 or 2;

R³ is hydrogen, (C₁-C₆)alkyl, halo, or —C(O)—N—R^{4a}R^{4b};

R^{4a} and R^{4b} are each independently hydrogen, (C₁-C₆)alkyl, (C₁-C₆)alkoxy, (C₃-C₇)cycloalkyl, or R^{4a} and R^{4b} are taken together with the N to which they are attached to form a 4 to 7-membered heterocycle optionally substituted with (C₁-C₆)alkyl;

Z is —O—R⁵;

R⁵ is

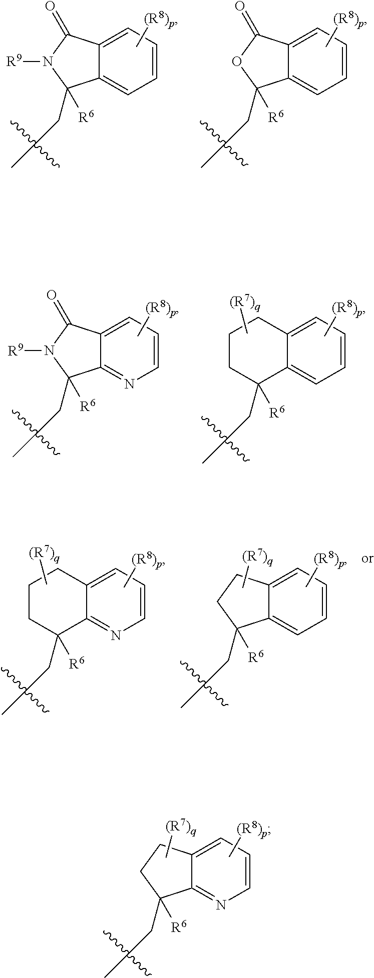

R⁶ is —C(O)—O—(C₁-C₆)alkyl, —C(O)—O—(C₁-C₆)alkyl-aryl, or —O(O)—OH;

R⁷ is hydrogen, hydroxyl, oxo, (C₁-C₆)alkyl, or (C₁-C₆)alkoxy, wherein each alkyl and alkoxy is optionally substituted with hydroxyl, halo or oxy, and q is 0, 1 or 2;

R⁸ is (C₁-C₆)alkyl, (C₃-C₇)cycloalkyl, (C₁-C₆)alkoxy, or halo, wherein each alkyl and alkoxy is optionally substituted with hydroxyl, halo or oxy, and p is 0, 1 or 2; and

R⁹ is hydrogen, (C₁-C₆)alkyl, (C₁-C₆)alkoxy, (C₃-C₇)cycloalkyl, aryl, or aralkyl wherein each alkyl and alkoxy is optionally substituted with hydroxyl, halo or oxy, and each aryl and aralkyl are optionally substituted with (C₁-C₆)alkyl, (C₁-C₆)alkoxy, hydroxyl, halo pr oxy;

or a pharmaceutically acceptable salt thereof.

Furthermore, the application is directed at the following compounds:
Ethyl (1R)-1-({2-[3-(Dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate;
Ethyl (1R)-1-({2-[3-(dimethylcarbamoyl)-4-{[(4′-isopropoxybiphenyl-2-yl)carbonyl]amino}phenyl]acetoxy}methyl)-2-methyl-3-oxoisoindoline-1-carboxylate;
Ethyl 1-({2-[3-(dimethylcarbamoyl)-4-({[5-methyl-4′-(trifluoromethyl)biphenyl-2-yl]carbonyl}amino)phenyl]acetoxy}methyl)-2-methyl-3-oxoisoindoline-1-carboxylate;
Ethyl 7-({2-[3-(dimethylcarbamoyl)-4-({[5-methyl-4′-(trifluoromethyl)biphenyl-2-yl]carbonyl}amino)phenyl]acetoxy}methyl)-6,7-dihydro-5H-cyclopenta[b]pyridine-7-carboxylate;
Ethyl 7-({2-[3-(dimethylcarbamoyl)-4-({[6-methyl-4′-(trifluoromethyl)biphenyl-2-yl]carbonyl}amino)phenyl]acetoxy}methyl)-6,7-dihydro-5H-cyclopenta[b]pyridine-7-carboxylate:
Ethyl (1R)-1-({2-[3-(dimethylcarbamoyl)-4-({[5-methoxy-4′-(trifluoromethyl)biphenyl-2-yl]carbonyl}amino)phenyl]acetoxy}methyl)-2-methyl-3-oxoisoindoline-1-carboxylate;
Ethyl (1R)-1-({2-[3-(dimethylcarbamoyl)-4-({[6-methoxy-4′-(trifluoromethyl)biphenyl-2-yl]carbonyl}amino)phenyl]acetoxy}methyl)-2-methyl-3-oxoisoindoline-1-carboxylate;
Ethyl (1R)-1-({2-[3-(dimethylcarbamoyl)-4-{[(4′-isopropoxy-5-methylbiphenyl-2-yl)carbonyl]amino}phenyl]acetoxy}methyl)-2-methyl-3-oxoisoindoline-1-carboxylate;
Ethyl 7-({2-[3-(dimethylcarbamoyl)-4-{[(4′-isopropoxybiphenyl-2-yl]carbonyl}amino)phenyl]acetoxy}methyl)-6,7-dihydro-5H-cyclopenta[b]pyridine-7-carboxylate;
Ethyl 7-({2-[3-(dimethylcarbamoyl)-4-({[6-methoxy-4′-(trifluoromethyl)biphenyl-2-yl]carbonyl}amino)phenyl]acetoxy}methyl)-6,7-dihydro-5H-cyclopenta[b]pyridine-7-carboxylate
[3-dimethylcarbamoyl-4-{[(6-methyl-4′-trifluoromethylbiphenyl-2-yl)carbonyl]amino}phenyl]acetate;
[3-dimethylcarbamoyl-4-{[(4′-isopropoxybiphenyl-2-yl)carbonyl]amino}phenyl]acetate;
[3-dimethylcarbamoyl-4-{[(5-methyl-4′-trifluoromethylbiphenyl-2-yl)carbonyl]amino}phenyl]acetate;
[3-dimethylcarbamoyl-4-{[(5-methoxy-4′-trifluoromethylbiphenyl-2-yl)carbonyl]amino}phenyl]acetate;
[3-dimethylcarbamoyl-4-{[(6-methoxy-4′-trifluoromethylbiphenyl-2-yl)carbonyl]amino}phenyl]acetate;
[3-dimethylcarbamoyl-4-{[(5-methyl-4′-isopropoxybiphenyl-2-yl)carbonyl]amino}phenyl]acetate; and
[3-dimethylcarbamoyl-4-{[(6-methoxy-4′-trifluoromethylbiphenyl-2-yl)carbonyl]amino}phenyl]acetate;

or a pharmaceutically acceptable salt thereof.

The compounds of Formula I inhibit microsomal triglyceride transfer protein (MTP) and/or apolipoprotein B (Apo B) secretion. As such, said compounds are useful for the treatment of diseases and conditions including Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, pancreatitis, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance. The compounds may be used to treat neurological disorders such as Alzheimer's, schizophrenia, and impaired cognition. The compounds will also be beneficial in gastrointestinal illnesses such as inflammatory bowel disease, ulcerative colitis, Crohn's disease, irritable bowel syndrome, etc. As noted above the compounds may also be used to stimulate weight loss in obese patients, especially those afflicted with diabetes.

A further embodiment of the invention is directed to pharmaceutical compositions containing a compound of Formula I. Such formulations will typically contain a compound of Formula I in admixture with at least one pharmaceutically acceptable excipient. Such formulations may also contain at least one additional pharmaceutical agent. Examples of such agents include anti-obesity agents, anti-diabetic agents, anti-hyperglycemic agents, lipid lowering agents, and anti-hypertensive agents. Additional aspects of the invention relate to the use of the compounds of Formula I in the preparation of medicaments for the treatment of diabetes and related conditions as described herein.

It is to be understood that both the foregoing summary and the following detailed description are exemplary and explanatory only and are not restrictive of the invention, as claimed.

### DETAILED DESCRIPTION OF THE INVENTION

The present invention may be understood more readily by reference to the following detailed description of exemplary embodiments of the invention and the examples included therein.

It is to be understood that this invention is not limited to specific synthetic methods of making that may of course vary. It is also to be understood that the terminology used herein is for the purpose of describing particular embodiments only and is not intended to be limiting. In this specification and in the claims that follow, reference will be made to a number of terms that shall be defined to have the following meanings:

As used herein in the specification, “a” or “an” may mean one or more. As used herein in the claim(s), when used in conjunction with the word “comprising”, the words “a” or “an” may mean one or more than one. As used herein “another” may mean at least a second or more.

The term “about” refers to a relative term denoting an approximation of plus or minus 10% of the nominal value it refers, in one embodiment, to plus or minus 5%, in another embodiment, to plus or minus 2%. For the field of this disclosure, this level of approximation is appropriate unless the value is specifically stated require a tighter range.

“Compounds” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. By “tautomers” is meant chemical compounds that may exist in two or more forms of different structure (isomers) in equilibrium, the forms differing, usually, in the position of a hydrogen atom. Various types of tautomerism can occur, including keto-enol, ring-chain and ring-ring tautomerism. The expression “prodrug” refers to compounds that are drug precursors which following administration, release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the compounds of the present invention include but are not limited to those having a carboxyl moiety wherein the free hydrogen is replaced by (C₁-C₄)alkyl, (C₂-C₇)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

The following paragraphs describe exemplary ring(s) for the generic ring descriptions contained herein.

By “halo” or “halogen” is meant chloro, bromo, iodo, or fluoro.

By “alkyl” is meant straight chain saturated hydrocarbon or branched chain saturated hydrocarbon. Exemplary of such alkyl groups (assuming the designated length encompasses the particular example) are methyl, ethyl, propyl, isopropyl, butyl, sec-butyl, tertiary butyl, isobutyl, pentyl, isopentyl, neopentyl, tertiary pentyl, 1-methylbutyl, 2-methylbutyl, 3-methylbutyl, hexyl, isohexyl, heptyl and octyl.

By “alkoxy” is meant straight chain saturated alkyl or branched chain saturated alkyl bonded through an oxy. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy.

“Aralkyl” means an alkyl group with an aryl group substituting for a hydrogen atom of the alkyl group.

The term “aryl” means a carbocyclic aromatic system containing one, two or three rings wherein such rings may be fused. If the rings are fused, one of the rings must be fully unsaturated and the fused ring(s) may be fully saturated, partially unsaturated or fully unsaturated. The term “fused” means that a second ring is present (ie, attached or formed) by having two adjacent atoms in common (ie, shared) with the first ring. The term “fused” is equivalent to the term “condensed”. The term “aryl” embraces aromatic radicals such as phenyl, naphthyl, tetrahydronaphthyl, indane and biphenyl.

“Cycloalkyl” refers to a nonaromatic ring that is fully hydrogenated and exists as a single ring. Examples of such carbocyclic rings include cyclopropyl, cyclobutyl, cyclopentyl, and cyclohexyl.

The term “heterocycle” means a nonaromatic carbocyclic system containing one, two, three or four heteroatoms selected independently from oxygen, nitrogen and sulfur and having one, two or three rings wherein such rings may be fused, wherein fused is defined above. The term “heterocycle” includes but is not limited to lactones, lactams, cyclic ethers and cyclic amines, including the following exemplary ring systems: epoxide, tetrahydrofuran, tetrahydropyran, dioxane, aziridines, pyrrolidine, piperidine, and morpholine.

It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3- or 4-pyridyl, the term “thienyl” means 2- or 3-thienyl, and so forth.

“Patient” refers to warm blooded animals such as, for example, guinea pigs, mice, rats, gerbils, cats, rabbits, dogs, cattle, goats, sheep, horses, monkeys, chimpanzees, and humans.

By “pharmaceutically acceptable” is meant that the substance or composition must be compatible chemically and/or toxicologically, with the other ingredients comprising a formulation, and/or the mammal being treated therewith.

As used herein, the expressions “reaction-inert solvent” and “inert solvent” refer to a solvent or a mixture thereof which does not interact with starting materials, reagents, intermediates or products in a manner which adversely affects the yield of the desired product.

As used herein, the term “selectivity” or “selective” refers to a greater effect of a compound in a first assay, compared to the effect of the same compound in a second assay. For example, in “gut selective” compounds, the first assay is for the half life of the compound in the intestine and the second assay is for the half life of the compound in the liver.

“Therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

The present invention also relates to the pharmaceutically acceptable acid addition salts of compounds of the present invention. The acids which are used to prepare the pharmaceutically acceptable acid addition salts of the aforementioned base compounds of this invention are those which form non-toxic acid addition salts, (i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The invention also relates to base addition salts of the compounds of the present invention. The chemical bases that may be used as reagents to prepare pharmaceutically acceptable base salts of those compounds of the present invention that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

The chemist of ordinary skill will recognize that certain compounds of this invention will contain one or more atoms which may be in a particular stereochemical or geometric configuration, giving rise to stereoisomers and configurational isomers. All such isomers and mixtures thereof are included in this invention. Hydrates and solvates of the compounds of this invention are also included.

Where the compounds of the present invention possess two or more stereogenic centers and the absolute or relative stereochemistry is given in the name, the designations R and S refer respectively to each stereogenic center in ascending numerical order (1, 2, 3, etc.) according to the conventional IUPAC number schemes for each molecule. Where the compounds of the present invention possess one or more stereogenic centers and no stereochemistry is given in the name or structure, it is understood that the name or structure is intended to encompass all forms of the compound, including the racemic form. Names for the compounds were generated using the software ACD Labs Name Software v7.11.

The compounds of this invention may contain olefin-like double bonds. When such bonds are present, the compounds of the invention exist as cis and trans configurations and as mixtures thereof. The term “cis” refers to the orientation of two substituents with reference to each other and the plane of the ring (either both “up” or both “down”). Analogously, the term “trans” refers to the orientation of two substituents with reference to each other and the plane of the ring (the substituents being on opposite sides of the ring).

Alpha and Beta refer to the orientation of a substituent with reference to the plane of the ring. Beta is above the plane of the ring and Alpha is below the plane of the ring.

This invention also includes isotopically-labeled compounds, which are identical to those described by formula I, except for the fact that one or more atoms are replaced by one or more atoms having specific atomic mass or mass numbers. Examples of isotopes that can be incorporated into compounds of the invention include isotopes of hydrogen, carbon, nitrogen, oxygen, sulfur, fluorine, and chlorine such as ²H, ³H, ¹³C, ¹⁴C, ¹⁵N, ¹⁸O, ¹⁷O, ¹⁸F, and ³⁶Cl respectively. Compounds of the present invention, prodrugs thereof, and pharmaceutically acceptable salts of the compounds or of the prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., ²H), can afford certain therapeutic advantages resulting from greater metabolic stability, for example increased in vivo half-life or reduced dosage requirements and, hence, may be preferred in some circumstances. Isotopically labeled compounds of this invention and prodrugs thereof can generally be prepared by carrying out the procedures disclosed in the schemes and/or in the Examples below, by substituting a readily available isotopically labeled reagent for a non-isotopically labeled reagent.

In one embodiment, R³ is —C(O)—N—R^{4a}R^{4b}.

In another embodiment, q is 0.

In another embodiment, R⁵ is

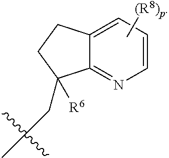

In another embodiment, R⁵ is

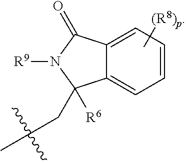

In another embodiment, p is 0.

In another embodiment, R⁹ is hydrogen or (C₁-C₃)alkyl.

In another embodiment, R⁶ is —C(O)—O—(C₁-C₆)alkyl.

In another embodiment, m and n are each independently 0 or 1 and R¹ and R² are each independently (C₁-C₃)alkyl, (C₁-C₃)alkoxy or trifluoromethyl.

In another embodiment, the method for treating a disease includes the administration of a therapeutically effective amount of a compound according to the invention to a patient in need thereof, wherein the disease, condition or disorder is selected from Type I diabetes, Type II diabetes mellitus, idiopathic type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, pancreatitis, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance, hyper apo B lipoproteinemia, Alzheimer's disease, schizophrenia, impaired cognition, inflammatory bowel disease, ulcerative colitis, Crohn's disease, and irritable bowel syndrome.

In another embodiment, the pharmaceutical composition a compound of this invention present in a therapeutically effective amount, in admixture with at least one pharmaceutically acceptable excipient. In another embodiment, the pharmaceutical composition includes at least one additional pharmaceutical agent selected from the group consisting of an anti-obesity agent, an anti-diabetic agent, an anti-hyperglycemic agent, a lipid lowering agent, and an anti-hypertensive agent. In another embodiment, the compound of this invention and additional pharmaceutical agents are administered simultaneously. In yet another embodiment, the compound of this invention and additional pharmaceutical agents are administered sequentially in any order.

Lipid lowering agents include lipase inhibitors, NPY receptor antagonists, LDL-cholesterol lowering agents, triglyceride lowering agents, HMG-CoA reductase inhibitors, cholesterol synthesis inhibitors, cholesterol absorption inhibitors, CETP inhibitors, PPAR modulators or other cholesterol lowering agents such as a fibrate, niacin, an ion-exchange resin, an antioxidant, an ACAT inhibitor or a bile acid sequestrant. Other pharmaceutical agents useful in the practice of the combination aspect of the invention include bile acid reuptake inhibitors, ileal bile acid transporter inhibitors, ACC inhibitors, antihypertensive agents (such as Norvasc®), antibiotics, antidiabetics (such as metformin), PPAR.gamma. activators, sulfonylureas, insulin, aldose reductase inhibitors (AR.sup.1) (e.g., zopolrestat), sorbitol dehydrogenase inhibitors (SDI)), and anti-inflammatory agents such as aspirin or, preferably, an anti-inflammatory agent that inhibits cyclooxygenase-2 (Cox-2) to a greater extent than it inhibits cyclooxygenase-1 (Cox-1) such as celecoxib (U.S. Pat. No. 5,466,823), valdecoxib (U.S. Pat. No. 5,633,272, parecoxib (U.S. Pat. No. 5,932,598), deracoxib (CAS RN 169590-41-4), etoricoxib (CAS RN 202409-33-4) or lumiracoxib (CAS RN 220991-20-8).

Lipase inhibitors are useful in the practice of the combination aspect of the present invention. Lipase inhibitors inhibit the metabolic cleavage of dietary triglycerides into free fatty acids and monoglycerides. Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a monoglyceride and a fatty acid. The resultant free fatty acids and monoglycerides are incorporated into bile acid-phospholipid micelles, which are subsequently absorbed at the level of the brush border of the small intestine. The micelles eventually enter the peripheral circulation as chylomicrons. Lipase inhibition activity is readily determined by the use of standard assays well known in the art. See, for example, Methods Enzymol. 286: 190-231, incorporated herein by reference.

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors of this lipase find utility in the treatment of obesity and associated conditions.

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams, et al., Gastroenterology, 92, 125 (1987).

A variety of pancreatic lipase inhibitors useful in the present invention are described hereinbelow. The pancreatic lipase inhibitors lipstatin, (2S,3S,5S,7Z,10Z)-5-[(S)-2-formamido-4-methyl-valeryloxy]-2-hexyl-3-hydroxy-7,10-hexadecanoic acid lactone, and tetrahydrolipstatin, (2S,3S,5S)-5-[(S)-2-formamido-4-methyl-valeryloxy]-2-hexyl-3-hydroxy-hexadecanoic 1,3 acid lactone, and the variously substituted N-formylleucine derivatives and stereoisomers thereof, are disclosed in U.S. Pat. No. 4,598,089. Tetrahydrolipstatin may be prepared as described in U.S. Pat. Nos. 5,274,143; 5,420,305; 5,540,917; and 5,643,874. The pancreatic lipase inhibitor FL-386, 1-[4-(2-methylpropyl)cyclohexyl]-2-[(phenylsulfonyl)oxy]-ethanone, and variously substituted sulfonate derivatives related thereto are disclosed in U.S. Pat. No. 4,452,813. The pancreatic lipase inhibitor WAY-121898, which is 4-phenoxyphenyl-4-methylpiperidin-1-yl-carboxylate, and various carbamate esters and pharmaceutically acceptable salts related thereto are disclosed in U.S. Pat. Nos. 5,512,565; 5,391,571 and 5,602,151. The pancreatic lipase inhibitor valilactone and a process for preparing it by microbial cultivation of Actinomycetes strain MG147-CF2 are disclosed in Kitahara, et al., J. Antibiotics, 40 (11), 1647-1650 (1987). The pancreatic lipase inhibitors ebelactone A and ebelactone B and processes for preparing them by microbial cultivation of Actinomycetes strain MG7-G1 are disclosed in Umezawa, et al., J. Antibiotics, 33, 1594-1596 (1980). The use of ebelactones A and B in the suppression of monoglyceride formation is disclosed in Japanese Kokai 08-143457, published Jun. 4, 1996. All of the references cited above are incorporated herein by reference.

Preferred lipase inhibitors include lipstatin, tetrahydrolipstatin, valilactone, esterastin, ebelactone A, and ebelactone B, particularly tetrahydrolipstatin. The lipase inhibitor N-3-trifluoromethylphenyl-N′-3-chloro-4′-trifluoromethylphenylurea, and the various urea derivatives related thereto are disclosed in U.S. Pat. No. 4,405,644. Esteracin is disclosed in U.S. Pat. Nos. 4,189,438 and 4,242,453. The lipase inhibitor cyclo-O,O′-[(1,6-hexanediyl)-bis-(iminocarbonyl)]dioxime and the various bis(iminocarbonyl)dioximes related thereto may be prepared as described in Petersen et al., Liebig's Annalen, 562, 205-229 (1949). All of the references cited above are incorporated herein by reference.

Preferred NPY receptor antagonists include NPY Y5 receptor antagonists, such as the spiro compounds described in U.S. Pat. Nos. 6,566,367; 6,649,624; 6,638,942; 6,605,720; 6,495,559; 6,462,053; 6,388,077; 6,335,345 and 6,326,375; U.S. patent application publication Nos. 2002/0151456 and 2003/036652 and PCT patent application publication Nos. WO 03/010175; WO 03/082190 and WO 02/048152.

A slow-release form of niacin is commercially available under the brand name Niaspan. Niacin may also be combined with other therapeutic agents such as lovastatin, which is an HMG-CoA reductase inhibitor. This combination therapy is known as Advicor® (Kos Pharmaceuticals Inc.

Any HMG-CoA reductase inhibitor may be used as the second compound in the combination aspect of this invention. The term HMG-CoA reductase inhibitor refers to compounds that inhibit the bioconversion of hydroxymethylglutaryl-coenzyme A to mevalonic acid catalyzed by the enzyme HMG-CoA reductase. Assays for determining are known in the art (e.g., Meth. Enzymol. 1981; 71:455-509 and references cited therein). HMG-CoA reductase inhibitors of interest herein include those disclosed in U.S. Pat. No. 4,231,938 (compounds isolated after cultivation of a microorganism belonging to the genus Aspergillus, such as lovastatin), U.S. Pat. No. 4,444,784 (synthetic derivatives of the aforementioned compounds such as simvastatin), U.S. Pat. No. 4,739,073 (substituted indoles such as fluvastatin), U.S. Pat. No. 4,346,227 (ML-236B derivatives such as pravastatin), European patent application publication No. 491 226 A (pyridyldihydroxyheptenoic acids such as cerivastatin), U.S. Pat. No. 5,273,995 (6-[2-(substituted-pyrrol-1-yl)alkyl]pyran-2-ones such as atorvastatin and pharmaceutically acceptable forms thereof (i.e. Lipitor®)) Additional HMG-CoA reductase inhibitors of interest herein include rosuvastatin and pitavastatin. All of the references cited above are incorporated herein by reference.

Preferred HMG-CoA reductase inhibitors include lovastatin, simvastatin, pravastatin, fluvastatin, atorvastatin or rivastatin; more preferably, atorvastatin, particularly atorvastatin hemicalcium.

Any compound having activity as a CETP inhibitor can serve as the second compound in the combination therapy aspect of the present invention. The term CETP inhibitor refers to compounds that inhibit the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). CETP inhibitors useful in the combination aspect of the present invention include those disclosed in U.S. Pat. Nos. 6,140,343 and 6,197,786. CETP inhibitors disclosed in these patents include compounds such as [2R,4S]-4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, which is also known as torcetrapib. Also of interest are the CETP inhibitors disclosed in U.S. patent application Ser. No. 60/458,274, filed Mar. 28, 2003, U.S. Pat. No. 5,512,548 (polypeptide derivatives), J. Antibiot., 49(8): 815-816 (1996) (rosenonolactone derivatives) and Bioorg. Med. Chem. Lett.; 6:1951-1954 (1996) (phosphate-containing analogs of cholesteryl ester). All of the references cited above are incorporated herein by reference.

Any PPAR modulator may be used as the second compound in the combination aspect of this invention. The term PPAR modulator refers to compounds which modulate peroxisome proliferator activator receptor (PPAR) activity in mammals, particularly humans. Such modulation may be readily determined by standard assays known in the art. It is believed that such compounds, by modulating the PPAR receptor, stimulate transcription of key genes involved in fatty acid oxidation and genes involved in high density lipoprotein (HDL) assembly (for example, apolipoprotein Al gene transcription), accordingly reducing whole body fat and increasing HDL cholesterol. By virtue of their activity, these compounds also reduce plasma levels of triglycerides, VLDL cholesterol, LDL cholesterol and their associated components and increase HDL cholesterol and apolipoprotein Al. Hence, these compounds are useful for the treatment and correction of the various dyslipidemias associated with the development and incidence of atherosclerosis and cardiovascular disease, including hypoalphalipoproteinemia and hypertriglyceridemia. PPAR.alpha. activators of interest herein include those disclosed in PCT patent application publication Nos. WO 02/064549 and WO 02/064130 and U.S. patent application Ser. No. 10/720,942, filed Nov. 24, 2003. All of the references cited above are incorporated herein by reference.

Any HMG-CoA synthase inhibitor may be used as the second compound in the combination aspect of this invention. The term HMG-CoA synthase inhibitor refers to compounds that inhibit the biosynthesis of hydroxymethylglutaryl-coenzyme A from acetyl-coenzyme A and acetoacetyl-coenzyme A, catalyzed by the enzyme HMG-CoA synthase. Such inhibition is readily determined by standard assays known in the art. (Meth Enzymol. 1975; 35:155-160: Meth. Enzymol. 1985; 110:19-26 and references cited therein). HMG-CoA synthase inhibitors of interest include those disclosed in U.S. Pat. No. 5,120,729 (beta-lactam derivatives), U.S. Pat. No. 5,064,856 (spiro-lactone derivatives prepared by culturing a microorganism (MF5253)) and U.S. Pat. No. 4,847,271 (certain oxetane compounds such as 11-(3-hydroxymethyl-4-oxo-2-oxetayl)-3,5,7-trimethyl-2,4-undeca-dienoic acid derivatives). All of the references cited above are incorporated herein by reference.

Any compound that decreases HMG-CoA reductase gene expression may be used as the second compound in the combination aspect of this invention. These agents may be HMG-CoA reductase transcription inhibitors that block the transcription of DNA or translation inhibitors that prevent or decrease translation of mRNA coding for HMG-CoA reductase into protein. Such compounds may either affect transcription or translation directly, or may be biotransformed to compounds that have the aforementioned activities by one or more enzymes in the cholesterol biosynthetic cascade or may lead to the accumulation of an isoprene metabolite that has the aforementioned activities. Such regulation is readily determined by those skilled in the art according to standard assays (Meth. Enzymol., 1985; 110:9-19). U.S. Pat. No. 5,041,432 discloses certain 15-substituted lanosterol derivatives that decrease HMG-CoA reductase gene expression. Other oxygenated sterols that suppress synthesis of HMG-CoA reductase are discussed by E.I. Mercer (Prog. Lip. Res. 1993; 32:357-416). The references cited above are incorporated herein by reference.

Squalene synthetase inhibitors are also useful in the practice of the combination aspect of the invention. Such compounds inhibit the condensation of 2 molecules of farnesylpyrophosphate to form squalene, catalyzed by the enzyme squalene synthetase. Standard assays for determining squalene synthetase inhibition are well known in the art. (Meth. Enzymol. 1969; 15: 393-454 and Meth. Enzymol. 1985; 110:359-373 and references contained therein. Squalene synthetase inhibitors of interest herein include those disclosed in U.S. Pat. No. 5,026,554 (fermentation products of the microorganism MF5465 (ATCC 74011) including zaragozic acid) as well as those included in the summary of patented squalene synthetase inhibitors which appears in Curr. Op. Ther. Patents (1993) 861-4. The references cited above are incorporated herein by reference.

Any squalene epoxidase inhibitor may be used as the second compound in the combination aspect of this invention. These compounds inhibit the bioconversion of squalene and molecular oxygen into squalene-2,3-epoxide, catalyzed by the enzyme squalene epoxidase. Such inhibition is readily determined by those skilled in the art according to standard assays (Biochim. Biophys. Acta 1984; 794:466-471). squalene epoxidase inhibitors of interest herein include those disclosed in U.S. Pat. Nos. 5,011,859 and 5,064,864 (fluoro analogs of squalene), European patent application publication No. 395,768 A (substituted allylamine derivatives), PCT patent application publication No. WO 93/12069 A (amino alcohol derivatives) and U.S. Pat. No. 5,051,534 (cyclopropyloxy-squalene derivatives). All of the references cited above are incorporated herein by reference.

Squalene cyclase inhibitors are also contemplated herein as a viable pharmaceutical agent for use in the combination aspect of the invention. These compounds inhibit the bioconversion of squalene-2,3-epoxide to lanosterol, catalyzed by the enzyme squalene cyclase. Such inhibition is readily determined by standard assays well known in the art. (FEBS Lett. 1989; 244:347-350.). Squalene cyclase inhibitors of interest include those disclosed in PCT patent application publication No. WO 94/10150 (1,2,3,5,6,7,8,8a-octahydro-5,5,8(beta)-trimethyl-6-isoquinolineamine derivatives, such as N-trifluoroacetyl-1,2,3,5,6,7,8,8a-octahydro-2-allyl-5,5,8(beta)-trimethyl-1-6(beta)-isoquinolineamine) and French patent application publication No. 2697250 (beta, beta-dimethyl-4-piperidine ethanol derivatives such as 1-(1,5,9-trimethyldecyl)-beta, beta-dimethyl-4-piperidineethanol). The references cited above are incorporated herein by reference.

Any combined squalene epoxidase/squalene cyclase inhibitor may be used as the second component in the combination aspect of this invention. The term combined squalene epoxidase/squalene cyclase inhibitor refers to compounds that inhibit the bioconversion of squalene to lanosterol via a squalene-2,3-epoxide intermediate. Combined squalene epoxidase/squalene cyclase inhibiton is readily determined in standard assays for squalene cyclase inhibitors or squalene epoxidase inhibitors. Squalene epoxidase/squalene cyclase inhibitors useful in the practice of the combination aspect of the invention include those disclosed in U.S. Pat. Nos. 5,084,461 and 5,278,171 (azadecalin derivatives), European patent application publication No. 468,434 (piperidyl ether and thio-ether derivatives such as 2-(1-piperidyl)pentyl isopentyl sulfoxide and 2-(1-piperidyl)ethyl ethyl sulfide), PCT patent application publication No. WO 94/01404 (acyl-piperidines such as 1-(1-oxopentyl-5-phenylthio)-4-(2-hydroxy-1-methyl)-ethyl)piperidine) and U.S. Pat. No. 5,102,915 (cyclopropyloxy-squalene derivatives). All of the references cited above are incorporated herein by reference.

The compounds of the present invention can also be administered in combination with naturally occurring substances that act to lower plasma cholesterol levels. These naturally occurring materials are commonly called nutraceuticals and include, for example, garlic extract, Hoodia plant extracts and niacin.

Cholesterol absorption inhibitors may also be used in the combination aspect of the present invention. The term cholesterol absorption inhibition refers to the ability of a compound to prevent cholesterol contained within the lumen of the intestine from entering into the intestinal cells and/or passing from within the intestinal cells into the blood stream. Such cholesterol absorption inhibition activity is readily determined in standard assays (e.g., J. Lipid Res. (1993) 34: 377-395). Cholesterol absorption inhibitors of interest include those disclosed in PCT patent application publication No. WO 94/00480. A preferred cholesterol absorption inhibitor is Zetia™ (ezetimibe) (Merck/Schering-Plough). The references cited above are incorporated herein by reference.

Any ACAT inhibitor may serve as the second compound in the combination therapy aspect of the present invention. The term ACAT inhibitor refers to compounds that inhibit the intracellular esterification of dietary cholesterol by the enzyme acyl CoA: cholesterol acyltransferase. Such inhibition may be determined by standard assays, such as the method of Heider et al. described in Journal of Lipid Research., 24:1127 (1983). ACAT inhibitors useful herein include those disclosed in U.S. Pat. No. 5,510,379 (carboxysulfonates) and PCT patent application publication Nos. WO 96/26948 and WO 96/10559 (both disclose urea derivatives). Preferred ACAT inhibitors include avasimibe (Pfizer), CS-505 (Sankyo) and eflucimibe (Eli Lilly and Pierre Fabre). All of the references cited above are incorporated herein by reference.

Other compounds that are marketed for hyperlipidemia, including hypercholesterolemia, and which are intended to help prevent or treat atherosclerosis and are of interest herein include bile acid sequestrants, such as Welchol®, Colestid®, LoCholest® and Questran®; and fibric acid derivatives, such as Atromid®, Lopid® and Tricor®.

Diabetes (especially Type II), insulin resistance, impaired glucose tolerance, or the like, and any of the diabetic complications such as neuropathy, nephropathy, retinopathy or cataracts may be treated by the administration of a therapeutically effective amount of a compound of Formula I in combination with one or more other agents (e.g., insulin) that are useful in treasting diabetes.

Any glycogen phosphorylase inhibitor may be used as the second agent in combination with a Formula I compound of the present invention. The term glycogen phosphorylase inhibitor refers to compounds that inhibit the bioconversion of glycogen to glucose-1-phosphate, which is catalyzed by the enzyme glycogen phosphorylase. Such glycogen phosphorylase inhibition activity is readily determined by standard assays well known in the art (e.g., J. Med. Chem. 41 (1998) 2934-2938). Glycogen phosphorylase inhibitors of interest herein include those described in PCT patent application publication Nos. WO 96/39384 and WO 96/39385. The references cited above are incorporated herein by reference.

Aldose reductase inhibitors are also useful in the practice of the combination aspect of the present invention. These compounds inhibit the bioconversion of glucose to sorbitol, which is catalyzed by the enzyme aldose reductase. Aldose reductase inhibition is readily determined by standard assays (e.g., J. Malone, Diabetes, 29:861-864 (1980) “Red Cell Sorbitol, an Indicator of Diabetic Control”, incorporated herein by reference). A variety of aldose reductase inhibitors are known to those skilled in the art. The reference cited above are incorporated herein by reference.

Any sorbitol dehydrogenase inhibitor may be used in combination with a Formula I compound of the present invention. The term sorbitol dehydrogenase inhibitor refers to compounds that inhibit the bioconversion of sorbitol to fructose, which is catalyzed by the enzyme sorbitol dehydrogenase. Such sorbitol dehydrogenase inhibitor activity is readily determined by the use of standard assays well known in the art (e.g., Analyt. Biochem (2000) 280: 329-331). Sorbitol dehydrogenase inhibitors of interest include those disclosed in U.S. Pat. Nos. 5,728,704 and 5,866,578. The references cited above are incorporated herein by reference.

Any glucosidase inhibitor can be used in the combination aspect of the present invention. Such compounds inhibit the enzymatic hydrolysis of complex carbohydrates by glycoside hydrolases such as amylase or maltase into bioavailable simple sugars, for example, glucose. The rapid metabolic action of glucosidases, particularly following the intake of high levels of carbohydrates, results in a state of alimentary hyperglycemia, which, in adipose or diabetic subjects, leads to enhanced secretion of insulin, increased fat synthesis and a reduction in fat degradation. Following such hyperglycemias, hypoglycemia frequently occurs, due to the augmented levels of insulin present. Additionally, it is known that chyme remaining in the stomach promotes the production of gastric juice, which initiates or favors the development of gastritis or duodenal ulcers. Accordingly, glucosidase inhibitors are known to have utility in accelerating the passage of carbohydrates through the stomach and inhibiting the absorption of glucose from the intestine. Furthermore, the conversion of carbohydrates into lipids of the fatty tissue and the subsequent incorporation of alimentary fat into fatty tissue deposits is accordingly reduced or delayed, with the concomitant benefit of reducing or preventing the deleterious abnormalities resulting therefrom. Such glucosidase inhibition activity is readily determined by those skilled in the art according to standard assays (e.g., Biochemistry (1969)8: 4214), incorporated herein by reference.

A generally preferred glucosidase inhibitor includes an amylase inhibitor. An amylase inhibitor is a glucosidase inhibitor that inhibits the enzymatic degradation of starch or glycogen into maltose. Such amylase inhibition activity is readily determined by use of standard assays (e.g., Methods Enzymol. (1955)1: 149, incorporated herein by reference). The inhibition of such enzymatic degradation is beneficial in reducing amounts of bioavailable sugars, including glucose and maltose, and the concomitant deleterious conditions resulting therefrom.

Preferred glucosidase inhibitors include acarbose, adiposine, voglibose, miglitol, emiglitate, camiglibose, tendamistate, trestatin, pradimicin-Q and salbostatin. The glucosidase inhibitor acarbose and various amino sugar derivatives related thereto are disclosed in U.S. Pat. Nos. 4,062,950 and 4,174,439 respectively. The glucosidase inhibitor adiposine is disclosed in U.S. Pat. No. 4,254,256. The glucosidase inhibitor voglibose, 3,4-dideoxy-4-[[2-hydroxy-1-(hydroxymethyl)ethyl]amino]-2-C-(hydroxymethyl)-D-epi-inositol, and various N-substituted pseudo-aminosugars related thereto are disclosed in U.S. Pat. No. 4,701,559. The glucosidase inhibitor miglitol, (2R,3R,4R,5S)-1-(2-hydroxyethyl)-2-(hydroxymethyl)-3,4,5-piperidinetriol, and various 3,4,5-trihydroxypiperidines related thereto are disclosed in U.S. Pat. No. 4,639,436. The glucosidase inhibitor emiglitate, ethyl p[2-[(2R,3R,4R,5S)-3,4,5-trihydroxy-2-(hydroxymethyl)piperidino]ethoxy]-benzoate, various derivatives related thereto and pharmaceutically acceptable acid addition salts thereof are disclosed in U.S. Pat. No. 5,192,772. The glucosidase inhibitor MDL-25637,2,6-dideoxy-7-O-.beta.-D-glucopyranosyl-2,6-imino-D-glycero-L-gluco-heptitol, various homodisaccharides related thereto and the pharmaceutically acceptable acid addition salts thereof are disclosed in U.S. Pat. No. 4,634,765. The glucosidase inhibitor camiglibose, methyl 6-deoxy-6-[(2R,3R,4R,5S)-3,4,5-trihydroxy-2-(hydroxymethyl)piperidino]-.alpha.-D-glucopyranoside sesquihydrate, deoxynojirimycin derivatives related thereto, various pharmaceutically acceptable salts thereof and synthetic methods for the preparation thereof are disclosed in U.S. Pat. Nos. 5,157,116 and 5,504,078. The glycosidase inhibitor salbostatin and various pseudosaccharides related thereto are disclosed in U.S. Pat. No. 5,091,524. All of the references cited above are incorporated herein by reference.

Amylase inhibitors of interest herein are disclosed in U.S. Pat. No. 4,451,455, U.S. Pat. No. 4,623,714 (Al-3688 and the various cyclic polypeptides related thereto) and U.S. Pat. No. 4,273,765 (trestatin, which consists of a mixture of trestatin A, trestatin B and trestatin C, and the various trehalose-containing aminosugars related theret). All of the references cited above are incorporated herein by reference.

Additional anti-diabetic compounds, which may be used as the second agent in combination with a Formula I compound of the present invention, include, for example,

the following: biguanides (e.g., metformin, pfenformin or buformin), insulin secretagogues (e.g., sulfonylureas and glinides), glitazones, non-glitazone PPARγ agonists, PPAR.beta. agonists, inhibitors of DPP-IV (i.e., sitagliptin, vilagliptin, saxagliptin, linagliptin, alogliptin, and berberine), inhibitors of PDE5, inhibitors of GSK-3, glucagon antagonists, inhibitors of f-1,6-BPase (Metabasis/Sankyo), GLP-1/analogs (AC 2993, also known as exendin-4), insulin and insulin mimetics (Merck natural products). Other examples would include PKC-.beta. inhibitors and AGE breakers.

The Formula I compounds of the present invention may also be used in combination with antihypertensive agents Preferred antihypertensive agents useful in the present invention include calcium channel blockers, such as Cardizeme®, Adalat®, Calan®, Cardene®, Covera®, Dilacor®, DynaCirce Procardia XL®, Sular®, Tiazac®, Vascor®, Verelan®, Isoptin®, Nimotop®, Norvasc®, and Plendil®; angiotensin converting enzyme (ACE) inhibitors, such as Accupril®, Altace®, Captopril®, Lotensin®, Mavik®, Monopril®, Prinivil®, Univasc®, Vasotec® and Zestril®.

The additional pharmaceutical agent is preferably an anti-obesity agent as described above, but otherwise will frequently be an HMG-CoA reductase inhibitor, an HMG-CoA synthase inhibitor, an inhibitor of HMG-CoA reductase gene expression, a CETP inhibitor, a PPAR modulator, a squalene synthetase inhibitor, a squaline epoxidase inhibitor, a squaline cyclase inhibitor, a combined squaline epoxidase/cyclase inhibitor, a cholesterol absorption inhibitor, an ACAT inhibitor, a pancreatic lipase inhibitor, a gastric lipase inhibitor, a calcium channel blocker, an ACE inhibitor, a beta blocker, a diuretic, niacin, a garlic extract preparation, a bile acid sequestrant, a fibric acid derivative, a glycogen phosphorylase inhibitor, an aldose reductase inhibitor, a sorbitol dehydrogenase inhibitor, a glucosidase inhibitoran amylase inhibitor or a DPP-IV inhibitor (i.e., sitagliptin, vilagliptin, saxagliptin, linagliptin, alogliptin, and berberine).

The dosage of the additional pharmaceutical agent is generally dependent upon a number of factors including the health of the subject being treated, the extent of treatment desired, the nature and kind of concurrent therapy, if any, and the frequency of treatment and the nature of the effect desired. In general, the dosage range of the additional pharmaceutical agent is in the range of from about 0.001 mg to about 100 mg per kilogram body weight of the individual per day, preferably from about 0.1 mg to about 10 mg per kilogram body weight of the individual per day. However, some variability in the general dosage range may also be required depending upon the age and weight of the subject being treated, the intended route of administration, the particular anti-obesity agent being administered and the like. The determination of dosage ranges and optimal dosages for a particular patient is also well within the ability of one of ordinary skill in the art having the benefit of the instant disclosure.

According to the methods of treatment of the invention, a compound of the present invention or a combination of a compound of the present invention and at least one additional pharmaceutical agent (referred to herein as a “combination”) is administered to a subject in need of such treatment, preferably in the form of a pharmaceutical composition. In the combination aspect of the invention, the compound of the present invention and at least one other pharmaceutical agent (e.g., another anti-obesity agent,) may be administered either separately or in a pharmaceutical composition comprising both. It is generally preferred that such administration be oral.

When a combination of a compound of the present invention and at least one other pharmaceutical agent are administered together, such administration may be sequential in time or simultaneous. Simultaneous administration of drug combinations is generally preferred. For sequential administration, a compound of the present invention and the additional pharmaceutical agent may be administered in any order. It is generally preferred that such administration be oral. It is especially preferred that such administration be oral and simultaneous. When a compound of the present invention and the additional pharmaceutical agent are administered sequentially, the administration of each may be by the same or by different methods.

According to the methods of the invention, a compound of the present invention or a combination is preferably administered in the form of a pharmaceutical composition. Accordingly, a compound of the present invention or a combination can be administered to a patient separately or together in any conventional oral, rectal, transdermal, parenteral (e.g., intravenous, intramuscular or subcutaneous), intracisternal, intravaginal, intraperitoneal, topical (e.g., powder, ointment, cream, spray or lotion), buccal or nasal dosage form (e.g., spray, drops or inhalant).

The compounds of the invention or combinations can be administered alone but will generally be administered in an admixture with one or more suitable pharmaceutical excipients, adjuvants, diluents or carriers known in the art and selected with regard to the intended route of administration and standard pharmaceutical practice. The compound of the invention or combination may be formulated to provide immediate-, delayed-, modified-, sustained-, pulsed- or controlled-release dosage forms depending on the desired route of administration and the specificity of release profile, commensurate with therapeutic needs.

The pharmaceutical composition comprises a compound of the invention or a combination in an amount generally in the range of from about 1% to about 75%, 80%, 85%, 90% or even 95% (by weight) of the composition, usually in the range of about 1%, 2% or 3% to about 50%, 60% or 70%, more frequently in the range of about 1%, 2% or 3% to less than 50% such as about 25%, 30% or 35%.

Methods of preparing various pharmaceutical compositions with a specific amount of active compound are known to those skilled in this art. For examples, see Remington: The Practice of Pharmacy, Lippincott Williams and Wilkins, Baltimore Md. 20.sup.th ed. 2000.

Compositions suitable for parenteral injection generally include pharmaceutically acceptable sterile aqueous or nonaqueous solutions, dispersions, suspensions, or emulsions, and sterile powders for reconstitution into sterile injectable solutions or dispersions. Examples of suitable aqueous and nonaqueous carriers or diluents (including solvents and vehicles) include water, ethanol, polyols (propylene glycol, polyethylene glycol, glycerol, and the like), suitable mixtures thereof, triglycerides including vegetable oils such as olive oil, and injectable organic esters such as ethyl oleate. A preferred carrier is Miglyol® brand caprylic/capric acid ester with glycerine or propylene glycol (e.g., Miglyol® 812, Miglyol® 829, Miglyol® 840) available from Condea Vista Co., Cranford, N.J. Proper fluidity can be maintained, for example, by the use of a coating such as lecithin, by the maintenance of the required particle size in the case of dispersions, and by the use of surfactants.

These compositions for parenteral injection may also contain excipients such as preserving, wetting, emulsifying, and dispersing agents. Prevention of microorganism contamination of the compositions can be accomplished with various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and gelatin.

Solid dosage forms for oral administration include capsules, tablets, chews, lozenges, pills, powders, and multi-particulate preparations (granules). In such solid dosage forms, a compound of the present invention or a combination is admixed with at least one inert excipient, diluent or carrier. Suitable excipients, diluents or carriers include materials such as sodium citrate or dicalcium phosphate and/or (a) one or more fillers or extenders (e.g., microcrystalline cellulose (available as Avicel™ from FMC Corp.) starches, lactose, sucrose, mannitol, silicic acid, xylitol, sorbitol, dextrose, calcium hydrogen phosphate, dextrin, alpha-cyclodextrin, beta-cyclodextrin, polyethylene glycol, medium chain fatty acids, titanium oxide, magnesium oxide, aluminum oxide and the like); (b) one or more binders (e.g., carboxymethylcellulose, methylcellulose, hydroxypropylcellulose, hydroxypropylmethylcellulose, gelatin, gum arabic, ethyl cellulose, polyvinyl alcohol, pullulan, pregelatinized starch, agar, tragacanth, alginates, gelatin, polyvinylpyrrolidone, sucrose, acacia and the like); (c) one or more humectants (e.g., glycerol and the like); (d) one or more disintegrating agents (e.g., agar-agar, calcium carbonate, potato or tapioca starch, alginic acid, certain complex silicates, sodium carbonate, sodium lauryl sulphate, sodium starch glycolate (available as Explotab™ from Edward Mendell Co.), cross-linked polyvinyl pyrrolidone, croscarmellose sodium A-type (available as Ac-di-sol™), polyacrilin potassium (an ion exchange resin) and the like); (e) one or more solution retarders (e.g., paraffin and the like); (f) one or more absorption accelerators (e.g., quaternary ammonium compounds and the like); (g) one or more wetting agents (e.g., cetyl alcohol, glycerol monostearate and the like); (h) one or more adsorbents (e.g., kaolin, bentonite and the like); and/or (i) one or more lubricants (e.g., talc, calcium stearate, magnesium stearate, stearic acid, polyoxyl stearate, cetanol, talc, hydrogenated caster oil, sucrose esters of fatty acid, dimethylpolysiloxane, microcrystalline wax, yellow beeswax, white beeswax, solid polyethylene glycols, sodium lauryl sulfate and the like). In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules may be prepared with coatings and shells, such as enteric coatings and others well known in the art. They may also contain opacifying agents, and can also be of such composition that they release the compound of the present invention and/or the additional pharmaceutical agent in a delayed manner. Examples of embedding compositions that can be used are polymeric substances and waxes. The drug may also be in micro-encapsulated form, if appropriate, with one or more of the above-mentioned excipients.

For tablets, the active agent will typically comprise less than 50% (by weight) of the formulation, for example less than about 10% such as 5% or 2.5% by weight. The predominant portion of the formulation comprises fillers, diluents, disintegrants, lubricants and optionally, flavors. The composition of these excipients is well known in the art. Frequently, the fillers/diluents will comprise mixtures of two or more of the following components: microcrystalline cellulose, mannitol, lactose (all types), starch, and di-calcium phosphate. The filler/diluent mixtures typically comprise less than 98% of the formulation and preferably less than 95%, for example 93.5%. Preferred disintegrants include Ac-di-sol™, Explotab™, starch and sodium lauryl sulphate. When present a disintegrant will usually comprise less than 10% of the formulation or less than 5%, for example about 3%. A preferred lubricant is magnesium stearate. When present a lubricant will usually comprise less than 5% of the formulation or less than 3%, for example about 1%.

Tablets may be manufactured by standard tabletting processes, for example, direct compression or a wet, dry or melt granulation, melt congealing process and extrusion. The tablet cores may be mono or multi-layer(s) and can be coated with appropriate overcoats known in the art.

Liquid dosage forms for oral administration include pharmaceutically acceptable emulsions, solutions, suspensions, syrups, and elixirs. In addition to the compound of the present invention or the combination, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed oil, groundnut oil, corn germ oil, olive oil, castor oil, sesame seed oil and the like), Miglyole® (available from CONDEA Vista Co., Cranford, N.J.), glycerol, tetrahydrofurfuryl alcohol, polyethylene glycols and fatty acid esters of sorbitan, or mixtures of these substances, and the like.

Besides such inert diluents, the composition may also include excipients, such as wetting agents, emulsifying and suspending agents, sweetening, flavoring, and perfuming agents.

Oral liquid forms of the compounds of the invention or combinations include solutions, wherein the active compound is fully dissolved. Examples of solvents include all pharmaceutically precedented solvents suitable for oral administration, particularly those in which the compounds of the invention show good solubility, e.g., polyethylene glycol, polypropylene glycol, edible oils and glyceryl- and glyceride-based systems. Glyceryl- and glyceride-based systems may include, for example, the following branded products (and corresponding generic products): Captex™ 355 EP (glyceryl tricaprylate/caprate, from Abitec, Columbus Ohio), Crodamol™ GTC/C (medium chain triglyceride, from Croda, Cowick Hall, UK) or Labrafac™ CC (medium chain triglyides, from Gattefosse), Captex™ 500P (glyceryl triacetate i.e. triacetin, from Abitec), Capmul™ MCM (medium chain mono- and diglycerides, from Abitec), Migyol™ 812 (caprylic/capric triglyceride, from Condea, Cranford N.J.), Migyol™ 829 (caprylic/capric/succinic triglyceride, from Condea), Migyol™ 840 (propylene glycol dicaprylate/dicaprate, from Condea), Labrafil™ M1944CS (oleoyl macrogol-6 glycerides, from Gattefosse), Peceol™ (glyceryl monooleate, from Gattefosse) and Maisine™ 35-1 (glyceryl monooleate, from Gattefosse). Of particular interest are the medium chain (about C.sub.8 to C.sub.10) triglyceride oils. These solvents frequently make up the predominant portion of the composition, i.e., greater than about 50%, usually greater than about 80%, for example about 95% or 99%. Adjuvants and additives may also be included with the solvents principally as taste-mask agents, palatability and flavoring agents, antioxidants, stabilizers, texture and viscosity modifiers and solubilizers.

Suspensions, in addition to the compound of the present invention or the combination, may further comprise carriers such as suspending agents, e.g., ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal or vaginal administration preferably comprise suppositories, which can be prepared by mixing a compound of the present invention or a combination with suitable non-irritating excipients or carriers, such as cocoa butter, polyethylene glycol or a suppository wax which are solid at ordinary room temperature, but liquid at body temperature, and therefore, melt in the rectum or vaginal cavity thereby releasing the active component(s).

Dosage forms for topical administration of the compounds of the present invention or combinations include ointments, creams, lotions, powders and sprays. The drugs are admixed with a pharmaceutically acceptable excipient, diluent or carrier, and any preservatives, buffers, or propellants that may be required.

Many of the present compounds are poorly soluble in water, e.g., less than about 1 .mu.g/mL. Therefore, liquid compositions in solubilizing, non-aqueous solvents such as the medium chain triglyceride oils discussed above are a preferred dosage form for these compounds.

Solid amorphous dispersions, including dispersions formed by a spray-drying process, are also a preferred dosage form for the poorly soluble compounds of the invention. By “solid amorphous dispersion” is meant a solid material in which at least a portion of the poorly soluble compound is in the amorphous form and dispersed in a water-soluble polymer. By “amorphous” is meant that the poorly soluble compound is not crystalline. By “crystalline” is meant that the compound exhibits long-range order in three dimensions of at least 100 repeat units in each dimension. Thus, the term amorphous is intended to include not only material which has essentially no order, but also material which may have some small degree of order, but the order is in less than three dimensions and/or is only over short distances. Amorphous material may be characterized by techniques known in the art such as powder x-ray diffraction (PXRD) crystallography, solid state NMR, or thermal techniques such as differential scanning calorimetry (DSC).

Preferably, at least a major portion (i.e., at least about 60 wt %) of the poorly soluble compound in the solid amorphous dispersion is amorphous. The compound can exist within the solid amorphous dispersion in relatively pure amorphous domains or regions, as a solid solution of the compound homogeneously distributed throughout the polymer or any combination of these states or those states that lie intermediate between them. Preferably, the solid amorphous dispersion is substantially homogeneous so that the amorphous compound is dispersed as homogeneously as possible throughout the polymer. As used herein, “substantially homogeneous” means that the fraction of the compound that is present in relatively pure amorphous domains or regions within the solid amorphous dispersion is relatively small, on the order of less than 20 wt %, and preferably less than 10 wt % of the total amount of drug.

Water-soluble polymers suitable for use in the solid amorphous dispersions should be inert, in the sense that they do not chemically react with the poorly soluble compound in an adverse manner, are pharmaceutically acceptable, and have at least some solubility in aqueous solution at physiologically relevant pHs (e.g. 1-8). The polymer can be neutral or ionizable, and should have an aqueous-solubility of at least 0.1 mg/mL over at least a portion of the pH range of 1-8.

Water-soluble polymers suitable for use with the present invention may be cellulosic or non-cellulosic. The polymers may be neutral or ionizable in aqueous solution. Of these, ionizable and cellulosic polymers are preferred, with ionizable cellulosic polymers being more preferred.

Exemplary water-soluble polymers include hydroxypropyl methyl cellulose acetate succinate (HPMCAS), hydroxypropyl methyl cellulose (HPMC), hydroxypropyl methyl cellulose phthalate (HPMCP), carboxy methyl ethyl cellulose (CMEC), cellulose acetate phthalate (CAP), cellulose acetate trimellitate (CAT), polyvinylpyrrolidone (PVP), hydroxypropyl cellulose (HPC), methyl cellulose (MC), block copolymers of ethylene oxide and propylene oxide (PEO/PPO, also known as poloxamers), and mixtures thereof. Especially preferred polymers include HPMCAS, HPMC, HPMCP, CMEC, CAP, CAT, PVP, poloxamers, and mixtures thereof. Most preferred is HPMCAS. See European Patent Application Publication No. 0 901 786 A2, the disclosure of which is incorporated herein by reference.

The solid amorphous dispersions may be prepared according to any process for forming solid amorphous dispersions that results in at least a major portion (at least 60%) of the poorly soluble compound being in the amorphous state. Such processes include mechanical, thermal and solvent processes. Exemplary mechanical processes include milling and extrusion; melt processes including high temperature fusion, solvent-modified fusion and melt-congeal processes; and solvent processes including non-solvent precipitation, spray coating and spray drying. See, for example, the following U.S. patents, the pertinent disclosures of which are incorporated herein by reference: U.S. Pat. Nos. 5,456,923 and 5,939,099, which describe forming dispersions by extrusion processes; U.S. Pat. Nos. 5,340,591 and 4,673,564, which describe forming dispersions by milling processes; and U.S. Pat. Nos. 5,707,646 and 4,894,235, which describe forming dispersions by melt congeal processes. In a preferred process, the solid amorphous dispersion is formed by spray drying, as disclosed in European Patent Application Publication No. 0 901 786 A2. In this process, the compound and polymer are dissolved in a solvent, such as acetone or methanol, and the solvent is then rapidly removed from the solution by spray drying to form the solid amorphous dispersion. The solid amorphous dispersions may be prepared to contain up to about 99 wt % of the compound, e.g., 1 wt %, 5 wt %, 10 wt %, 25 wt %, 50 wt %, 75 wt %, 95 wt %, or 98 wt % as desired.

The solid dispersion may be used as the dosage form itself or it may serve as a manufacturing-use-product (MUP) in the preparation of other dosage forms such as capsules, tablets, solutions or suspensions. An example of an aqueous suspension is an aqueous suspension of a 1:1 (w/w) compound/HPMCAS-HF spray-dried dispersion containing 2.5 mg/mL of compound in 2% polysorbate-80. Solid dispersions for use in a tablet or capsule will generally be mixed with other excipients or adjuvants typically found in such dosage forms. For example, an exemplary filler for capsules contains a 2:1 (w/w) compound/HPMCAS-MF spray-dried dispersion (60%), lactose (fast flow) (15%), microcrystalline cellulose (e.g., Avicel.sup.(R0-102) (15.8%), sodium starch (7%), sodium lauryl sulfate (2%) and magnesium stearate (1%).

The HPMCAS polymers are available in low, medium and high grades as Aqoa.sup.(R)-LF, Aqoat.sup.(R)-MF and Aqoat.sup.(R)-HF respectively from Shin-Etsu Chemical Co., LTD, Tokyo, Japan. The higher MF and HF grades are generally preferred.

The following paragraphs describe exemplary formulations, dosages, etc. useful for non-human animals. The administration of the compounds of the present invention and combinations of the compounds of the present invention with anti-obesity agents can be effected orally or non-orally.

An amount of a compound of the present invention or combination of a compound of the present invention with another anti-obesity agent is administered such that an effective dose is received. Generally, a daily dose that is administered orally to an animal is between about 0.01 and about 1,000 mg/kg of body weight, e.g., between about 0.01 and about 300 mg/kg or between about 0.01 and about 100 mg/kg or between about 0.01 and about 50 mg/kg of body weight, or between about 0.01 and about 25 mg/kg, or about 0.01 and about 10 mg/kg or about 0.01 and about 5 mg/kg.

Conveniently, a compound of the present invention (or combination) can be carried in the drinking water so that a therapeutic dosage of the compound is ingested with the daily water supply. The compound can be directly metered into drinking water, preferably in the form of a liquid, water-soluble concentrate (such as an aqueous solution of a water-soluble salt).

Conveniently, a compound of the present invention (or combination) can also be added directly to the feed, as such, or in the form of an animal feed supplement, also referred to as a premix or concentrate. A premix or concentrate of the compound in an excipient, diluent or carrier is more commonly employed for the inclusion of the agent in the feed. Suitable excipients, diluents or carriers are liquid or solid, as desired, such as water, various meals such as alfalfa meal, soybean meal, cottonseed oil meal, linseed oil meal, corncob meal and corn meal, molasses, urea, bone meal, and mineral mixes such as are commonly employed in poultry feeds. A particularly effective excipient, diluent or carrier is the respective animal feed itself; that is, a small portion of such feed. The carrier facilitates uniform distribution of the compound in the finished feed with which the premix is blended. Preferably, the compound is thoroughly blended into the premix and, subsequently, the feed. In this respect, the compound may be dispersed or dissolved in a suitable oily vehicle such as soybean oil, corn oil, cottonseed oil, and the like, or in a volatile organic solvent and then blended with the carrier. It will be appreciated that the proportions of compound in the concentrate are capable of wide variation since the amount of the compound in the finished feed may be adjusted by blending the appropriate proportion of premix with the feed to obtain a desired level of compound.

High potency concentrates may be blended by the feed manufacturer with proteinaceous carrier such as soybean oil meal and other meals, as described above, to produce concentrated supplements, which are suitable for direct feeding to animals. In such instances, the animals are permitted to consume the usual diet. Alternatively, such concentrated supplements may be added directly to the feed to produce a nutritionally balanced, finished feed containing a therapeutically effective level of a compound of the present invention. The mixtures are thoroughly blended by standard procedures, such as in a twin shell blender, to ensure homogeneity.

If the supplement is used as a top dressing for the feed, it likewise helps to ensure uniformity of distribution of the compound across the top of the dressed feed.

Drinking water and feed effective for increasing lean meat deposition and for improving lean meat to fat ratio are generally prepared by mixing a compound of the present invention with a sufficient amount of animal feed to provide from about 10.sub.-3 to about 500 ppm of the compound in the feed or water.

The preferred medicated swine, cattle, sheep and goat feed generally contain from about 1 to about 400 grams of a compound of the present invention (or combination) per ton of feed, the optimum amount for these animals usually being about 50 to about 300 grams per ton of feed.

The preferred poultry and domestic pet feeds usually contain about 1 to about 400 grams and preferably about 10 to about 400 grams of a compound of the present invention (or combination) per ton of feed.

For parenteral administration in animals, the compounds of the present invention (or combination) may be prepared in the form of a paste or a pellet and administered as an implant, usually under the skin of the head or ear of the animal in which increase in lean meat deposition and improvement in lean meat to fat ratio is sought.

Paste Formulations may be prepared by dispersing the drug in a pharmaceutically acceptable oil such as peanut oil, sesame oil, corn oil or the like.

Pellets containing an effective amount of a compound of the present invention, pharmaceutical composition, or combination may be prepared by admixing a compound of the present invention or combination with a diluent such as carbowax, carnuba wax, and the like, and a lubricant, such as magnesium or calcium stearate, may be added to improve the pelleting process.

It is, of course, recognized that more than one pellet may be administered to an animal to achieve the desired dose level which will provide the increase in lean meat deposition and improvement in lean meat to fat ratio desired. Moreover, implants may also be made periodically during the animal treatment period in order to maintain the proper drug level in the animal's body.

The present invention has several advantageous veterinary features. For the pet owner or veterinarian who wishes to increase leanness and/or trim unwanted fat from pet animals, the instant invention provides the means by which this may be accomplished. For poultry, beef and swine breeders, utilization of the method of the present invention yields leaner animals that command higher sale prices from the meat industry.

### Synthesis

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

Compounds of the invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database).

In general, the compounds of this invention can be made by processes which include processes analogous to those known in the chemical arts, particularly in light of the description contained herein. Certain processes for the manufacture of the compounds of this invention are provided as further features of the invention and are illustrated by the following reaction schemes. Other processes are described in the experimental section.

The Reaction Schemes herein described are intended to provide a general description of the methodology employed in the preparation of many of the Examples given. However, it will be evident from the detailed descriptions given in the Experimental section that the modes of preparation employed extend further than the general procedures described herein. In particular, it is noted that the compounds prepared according to these Schemes may be modified further to provide new Examples within the scope of this invention. For example, an ester functionality may be reacted further using procedures well known to those skilled in the art to give another ester, a carboxylic acid, an amide, a carbinol or a ketone.

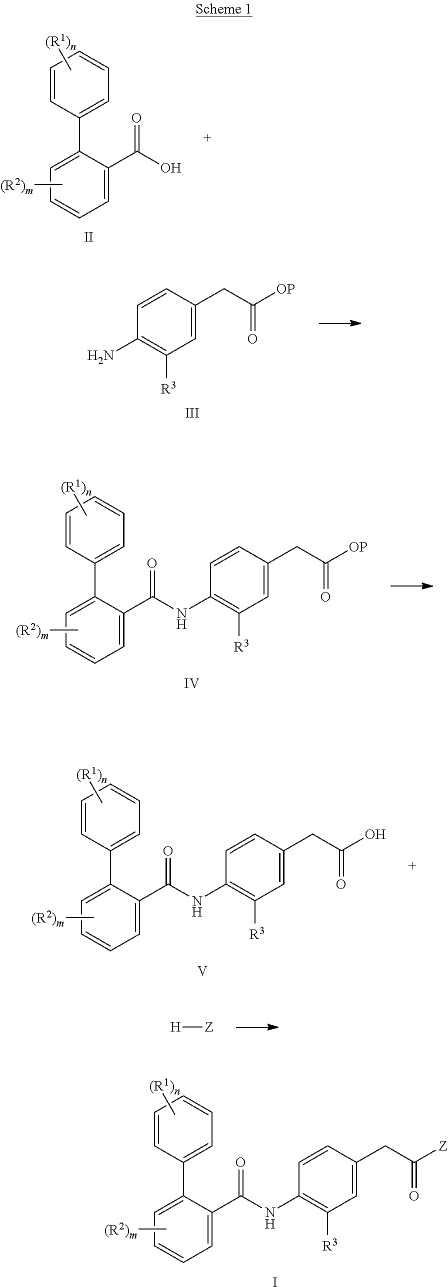

According to reaction Scheme 1, the desired compounds wherein R¹, R², R³, Z, m and n are as described in the Summary may be prepared by initial amide coupling of suitably protected forms of compound II and compound III, wherein the ester P-group is selected from but not limited to a range of suitable groups including methyl, ethyl, isopropyl, tert-butyl, allyl, and benzyl (preferably methyl). Acids of compound II may be purchased, are known in the literature or can be prepared using a variety of methods known to those skilled in the art, including a biaryl coupling reaction involving an aryl halide such as a chloride, bromide or iodide (preferably an iodide) with an aryl metal species (preferably a boronate) catalyzed by a transition metal (preferably palladium). Such coupling reactions are often performed with a suitable carboxylic acid protecting group such as a methyl, ethyl, isopropyl or tert-butyl ester, which is then deprotected, after the biaryl bond is formed, by treatment with hydroxide or aqueous acid (preferably lithium hydroxide in a mixture of methanol, water and THF) in the case of the lower alkyl esters or acid conditions such as trifluoroacetic acid or hydrochloric acid in dioxane for acid labile esters such as tert-butyl to afford acids of formula II. The reaction of acids of formula II with amines of formula III to make amides of formula IV may employ a range of amide coupling conditions known to those skilled in the art, including preparation of the corresponding acid chloride or acyl imidazole, or directly from the acid employing, 1-ethyl-3-(3-dimethylaminopropyl) carbodiimide (EDCI), carbonyldiimidazole (CDI), 2-(1H-7-Azabenzotriazol-1-yl)-1,1,3,3-tetramethyl uronium hexafluorophosphate methanaminium (HATU) or other coupling reagents known to those skilled in the art, preferably preparing the acid chloride by treatment of the carboxylic acid II with oxalyl chloride in a halogenated solvent such as dichloromethane or dichloroethane (preferably dichloroethane) optionally with a catalyst such as pyridine, 4-dimethylaminopyridine, imidazole or an amide (preferentially dimethylformamide) at a range of temperatures between −78° C. and solvent reflux (preferably at 0° C.) and followed by warming to ambient temperature for a period between 5 minutes and 24 hours (preferably 2 hours) followed by removal of the volatiles by concentration under vacuum with the aid of a rotary evaporator. The resulting acid chloride is then dissolved in a halogenated solvent such as dichloromethane or dichloroethane (preferentially dichloroethane) and combined with amine of formula III in a solvent such as dichloromethane or dichloroethane (preferentially dichloroethane) in the presence of a base such as a hydroxide, a carbonate, a pyridine, an amidine, or a teriary amine (preferentially triethylamine). Preferentially the acid chloride of acid II is added as a dichloroethane solution to a stirred solution of the amine III in combination with the base (preferentially triethylamine) at a temperature between −78° C. and solvent reflux (preferentially 0° C.) and stirred at ambient temperature for 1 minute to 24 hours, preferentially 1 hour before working up in the usual manner to provide amides of formula IV.

Acids of formula V may be prepared by treating esters of formula IV under a range of ester cleaving methods known to those skilled in the art including acid or base treatment. For suitably substituted esters (for example when P is equal to benzyl or allyl) hydrogenolytic methods such as treating a solution of the benzyl or allyl ester and a catalyst such as palladium hydroxide or palladium on carbon (preferably 10% palladium on carbon) in an alcoholic solvent (preferably ethanol) with a hydrogen source such as hydrogen gas, ammonium formate or cyclohexene or cyclohexadiene (preferably hydrogen gas) at a temperature between 0° C. and solvent reflux (preferably ambient temperature) and a pressure between 1 and 10 atmospheres (preferably 3 atmospheres). For suitably substituted esters that are cleavable under acid catalysis such as tert-butyl, alkoxybenzyl or diphenylmethano esters (preferably P equal to tert-butyl) acid catalyzed deprotection may be used such as hydrochloric or trifluoroacetic acid (preferably trifluoroacetic acid) optionally in a co-solvent such as dioxane or dichloromethane (preferably dichloromethane) at a temperature between 0 C and solvent reflux (preferably at room temperature) for 5 minutes to 24 hours (preferably 2 hours). For compounds of formula IV where P is a lower alkyl group such as methyl, ethyl, isopropyl (preferably methyl) the ester may be cleaved by treatment with an alkali metal hydroxide, or other methods known to those skilled in the art for cleaving esters to acids (preferably treatment of the ester as a solution in tetrahydrofuran and methanol with an aqueous lithium hydroxide solution, preferably 1 molar) at a temperature between 0 C and solvent reflux (preferably room temperature) for between 5 min and 24 hours (preferably 1 hour) followed by acidic workup to afford the carboxylic acid V. Carboxylic acid V may also be prepared in an analogous fashion from the corresponding malonoyl diester, which after coupling and cleavage will yield the corresponding mono acid V after spontaneous decarboxylation during an acidic workup of the diacid.

Compounds of formula I are obtained from acids of formula V by reacting with the corresponding alcohols of formula VI under dehydrative esterification conditions known to those skilled in the art such as treatment with a carbodiimide (preferably 1-ethyl-3-(3-dimethylaminopropyl)carbodiimide hydrogen chloride salt) and a catalyst (preferably 4-(dimethylamino)-pyridine) in a solvent such as dichloromethane or (preferably 2-methyltetrahydrofuran) at a temperature between 0 C and solvent reflux (preferably ambient temperature) for a period between 1 hour and 48 hours (preferably 5 hours).

Scheme 2 depicts an alternative synthetic route to make compounds of formula I.

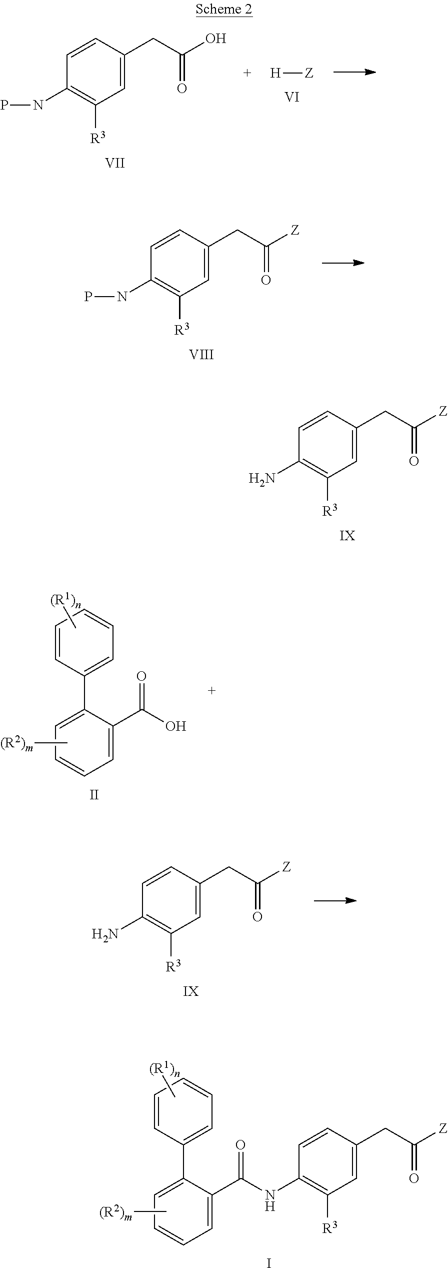

According to the reaction sequence depicted in Scheme 2, the desired compounds may be prepared wherein R¹, R², R³, Z, m and n are as described in the Summary and the P-group is a suitable protecting group including but are not limited to tert-butyl carbamate, benzylcarbamate or an oxidized form of nitrogen such as but not limited to a nitro group. Compounds of formula VIII are obtained from acids of formula VII and alcohols of formula VI under dehydrative esterification conditions known to those skilled in the art such as treatment with a carbodiimide (preferably 1-ethyl-3-(3-dimethylaminopropyl)carbodiimide hydrogen chloride salt) and a catalyst (preferably 4-(dimethylamino)-pyridine) in a solvent such as dichloromethane or (preferably 2-methyltetrahydrofuran) at a temperature between 0° C. and solvent reflux (preferably ambient temperature) for a period between 1 hour and 48 hours (preferably 5 hours).

The nitrogen P-groups in formula VIII can be converted to the amino group in formula IX by a variety of methods known to those skilled in the art including reaction of the tert-butyl carbamate under acidic conditions such as hydrochloric acid in dioxane or a carboxylic acid (preferably trifluoroacetic acid) optionally in the presence of a halogenated solvent (preferably dichloromethane) at a temperature between 0° C. and solvent reflux (preferably room temperature) for 5 minutes to 24 hours (preferably 2 hours). Benzyl or allylic carbamates may be converted to amines of formula IX by employing hydrogenolytic methods such as treating a solution of the benzyl or allyl carbamate and a catalyst such as palladium hydroxide or palladium on carbon (preferably 10% palladium on carbon) in an alcoholic solvent (preferably ethanol) with a hydrogen source such as hydrogen gas, ammonium formate or cyclohexene or cyclohexadiene (preferably hydrogen gas) at a temperature between 0° C. and solvent reflux (preferably ambient temperature) and a pressure between 1 and 10 atmospheres (preferably 3 atmospheres). Oxidized nitrogen P-groups such as nitro of formula VIII may be converted to amines of formula IX by reduction employing a variety of methods known to those skilled in the art such as treating a solution of the nitro group and a catalyst such as palladium on carbon (preferably 10% palladium on carbon) in an alcoholic solvent (preferably ethanol) with a hydrogen source (preferably hydrogen gas) at a temperature between 0° C. and solvent reflux (preferably ambient temperature) and a pressure between 1 and 10 atmospheres (preferably 3 atmospheres). Oxidized nitrogen P-groups such as nitro of formula VIII may also be converted to amines of formula IX by reduction with a reducing metal (preferably iron filings) in a solvent (preferably ethanol and acetic acid) at a temperature between ambient temperature and reflux (preferably solvent reflux) for a period between 5 minutes and 24 hours (preferably 1 hour).

The reaction of acids of formula II with amines of formula IX to make amides of formula I may employ a range of amide coupling conditions known to those skilled in the art, including preparation of the corresponding acid chloride or acyl imidazole, or directly from the acid employing, EDCI, CU, HATU or other coupling reagents known to those skilled in the art, preferably preparing the acid chloride by treatment of the carboxylic acid II with oxalyl chloride in halogenated solvent such as dichloromethane or dichloroethane (preferably dichloroethane) optionally with a catalyst such as pyridine, dimethylaminopyridine, imidazole or an amide (preferentially dimethylformamide) at a range of temperatures between −78° C. and solvent reflux (preferably at 0° C.) and followed by warming to ambient temperature for a period between 5 minutes and 24 hours (preferably 2 hours) followed by removal of the volatiles by concentration under vacuum with the aid of a rotary evaporator. The resulting acid chloride is then dissolved in a halogenated solvent such as dichloromethane or dichloroethane (preferentially dichloroethane) and combined with amine IX in a solvent, preferentially a halogenated solvent such as dichloromethane or dichloroethane (preferentially dichloroethane) in the presence of a base such as a hydroxide, a carbonate, a pyridine, an amidine, or a teriary amine (preferentially triethylamine). Preferentially the acid chloride of acid II is added as a dichloroethane solution to a stirred solution of the amine IX in combination with the base (preferentially triethylamine) at a temperature between −78° C. and solvent reflux (preferentially 0° C.) and stirred at ambient temperature for a period between 1 minute to 24 hours, (preferentially 1 hour) before working up in the usual manner to provide amides of formula I.

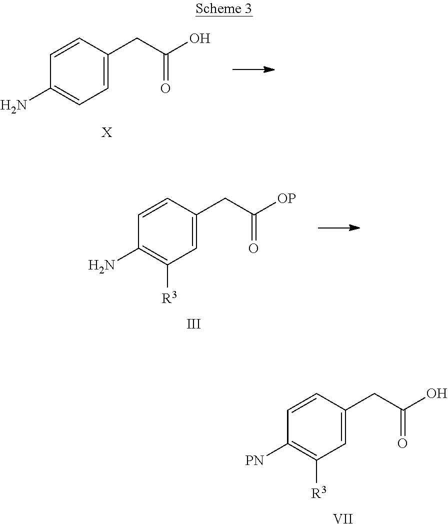

According to reaction sequence depicted in Scheme 3, the desired compounds wherein R³ is as described in the Summary and wherein the acid P-group is selected from a range of suitable groups including but not limited to methyl, ethyl, isopropyl, tert-butyl, allyl, and benzyl (preferably methyl) and wherein the nitrogen P-group is selected from a range of suitable protecting group including but not limited to are tert-butyl carbamate, benzylcarbamate (preferably tert-butylcarbamate). It is recognized by those skilled in the art that electrophilic aromatic substitution reactions can be used to introduce substituents ortho to an electron-donating group such as the nitrogen in (4-aminophenyl)acetic acid and those substituents can be interconverted with a variety of other substituents including those as defined for R³ using methods known to those skilled in the art and found described extensively throughout the synthetic chemical literature. For the instance where R³ is equal to chlorine the amine may be first derivatized with an electron withdrawing group such as acetate or trifluoroactetate (preferably acetate) by the reaction with the corresponding anhydride or acid chloride (preferably acetic anhydride) and the resulting amide reacted with a chlorinating agent such as sulfuryl chloride (SO₂Cl₂) or calcium hypochlorite (preferably calcium hypochlorite) in a mixture of ethanol, acetic acid and water at a temperature between 0° C. and solvent reflux (preferably ambient temperature) for between 5 minutes and 24 hours (preferably 1 hour). After workup and isolation of the intermediate chloroamide the amide can be removed and the ester P-group formed by reaction of the amide with an acid (preferably concentrated hydrochloric acid) in an alcoholic solvent of the corresponding P-groups such as methanol, ethanol or isopropanol (preferably methanol) at a temperature between room temperature and solvent reflux (preferably solvent reflux) for a period between 5 minutes and 24 hours (preferably 1.5 hours) to obtain after workup and isolation compounds of formula III. Other R³ groups of the invention may be obtained by the corresponding acylation and appropriate functional group manipulation or by nitration followed by reduction of the nitro group to an aniline and conversion of the aniline to R³ groups of the invention via a diazonium intermediate and modification employing transition metal catalysis as described in the chemical literature.

Compounds of formula VII may be obtained from amines of formula III by conversion of the nitrogen to a suitably protected form such as tert-butylcarbamate or benzylcarbamate (preferably tert-butylcarbamate) employing a suitable reagent such as benzylchloroformate or tert-butylcarbonic anhydride. The protected acid is then revealed by a range of ester cleaving methods known to those skilled in the art including acid or base treatment (preferably reaction with lithium hydroxide). For compounds of formula III where P is a lower alkyl group such as methyl, ethyl, isopropyl (preferably methyl) the ester may be cleaved by treatment with an alkali metal hydroxide, or other methods known to those skilled in the art for cleaving esters to acids (preferably treatment of the ester as a solution in tetrahydrofuran and methanol with an aqueous lithium hydroxide solution, preferably 1 molar) at a temperature between 0° C. and solvent reflux (preferably room temperature) for between 5 minutes and 24 hours (preferably 1 hour) followed by acidic workup to afford the carboxylic acid VII. Compounds of Formula VII are useful for preparing the compounds of the invention as described for Formula I in Scheme 2.

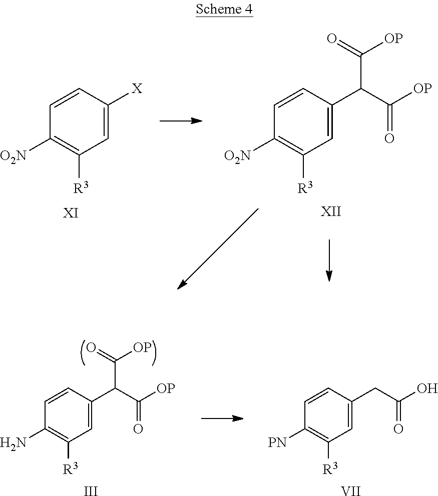

According to the reaction sequence depicted in Scheme 4, the desired compounds wherein R³ is as described in the Summary and wherein the acid P-group is selected from a range of suitable groups including but not limited to methyl, ethyl, isopropyl, tert-butyl, allyl, and benzyl (preferably methyl) and wherein the nitrogen P-group is selected from a range of suitable protecting groups including but not limited to tert-butyl carbamate, and benzylcarbamate (preferably tert-butylcarbamate). Nitrobenzene compounds of Formula XI where R³ is as described in the Summary and X is a leaving group such as fluoride, chloride, bromide, iodide, or trifluoromethylsulfonate (preferably fluoride) may be reacted with the sodium or potassium (preferably sodium) enolate of a malonate such as dimethyl, diethyl, diisopropyl, di-tert-butyl, ethyl-tert-butyl, or tert-butyl-cyano (preferably diethyl malonate) in a polar aprotic solvent such as tetrahydrofuran, dimethylformamide, dimethylacetamide or N-methylpyrrolidone (preferably dimethylformamide) at a temperature between room temperature and solvent reflux (preferably 120° C.) for a duration of between 5 minutes and 24 hours (preferably 3 hours) to provide compounds of Formula XII.

Nitrobenzenes of formula XII may be converted to amines of formula III by reduction employing a variety of methods known to those skilled in the art such as treating a solution of the nitro group and a catalyst such as palladium on carbon (preferably 10% palladium on carbon) in an alcoholic solvent (preferably ethanol) with a hydrogen source (preferably hydrogen gas) at a temperature between 0° C. and solvent reflux (preferably ambient temperature) and a pressure between 1 and 10 atmospheres (preferably 3 atmospheres). Nitrobenzenes of formula XII may also be converted to amines of formula III by reduction with a reducing metal (preferably iron filings) in a solvent (preferably ethanol and acetic acid) at a temperature between ambient temperature and reflux (preferably solvent reflux) for a period between 5 minutes and 24 hours (preferably 1 hour).

Phenylmalonates of Formula XII may be converted to the phenylacetic esters of Formula III by first treating the malonate ester with acid (preferably hydrochloric acid) in an alcoholic solvent such as methanol or ethanol (preferably methanol) at a temperature between 0° C. and solvent reflux (preferably room temperature) for a period between 1 hour and 24 hours (preferably 3 hours) to afford, after reduction of the nitro group as described above, phenylacetates of formula III. Protected amino phenylacetates of Formula VII may be obtained from amines of Formula III by reacting as described above for Scheme 3. Compounds of Formula VII may also be obtained where the nitrogen is protected as its corresponding nitro analog from nitrophenylmalonates of Formula XII by treatment with an acidic aqueous solution (preferably 6 M hydrochloric acid) at a temperature between room temperature and solvent reflux (preferably solvent reflux) for a duration between 1 hour and 24 hours (preferably 3.5 hours). Compounds of Formula I may be prepared from compounds of Formula VII as described above for Scheme 2.

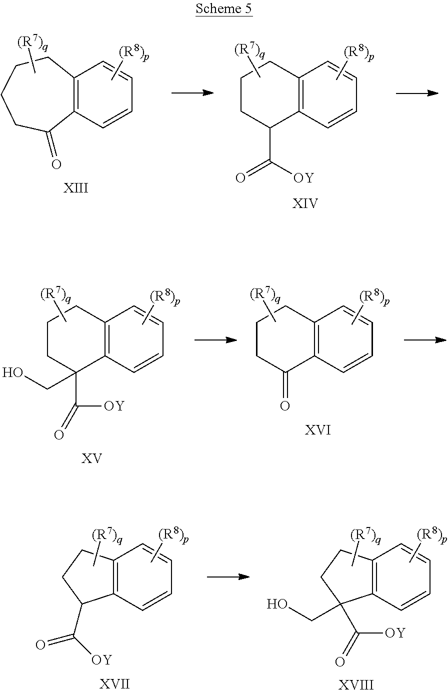

According to the reaction sequence depicted in Scheme 5, the desired compounds XV and XVIII corresponding to certain compounds of Formula VI may be prepared wherein R⁷, R⁸, Y, p and q are as described in the Summary. Benzocycloheptanones of Formula XIII and benzocyclohexanones of Formula XVI may be purchased from commercial sources, are known in the literature or can be prepared according methods known to those skilled in the art, such as Freidel-Crafts acylation or nuceophilic addition of a metalloaryl species with glutaric or succinic anhydride, ketone reduction, and cyclization of the activated acyl species to provide the cyclic ketones of Formula XIII or XVI. Employing these compounds in an oxidative ring contraction reaction (preferably treatment with lead tetraacetate) in the presence of a Lewis acid (preferably boron trifluoride etherate) in a solvent system containing an alcohol (preferably ethanol in toluene) at a temperature between 0° C. and solvent reflux (preferably room temperature) for between 1 hour and 7 days (preferably 3 days) affords the compounds of Formula XIV or Formula XVII. Compounds of Formula XV or Formula XVIII may correspondingly be prepared from compounds of Formula XIV or XVII by treatment with a base (preferably lithium diisopropylamide) in a polar solvent (preferably tetrahydrofuran) at a temperature between −100° C. and room temperature (preferably −78° C.) for a period between 5 minutes and 5 hours (preferably 1 hour) before being treated with a source of formaldehyde (preferably paraformaldehyde) at a temperature between −100° C. and room temperature (preferably 0° C.) for a period between 5 minutes and 5 hours (preferably 30 minutes) before an extractive workup to afford the desired compounds corresponding to Formula VI which may be used to prepare the compounds of Formula I of the invention as described above for Scheme 1 and Scheme 2.

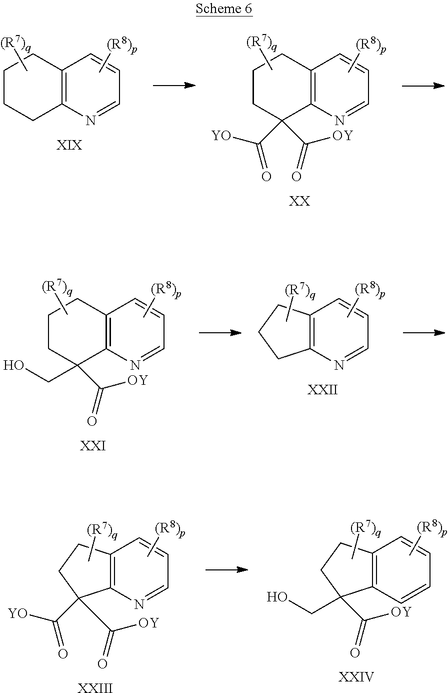

According to the reaction sequence depicted in Scheme 6, the desired compounds XXI and XXIV corresponding to certain compounds of Formula VI may be prepared wherein R⁷, R⁸, Y, p and q are as described in the Summary. Compounds of Formula XIX and Formula XXII may be purchased from commercial sources, are known in the literature or can be prepared by various methods known to those skilled in the synthetic arts such as constructing the corresponding cyclohexanone or cyclopentanone and annulating the pyridine ring onto the cyclohexane or cyclopentane system. Compounds of Formula XX and XXIII may be correspondingly prepared from XIX and XXII by treatment with a base (preferably tert-butyllithium) in a polar aprotic solvent system (preferably 2-methyltetrahydrofuran and tetramethylethylenediamine) at a temperature between −100° C. and room temperature (preferably −78° C.) before being added to a solution of a carboalkoxylating agent (preferably ethyl cyanoformate) in a polar aprotic solvent (preferably 2-methyltetrahydrofuran) at a temperature between −100° C. and room temperature (preferably −78° C. and allowing to warm to room temperature after addition) for a period between 5 minutes and 2 hours (preferably 20 minutes) to afford the desired compounds after extractive workup. Compounds of Formula XXI and Formula XXIV may be obtained correspondingly from compounds of Formula XX and Formula XXIII by treatment with a reducing agent (preferably lithium tri-tert-butoxyaluminumhydride) in a polar aprotic solvent (preferably tetrahydrofuran) at a temperature between 0° C. and solvent reflux (preferably starting at room temperature and warming to solvent reflux) for a period between 5 minutes and 5 hours (preferably 30 minutes) to afford the desired compounds after extractive workup. Compounds of Formula XXI and XXIV may also be obtained correspondingly from compounds of Formula XX and Formula XXIII by monodecarboalkoxylation by treatment with a nucleophile such as sodium ethoxide or sodium hydroxide (preferably sodium hydroxide) in a polar solvent (preferably ethanol) at a temperature between 0° C. and solvent reflux (preferably room temperature) for a period between 1 hour and 24 hours (preferably 12 hours) to afford the monoester after extractive workup, which is then converted to the compounds of Formula XXI and Formula XXIV by reaction with a base (preferably 1,8-diazabicyclo[5.4.0]-undec-7-ene, DBU) and a source of formaldehyde (preferably paraformaldehyde) in a polar solvent (preferably dioxane) at a temperature between 0° C. and 200° C. (preferably room temperature) for a duration between 5 minutes and 24 hours (preferably 1 hour) to afford the desired compounds of Formula XXI and XXIV after extractive workup.

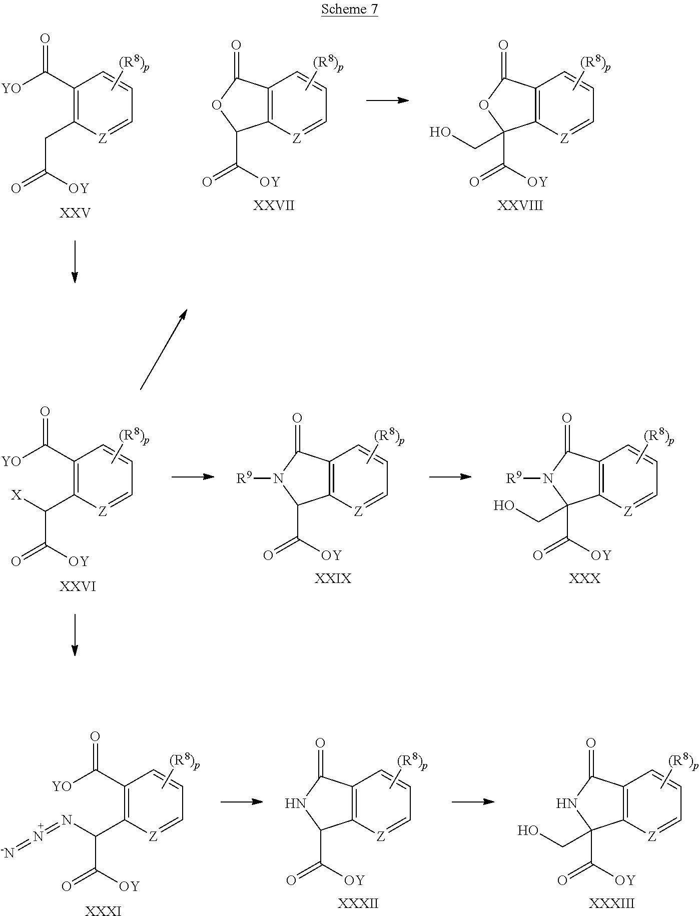

According to the reaction sequence depicted in Scheme 7, the desired compounds XXVIII and XXX corresponding to certain compounds of Formula VI may be prepared wherein R⁸, R⁹, Y, and p are as described in the Summary and Z is equal to CH or N. Compounds of Formula XXV may be purchased from commercial sources, are known in the literature, or can be prepared by various methods known to those skilled in the synthetic arts. Compounds of Formula XXVI wherein X is equal to a group able to be displaced by a nucleophile such as fluorine, chlorine, bromine, iodine, alkylsulfonate (preferably bromine) may be prepared by reducing the corresponding ketone and converting the resulting alcohol into one of the X groups above or by introducing the X group directly (preferably bromide) by reacting the diester of Formula XXV with an electrophilic source of bromine (preferably N-bromosuccinimide) with a catalytic amount of a radical source (preferably alpha,alpha′-azoisobutyronitrile) in a suitable solvent such as carbon tetrachloride or dichloroethane (preferably carbon tetrachloride) at a temperature between room temperature and solvent reflux (preferably 80° C.) for a period of time between 1 hour and 10 days (preferably 1 day) to afford the compound of Formula XXVI after extractive workup. Compounds of Formula XXVII may be obtained from compounds of Formula XXVI by reaction with a source carboxylate salt (preferably potassium acetate) in a polar solvent (preferably dimethylacetamide) at a temperature between room temperature and solvent reflux (preferably 85° C.) for a period of time between 1 hour and 48 hours (preferably 16 hours) then after extractive workup the product was treated with an acidic alcoholic solution (preferably hydrochloric acid in ethanol) at a temperature between room temperature and solvent reflux (preferably 70° C.) for a period of time between 1 and 24 hours (preferably 12 hours) to afford compounds of Formula XXVII after extractive workup. Compounds of Formula XXVIII can be obtained from compounds of Formula XXVII by treatment with a formaldehyde source (preferably paraformaldehyde) and an amine base (preferably 1,8-diazabicyclo[5.4.0]-undec-7-ene) in a polar solvent (preferably dioxane) at a temperature between 0° C. and solvent reflux (preferably room temperature) for a period between 5 minutes and 24 hours (preferably 1.5 hours) to give the desired compounds of Formula XXVIII after extractive workup. Additionally, compounds of Formula XXIX may be obtained from compounds of Formula XXVI by treatment with an alkyl amine in a polar solvent (preferably acetonitrile) at a temperature between −78° C. and solvent reflux (preferably 0° C.) for a period between 1 hour to 24 hours (preferably 18 hours) and then a temperature between 0° C. and solvent reflux (preferably 40° C.) for a period between 1 hour to 24 hours (preferably 2.5 hours) to afford the compounds of Formula XXIX after extractive workup. Compounds of Formula XXX may be obtained from compounds of Formula XXIX by treatment with a formaldehyde source (preferably paraformaldehyde) and an amine base (preferably 1,8-diazabicyclo[5.4.0]-undec-7-ene) in a polar solvent (preferably dioxane) at a temperature between 0° C. and solvent reflux (preferably room temperature) for a period between 5 minutes and 24 hours (preferably 1 hour) to give the desired compounds of Formula XXX after extractive workup. Additionally, compounds of Formula XXXI may be obtained from compounds of Formula XXVI by treatment with an azide (preferably sodium azide) in a polar solvent (preferably acetonitrile) at a temperature between −78° C. and solvent reflux (preferably room temperature) for a period between 1 hour to 48 hours (preferably 24 hours) to afford the compounds of Formula XXXI after concentration of the reaction filtrate. Compounds of Formula XXXII may be obtained from compounds of Formula XXXI by reducing agents such as treatment with a hydrogen source (preferably 1,4-cyclohexadiene) in the presence of a catalyst (preferably 10% palladium on carbon) in a polar solvent (preferably ethanol) at a temperature between room temperature and solvent reflux (preferably 70° C.) for a period between 1 to 24 hours (preferably 2 hours) to afford compounds of Formula XXXII after filtration and concentration. Compounds of Formula XXXIII may be obtained from compounds of Formula XXXII by treatment with a formaldehyde source (preferably paraformaldehyde) and an amine base (preferably 1,8-diazabicyclo[5.4.0]-undec-7-ene) in a polar solvent (preferably dioxane) at a temperature between 0° C. and solvent reflux (preferably room temperature) for a period between 5 minutes and 24 hours (preferably 1 hour) to give the desired compounds of Formula XXXIII.

As is readily apparent to one skilled in the art, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBZ) and 9-fluorenylmethyleneoxycarbonyl (Fmoc). Similarly, a “hydroxy-protecting group” refers to a substituent of a hydroxy group that blocks or protects the hydroxy functionality. Suitable hydroxyl-protecting groups (O-Pg) include for example, allyl, acetyl, silyl, benzyl, para-methoxybenzyl, trityl, tert-butyldimethylsilyl, benzyloxymethylene and the like. The need for such protection is readily determined by one skilled in the art. For a general description of protecting groups and their use, see P. G. M. Wuts, T. W. Greene, Greene's Protective Groups in Organic Synthesis, John Wiley & Sons, New York, 2006.

As noted above, some of the compounds of this invention are acidic and they form salts with pharmaceutically acceptable cations. Some of the compounds of this invention are basic and form salts with pharmaceutically acceptable anions. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds are obtained in crystalline form according to procedures known in the art, such as by dissolution in an appropriate solvent(s) such as ethanol, hexanes or water/ethanol mixtures

As noted above, some of the compounds exist as isomers. These isomeric mixtures can be separated into their individual isomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

Certain compounds of the present invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast cooling. The presence of polymorphs may be determined by solid probe NMR spectroscopy, IR spectroscopy, differential scanning calorimetry, powder X-ray diffraction or such other techniques.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), AstraZeneca Pharmaceuticals (London, England), Mallinckrodt Baker (Phillipsburg N.J.), EMD (Gibbstown, N.J.), Ark Pharm Inc (Libertyville, Ill.), Matrix Scientific (Columbia, S.C.), and Combi-Blocks (San Diego, Calif.).

### General Experimental Procedures

Reactions were performed in air or, when oxygen- or moisture-sensitive reagents or intermediates were employed, under an inert atmosphere (nitrogen or argon). When appropriate, reaction apparatuses were dried under dynamic vacuum using a heat gun, and anhydrous solvents (Sure-Seal™ products from Aldrich Chemical Company, Milwaukee, Wis. or DriSolv™ products from EMD Chemicals, Gibbstown, N.J.) were employed. Commercial solvents and reagents were used without further purification. When indicated, reactions were heated by microwave irradiation using Biotage Initiator or Personal Chemistry Emuys Optimizer microwaves. Reaction progress was monitored using thin layer chromatography (TLC), liquid chromatography-mass spectrometry (LCMS), high performance liquid chromatography (HPLC), and/or gas chromatography-mass spectrometry (GCMS) analyses. TLC was performed on pre-coated silica gel plates with a fluorescence indicator (254 nm exitation wavelength) and visualized under UV light and/or with I₂, KMnO₄, CoCl₂, phosphomolybdic acid, and/or ceric ammonium molybdate stains. LCMS data were acquired on an Agilent 1100 Series instrument with a Leap Technologies autosampler, Gemini C18 columns, MeCN/water gradients, and either TFA, formic acid, or ammonium hydroxide modifiers.

The column eluent was analyzed using Waters ZQ mass spectrometer scanning in both positive and negative ion modes from 100 to 1200 Da. Other similar instruments were also used. HPLC data were acquired on an Agilent 1100 Series instrument using Gemini or XBridge C18 columns, MeCN/water gradients, and either TFA or ammonium hydroxide modifiers. GCMS data were acquired using a Hewlett Packard 6890 oven with an HP 6890 injector, HP-1 column (12 m×0.2 mm×0.33 μm), and helium carrier gas. The sample was analyzed on an HP 5973 mass selective detector scanning from 50 to 550 Da using electron ionization. Purifications were performed by medium performance liquid chromatography (MPLC) using Isco CombiFlash Companion, AnaLogix IntelliFlash 280, Biotage SP1, or Biotage Isolera One instruments and pre-packed Isco RediSep or Biotage Snap silica cartridges. Chiral purifications were performed by chiral supercritical fluid chromatography (SFC) using Berger or Thar instruments; ChiralPAK-AD, -AS, -IC, Chiralcel-OD, or -OJ columns; and CO₂ mixtures with MeOH, EtOH, iPrOH, or MeCN, alone or modified using TFA or iPrNH₂. UV detection was used to trigger fraction collection.

Mass spectrometry data are reported from LCMS analyses. Nuclear magnetic resonance (NMR) spectra were recorded on 400 and 500 MHz Varian spectrometers. Chemical shifts are expressed in parts per million (ppm, δ) referenced to the deuterated solvent residual peaks. Analytical SFC data were acquired on a Berger analytical instrument as described above. Optical rotation data were acquired on a PerkinElmer model 343 polarimeter using a 1 dm cell.

Concentration in vacuo refers to evaporation of solvent under reduced pressure using a rotary evaporator.

Unless otherwise noted, chemical reactions were performed at room temperature (about 23 degrees Celsius).

### Preparation 1: (S)- and (R)-Ethyl 1-(hydroxymethyl)-2-methyl-3-oxoisoindoline-1-carboxylate

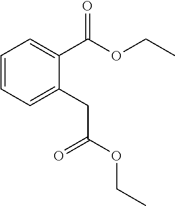 [SMILES: CCOC(=O)Cc1ccccc1C(=O)OCC]

### Step A: Ethyl 2-(2-ethoxy-2-oxoethyl)benzoate

Thionyl chloride (5.0 mL, 69 mmol) was added to a stirred solution of homophthalic acid (6.11 g, 33.9 mmol) in EtOH (240 mL), portion-wise, at room temperature. The resulting solution was heated in an aluminum block at 75° C. for 24 hours. The reaction mixture was then concentrated by rotary evaporation and partitioned between EtOAc and sat. aq. NaHCO₃. The organic layer was washed with brine and dried over Na₂SO₄. Rotary evaporation of the organic layer provided the title compound (7.71 g, 96% yield) as a clear, yellow-orange liquid. LCMS (ESI) m/z: 191.0 [M-EtOH+H] (100%), 259.0 [M+Na] (85%). GCMS (EI) m/z: 135 [M-CO₂Et-Et+H] (100%), 190 [M-EtOH] (67%). ¹H NMR (400 MHz, CDCl₃) δ 1.26 (t, J=7.1 Hz, 3H), 1.38 (t, J=7.1 Hz, 3H), 4.02 (s, 2H), 4.17 (q, J=7.1 Hz, 2H), 4.34 (q, J=7.1 Hz, 2H), 7.26 (ddd, J=7.7, 1.3, 0.5 Hz, 1H), 7.37 (td, J=7.6, 1.4 Hz, 1H), 7.48 (td, J=7.4, 1.5 Hz, 1H), 8.03 (dd, J=7.8, 1.4 Hz, 1H).

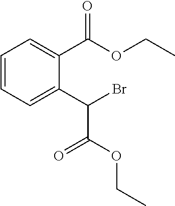 [SMILES: CCOC(=O)c1ccccc1C(Br)C(=O)OCC]

### Step B: (±)-Ethyl 2-(1-Bromo-2-ethoxy-2-oxoethyl)benzoate

A solution of ethyl 2-(2-ethoxy-2-oxoethyl)benzoate (7.00 g, 29.6 mmol) in carbon tetrachloride (148 mL) was treated with N-bromosuccinimide (5.33 g, 29.9 mmol). α,α′-Azoisobutyronitrile (487 mg, 2.96 mmol) was then added to the mixture. The reaction mixture was heated at 80° C. for 20 hours, cooled down to room temperature, and then diluted with heptane. The organic layer was washed 3 times with water and dried over MgSO₄. Filtration and removal of solvent by rotary evaporation gave a clear, pale yellow residue, which was purified by MPLC (gradient from pure heptane to 7:3 EtOAc/heptane) to afford the title compound (8.30 g, 89% yield) as a clear, colorless liquid. LCMS (ESI) m/z: 314.8 [M+H] (97%), 316.7 [M+2+H] (100%). ¹H NMR (400 MHz, CDCl₃) δ 1.29 (t, J=7.1 Hz, 3H), 1.41 (t, J=7.1 Hz, 3H), 4.23 (dq, J=10.9, 7.1 Hz, 1H), 4.28 (dq, J=10.7, 7.1 Hz, 1H), 4.40 (q, J=7.0 Hz, 2H), 6.59 (s, 1H), 7.41 (td, J=7.7, 1.1 Hz, 1H), 7.58 (td, J=7.7, 1.3 Hz, 1H), 7.89 (dd, J=7.9, 0.7 Hz, 1H), 7.97 (dd, J=7.8, 1.2 Hz, 1H).

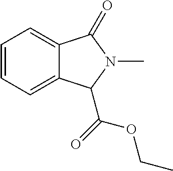 [SMILES: CCOC(=O)C1c2ccccc2C(=O)N1C]

### Step C: (±)-Ethyl 2-methyl-3-oxoisoindoline-1-carboxylate

A solution of (±)-ethyl 2-(1-bromo-2-ethoxy-2-oxoethyl)benzoate (7.30 g, 23.2 mmol) in MeCN (53 mL) was cooled to 0° C. and then treated with 2.0 M methylamine in THF (34.7 mL, 69.5 mmol). The reaction mixture was stirred at room temperature for 18 hours and then at 40° C. for 2.5 hours. A white precipitate that formed during the reaction was removed by filtration and rinsed with EtOAc. The filtrate was evaporated, and the residue was partitioned between EtOAc and water. The organic layer was washed with water and brine, dried over MgSO₄, filtered, and concentrated by rotary evaporation. The residue obtained was purified by MPLC (gradient from pure heptane to 7:3 EtOAc/heptane) to afford the title compound as a white, crystalline solid (2.89 g, 57% yield). LCMS (ESI) m/z: 220.1 [M+H] (100%). ¹H NMR (400 MHz, CDCl₃) δ 1.32 (t, J=7.1 Hz, 3H), 3.21 (s, 3H), 4.23 (dq, J=10.7, 7.1 Hz, 1H), 4.33 (dq, J=10.7, 7.1 Hz, 1H), 5.07 (s, 1H), 7.51 (td, J=7.3, 1.4 Hz, 1H), 7.56 (td, J=7.4, 1.5 Hz, 1H), 7.60-7.63 (m, 1H), 7.84-7.87 (m, 1H).

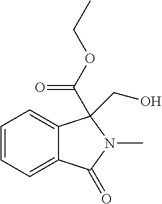 [SMILES: CCOC(=O)C1(CO)c2ccccc2C(=O)N1C]

### Step D: (±)-Ethyl 1-(hydroxymethyl)-2-methyl-3-oxoisoindoline-1-carboxylate

To a solution of (±)-ethyl 2-methyl-3-oxoisoindoline-1-carboxylate (5.09 g, 23.2 mmol) in 1,4-dioxane (46 mL) was added paraformaldehyde (1.51 g, 50.3 mmol) and DBU (694 μL, 4.64 mmol). The resulting heterogeneous mixture was stirred vigorously at room temperature for 1 hours, and then the solvent was removed by rotary evaporation. The residue was dissolved in EtOAc, washed 2 times with water, and washed with brine. The organic layer was dried over MgSO₄ and concentrated by rotary evaporation. The residue was purified by MPLC (gradient from 3:17 EtOAc/heptane to pure EtOAc) to afford the title compound (4.30 g, 74.3% yield). LCMS (ESI) m/z: 250.1 [M+H] (100%). ¹H NMR (400 MHz, CDCl₃) δ 1.22 (t, J=7.1 Hz, 3H), 2.59 (t, J=6.9 Hz, 1H), 3.18 (s, 3H), 4.07-4.36 (m, 4H), 7.47-7.51 (m, 1H), 7.54-7.60 (m, 2H), 7.75-7.84 (m, 1H).

### Step E: Ethyl (1S)-1-(Hydroxymethyl)-2-methyl-3-oxoisoindoline-1-carboxylate

(±)-Ethyl 1-(hydroxymethyl)-2-methyl-3-oxoisoindoline-1-carboxylate (57.5 g, 231 mmol) was purified by chiral SFC (Chiralpak AS-H 50×250 mm column, 90:10 CO₂/MeOH) to give the title compound (25.57 g). By analytical chiral SFC (Chiralpak AS-H 4.6×250 mm column, 90:10 CO₂/MeOH, 2.5 mL/min.), this product was observed to have t_{R}=2.83 min. [α]_{D}=+114° (MeOH, c=2.5).

### Step F: Ethyl (1R)-1-(Hydroxymethyl)-2-methyl-3-oxoisoindoline-1-carboxylate

(±)-Ethyl 1-(hydroxymethyl)-2-methyl-3-oxoisoindoline-1-carboxylate (57.5 g, 231 mmol) was purified by chiral SFC (Chiralpak AS-H 30×250 mm column, 90:10 CO₂/MeOH) to give the title compound (26.21 g). By analytical chiral SFC (Chiralpak AS-H 4.6×250 mm column, 90:10 CO₂/MeOH, 2.5 mL/min.), this product was observed to have t_{R}=4.21 min. [α]_{D}=−123° (MeOH, c=2.0).

### Preparation 2: (±)-Ethyl 1-(Hydroxymethyl)-2-ethyl-3-oxoisoindoline-1-carboxylate

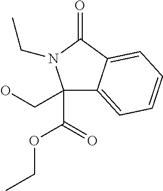 [SMILES: CCOC(=O)C1(CO)c2ccccc2C(=O)N1CC]

The title compound was prepared following the general route of Preparation 1 using ethyl amine. LCMS (ESI) m/z: 264.1 [M+H] (100%). ¹H NMR (400 MHz, CDCl₃) δ 1.19 (t, J=7.1 Hz, 3H), 1.35 (t, J=7.2 Hz, 3H), 2.31 (t, J=6.9 Hz, 1H), 3.46 (dq, J=14.3, 7.1 Hz, 1H), 3.83 (dq, J=14.2, 7.2 Hz, 1H), 4.12 (dq, J=10.7, 7.1 Hz, 1H), 4.14 (dd, J=11.8, 7.0 Hz, 1H), 4.24 (dq, J=10.7, 7.1 Hz, 1H), 4.28 (dd, J=11.9, 6.8 Hz, 1H), 7.49-7.60 (m, 3H), 7.85 (dt, J=7.0, 1.3 Hz, 1H).

### Preparation 3: (±)-Ethyl 1-(Hydroxymethyl)-2-isopropyl-3-oxoisoindoline-1-carboxylate

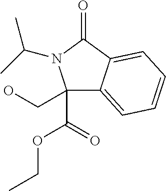 [SMILES: CCOC(=O)C1(CO)c2ccccc2C(=O)N1C(C)C]

The title compound was prepared following the general route of Preparation 1 using isopropylamine. LCMS (ESI) m/z: 278.1 [M+H] (100%). ¹H NMR (400 MHz, CDCl₃) δ 1.22 (t, J=7.1 Hz, 3H), 1.55 (d, J=6.7 Hz, 3H), 1.65 (d, J=6.7 Hz, 3H), 2.18 (t, J=7.0 Hz, 1H), 3.83 (spt, J=6.8 Hz, 1H), 4.15 (dq, J=10.8, 7.1 Hz, 1H), 4.14-4.20 (m, 1H), 4.17-4.23 (m, 1H), 4.26 (dq, J=10.8, 7.2 Hz, 1H), 7.48-7.58 (m, 3H), 7.80-7.84 (m, 1H).

### Preparation 4: (±)-Ethyl 1-(Hydroxymethyl)-2-(2,2,2-trifluoroethyl)-3-oxoisoindoline-1-carboxylate

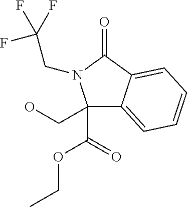 [SMILES: CCOC(=O)C1(CO)c2ccccc2C(=O)N1CC(F)(F)F]

The title compound was prepared following the general route of Preparation 1 using 2,2,2-trifluoroethyl amine. LCMS (ESI) m/z: 318.1 [M+H] (100%). ¹H NMR (400 MHz, CDCl₃) δ 1.19 (t, J=7.1 Hz, 3H), 2.21 (dd, J=7.2, 6.4 Hz, 1H), 4.09-4.28 (m, 4H), 4.40-4.56 (m, 2H), 7.54-7.60 (m, 2H), 7.61-7.67 (m, 1H), 7.89-7.95 (m, 1H).

### Preparation 5: (±)-Ethyl 1-(Hydroxymethyl)-2-(2,4-dimethoxybenzyl)-3-oxoisoindoline-1-carboxylate

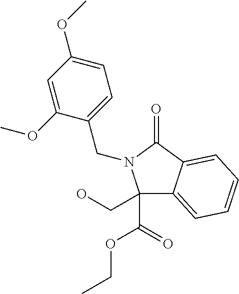 [SMILES: CCOC(=O)C1(CO)c2ccccc2C(=O)N1Cc1ccc(OC)cc1OC]

The title compound was prepared following the general route of Preparation 1 using 2,4-dimethoxybenzyl amine. ¹H NMR (400 MHz, CDCl₃) δ 1.13 (t, J=7.1 Hz, 3H), 1.82 (dd, J=8.7, 6.3 Hz, 1H), 3.79 (s, 3H), 3.84 (s, 3H), 3.97 (dd, J=12.2, 6.3 Hz, 1H), 4.07 (q, J=7.1 Hz, 2H), 4.18 (dd, J=12.2, 8.7 Hz, 1H), 4.61 (d, J=15.3 Hz, 1H), 5.04 (d, J=15.3 Hz, 1H), 6.45-6.49 (m, 2H), 7.43 (d, J=8.0 Hz, 1H), 7.50-7.59 (m, 3H), 7.89 (dt, J=7.2, 1.1 Hz, 1H).

### Preparation 6: (+)- and (−)-Ethyl 1-(Hydroxymethyl)-3-oxoisoindoline-1-carboxylate

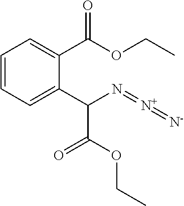 [SMILES: CCOC(=O)c1ccccc1C(N=[N+]=[N-])C(=O)OCC]

### Step A: (±)-Ethyl 2-(1-azido-2-ethoxy-2-oxoethyl)benzoate

To a solution of (±)-ethyl 2-(1-bromo-2-ethoxy-2-oxoethyl)benzoate (1.99 g, 6.30 mmol) in MeCN (10. mL) was added sodium azide (699 mg, 10.8 mmol). The resulting fine white suspension was stirred vigorously at room temperature for 22 hours. The reaction mixture was diluted with MTBE (ca. 15 mL) and then filtered. After washing the solids with MTBE, the solvent was removed from the filtrate by rotary evaporation to afford the title compound as a pale yellow oil (1.73 g, 98.9% yield). LCMS (ESI) m/z: 300.0 [M+Na] (100%). ¹H NMR (400 MHz, CDCl₃) δ 1.27 (t, J=7.1 Hz, 3H), 1.41 (t, J=7.2 Hz, 3H), 4.22 (dq, J=10.7, 7.1 Hz, 1H), 4.28 (dq, J=10.7, 7.1 Hz, 1H), 4.39 (q, J=7.2 Hz, 2H), 6.21 (s, 1H), 7.45 (ddd, J=7.8, 7.0, 1.8 Hz, 1H), 7.53-7.61 (m, 2H), 8.04 (dd, J=7.8, 1.3 Hz, 1H).

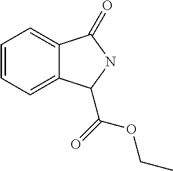

### Step B: (±)-Ethyl 3-oxoisoindoline-1-carboxylate

To a solution of (±)-ethyl 2-(1-azido-2-ethoxy-2-oxoethyl)benzoate (1.72 g, 6.21 mmol) in EtOH (41 mL) was added 1,4-cyclohexadiene (15 mL) and 10% Pd/C (1.29 g, 0.61 mmol, 50% wet). The reaction mixture was heated briefly at reflux and then for two hours in an aluminum block at 70° C. After the reaction mixture cooled to room temperature, it was filtered through Celite, which was washed with EtOAc. The solvent was removed from the filtrate by rotary evaporation to afford a pale yellow solid. This crude material was briefly re-pulped in boiling EtOAc (10. mL), cooled to room temperature, and filtered to afford the title compound (909 mg, 71.3% yield) as a white, crystalline solid. LCMS (ESI) m/z: 206.0 [M+H] (100%). ¹H NMR (400 MHz, CDCl₃) δ 1.33 (t, J=7.1 Hz, 3H), 4.26 (dq, J=10.8, 7.1 Hz, 0H), 4.30 (dq, J=10.8, 7.1 Hz, 1H), 5.25 (d, J=0.6 Hz, 1H), 6.66 (br. s., 1H), 7.54 (m, J=7.5, 7.5, 1.1, 0.7 Hz, 1H), 7.62 (td, J=7.5, 1.2 Hz, 1H), 7.73 (dq, J=7.6, 0.9 Hz, 1H), 7.87 (dt, J=7.4, 1.0 Hz, 1H).

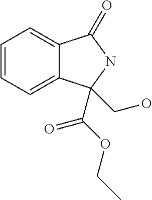 [SMILES: CCOC(=O)C1(CO)NC(=O)c2ccccc21]

### Step D: (±)-Ethyl 1-(hydroxymethyl)-3-oxoisoindoline-1-carboxylate

DBU (8.3 μL, 0.055 mmol) was added to a suspension of (±)-ethyl 3-oxoisoindoline-1-carboxylate (56.4 mg, 0.275 mmol) and paraformaldehyde (8.5 mg, 0.28 mmol) in 1,4-dioxane (0.55 mL). The reaction mixture was heated for 15 min. in an aluminum block at 60° C., upon which it became a foggy solution. The volatile components of the reaction mixture were removed by rotary evaporation, and the residue was purified by MPLC (gradient from 3:2 EtOAc/heptane to pure EtOAc) to afford the title compound (32.1 mg, 49.6% yield) as a clear, colorless glass. LCMS (ESI) m/z: 236.1 [M+H] (100%). ¹H NMR (400 MHz, CDCl₃) δ 1.28 (t, J=7.1 Hz, 3H), 3.35 (t, J=6.4 Hz, 1H), 3.70 (dd, J=10.9, 6.3 Hz, 1H), 4.23 (dq, J=10.9, 7.1 Hz, 1H), 4.28 (dq, J=10.8, 7.2 Hz, 1H), 4.48 (dd, J=11.0, 6.7 Hz, 1H), 7.44 (br. s., 1H), 7.54 (td, J=7.4, 0.6 Hz, 1H), 7.61 (td, J=7.4, 1.0 Hz, 1H), 7.68 (d, J=7.4 Hz, 1H), 7.85 (d, J=7.4 Hz, 1H).

### Step E: (−)-Ethyl 1-(hydroxymethyl)-3-oxoisoindoline-1-carboxylate

(±)-Ethyl 1-(hydroxymethyl)-3-oxoisoindoline-1-carboxylate (32.1 mg, 0.136 mmol) was purified by chiral SFC (Chiralpak AD-H 10×250 mm column, 80/20 CO₂/PrOH) to give the title compound (10.9 mg). By analytical chiral SFC (Chiralpak AD-H 4.6×250 mm column, 80:20 CO₂/PrOH, 2.5 mL/min.), this product was observed to have t_{R}=2.93 min. [α]p=−58° (MeCN, c=0.73).

### Step D: (+)-Ethyl 1-(hydroxymethyl)-3-oxoisoindoline-1-carboxylate

(±)-Ethyl 1-(hydroxymethyl)-3-oxoisoindoline-1-carboxylate (32.1 mg, 0.136 mmol) was purified by chiral SFC chromatography (Chiralpak AD-H 10×250 mm column, 80/20 CO₂/PrOH) to give the title compound (11.2 mg). By analytical chiral SFC (Chiralpak AD-H 4.6×250 mm column, 80:20 CO₂/PrOH, 2.5 mL/min.), this product was observed to have t_{R}=4.10 min. [α]_{D}=+62° (MeCN, c=0.56).

### Preparation 7: (+)- and (−)-Benzyl 1-(hydroxymethyl)-2-methyl-3-oxoisoindoline-1-carboxylate

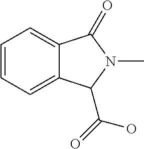

### Step A: Lithium (±)-2-Methyl-3-oxoisoindoline-1-carboxylic acid

To a solution of (±)-ethyl 2-methyl-3-oxoisoindoline-1-carboxylate (605 mg, 2.76 mmol) in THF (9.0 mL) was added MeOH (3.0 mL) and 1.0 M aq. LiOH (3.0 mL, 3.0 mmol). The resulting bright yellow solution was stirred at room temperature for 1 hours. After diluting the reaction mixture with MeCN, its volatile components were removed by rotary evaporation. The resulting pale yellow oil was dissolved in a mixture of MeOH and MeCN, and a large volume of toluene was added. Again, the volatile components of the solution were removed by rotary evaporation, and the residue was dried under high vacuum to afford the title compound as a white solid (623 mg, 75% yield). ¹H NMR (400 MHz, CDCl₃) δ 3.18 (s, 3H), 4.99 (s, 1H), 7.43-7.48 (m, 1H), 7.55 (td, J=7.5, 1.3 Hz, 1H), 7.69-7.75 (m, 2H).

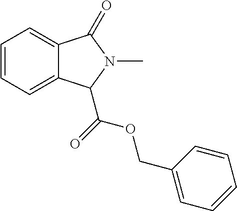 [SMILES: CN1C(=O)c2ccccc2C1C(=O)OCc1ccccc1]

### Step B: Lithium (±)-Benzyl 2-Methyl-3-oxoisoindoline-1-carboxylate

(±)-2-Methyl-3-oxoisoindoline-1-carboxylic acid (624 mg, 3.15 mmol) was dissolved in dimethylacetamide (6.0 mL) with gentle heating. The solution was cooled to room temperature and benzyl bromide (0.50 mL, 4.2 mmol) was added. The resulting yellow solution was heated at 60° C. for 1.25 hours, and then concentrated. The residue was partitioned between MTBE and water. The organic layer was isolated, washed two more times with water, and concentrated by rotary evaporation. The resulting white, crystalline solid was purified by repeated MPLC (gradient from ca. 2:3 to ca. 4:1 EtOAc/heptane) to afford the title compound (470. mg, 53% yield) as a white solid. LCMS (ESI) m/z: 282.2 [M+H] (100%). ¹H NMR (400 MHz, CDCl₃) δ 3.19 (s, 3H), 5.11 (s, 1H), 5.19 (d, J=12.1 Hz, 1H), 5.29 (d, J=12.1 Hz, 1H), 7.31-7.42 (m, 5H), 7.48-7.57 (m, 3H), 7.80-7.90 (m, 1H).

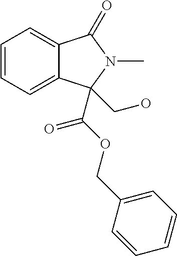 [SMILES: CN1C(=O)c2ccccc2C1(CO)C(=O)OCc1ccccc1]

### Step C: (±)-Benzyl 1-(Hydroxymethyl)-2-methyl-3-oxoisoindoline-1-carboxylate

To a solution of (±)-benzyl 2-methyl-3-oxoisoindoline-1-carboxylate (467 mg, 1.66 mmol) in 1,4-dioxane (3.3 mL) was added paraformaldehyde (108 mg, 3.60 mmol). To the white suspension was then added DBU (50 μL, 0.33 mmol). The reaction mixture turned instantly bright yellow and then faded to white. The reaction mixture was stirred at room temperature for 1 hours, and then the solvent was removed by rotary evaporation. The residue was purified by MPLC (gradient from 2:3 to 4:1 EtOAc/heptane) to afford the title compound (471 mg, 91% yield) as a clear, colorless oil. LCMS (ESI) m/z: 312.1 [M+H] (100%). ¹H NMR (400 MHz, CDCl₃) δ 2.05 (t, J=6.9 Hz, 1H), 3.16 (s, 3H), 4.15 (dd, J=12.0, 6.9 Hz, 1H), 4.31 (dd, J=11.9, 6.8 Hz, 1H), 5.11 (d, J=12.3 Hz, 1H), 5.24 (d, J=12.2 Hz, 1H), 7.18-7.24 (m, 2H), 7.30-7.34 (m, 3H), 7.50-7.56 (m, 3H), 7.83-7.88 (m, 1H).

### Preparation 8: Ethyl 7-(Hydroxymethyl)-6,7-dihydro-5H-cyclopenta[b]pyridine-7-carboxylate

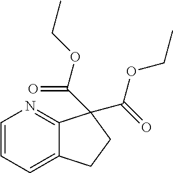 [SMILES: CCOC(=O)C1(C(=O)OCC)CCc2cccnc21]

### Step A: Diethyl 5,6-Dihydro-7H-cyclopenta[b]pyridine-7,7-dicarboxylate

To a solution of 6,7-dihydro-5H-cyclopenta[b]pyridine (1.49 mL, 12.7 mmol) in 2-MeTHF (32 mL), cooled in a dry ice/acetone bath, was added TMEDA (2.87 mL, 19.1 mmol) and tert-butyllithium (11.3 mL, 19.1 mmol, 1.7 M in pentane), affording a dark brown solution. This solution was transferred to a separate flask, which contained a solution of ethyl cyanoformate (3.92 mL, 39.5 mmol) in 2-MeTHF (32 mL), also cooled in a dry ice/acetone bath. The reaction mixture was allowed to warm to room temperature, slowly. After 20 min., the reaction was quenched with water and then extracted with EtOAc. Brine was added to mitigate the formation of an emulsion. The organic layer was isolated, washed with 1.0 M aq. HCl and brine, dried over Na₂SO₄, and concentrated to dryness. The residue was purified by MPLC (gradient from pure heptane to 7:3 EtOAc/heptane) to give the title compound (2.49 g, 74% yield). LCMS (ESI) m/z: 264.3 [M+H] (75%). ¹H NMR (400 MHz, CDCl₃) δ 1.27 (t, J=7.1 Hz, 6H), 2.78-2.84 (m, 2H), 2.98-3.04 (m, 2H), 4.20-4.33 (m, 4H), 7.16 (dd, J=7.7, 4.8 Hz, 1H), 7.53-7.58 (m, 1H), 8.51 (m, J=4.9, 1.6, 0.9, 0.9 Hz, 1H).

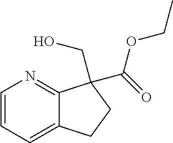 [SMILES: CCOC(=O)C1(CO)CCc2cccnc21]

### Step B: (±)-Ethyl 7-(Hydroxymethyl)-6,7-dihydro-5H-cyclopenta[b]pyridine-7-carboxylate

To solution of diethyl 5,6-dihydro-7H-cyclopenta[b]pyridine-7,7-dicarboxylate (1.41 g, 5.35 mmol) in THF (15 mL) was added LiAl(OtBu)₃H (11 mL, 11 mmol, 1.0 M in THF), dropwise at room temperature. The reaction mixture was heated to reflux for 30 min., after which it was quenched with 10% (w/v) aq. KHSO₄ (20 mL) and extracted with CH₂Cl₂. The aqueous layer was isolated and extracted again with CH₂Cl₂. The combined CH₂Cl₂ layers were washed with brine, dried over Na₂SO₄, filtered, and concentrated to dryness. The residue was purified by MPLC (gradient from pure heptane to 1:1 2-propanol/heptane) to give the title compound (547 mg, 46% yield). LCMS (ESI) m/z: 222.3 [M+H] (100%). ¹H NMR (400 MHz, CDCl₃) δ 1.22 (t, J=7.1 Hz, 3H), 2.24 (dt, J=13.4, 8.4 Hz, 1H), 2.52 (ddd, J=13.2, 8.8, 3.9 Hz, 1H), 2.95 (ddd, J=16.4, 8.8, 3.9 Hz, 1H), 3.01-3.12 (m, 1H), 3.86 (br. s., 1H), 3.99-4.09 (m, 2H), 4.18 (dq, J=10.7, 7.1 Hz, 1H), 4.22 (dq, J=10.8, 7.1 Hz, 1H), 7.15 (dd, J=7.6, 5.1 Hz, 1H), 7.58 (d, J=7.4 Hz, 1H), 8.39 (d, J=4.7 Hz, 1H).

### Step C: (−)-Ethyl 7-(Hydroxymethyl)-6,7-dihydro-5H-cyclopenta[b]pyridine-7-carboxylate

(±)-Ethyl 7-(hydroxymethyl)-6,7-dihydro-5H-cyclopenta[b]pyridine-7-carboxylate (16 g, 72 mmol) was purified by chiral SFC (Chiralpak AD-H, 20×250 mm, 4:1 CO₂/EtOH) to give the title compound (7.11 g). By analytical chiral SFC (Chiralpak AD-H 4.6×250 mm column, 4:1 CO₂/EtOH, 2.5 mL/min.), this product was observed to have t_{R}=2.29 min. [α]_{D}=−37° (MeOH, c=3.5).

### Step D: (+)-Ethyl 7-(Hydroxymethyl)-6,7-dihydro-5H-cyclopenta[b]pyridine-7-carboxylate

(±)-Ethyl 7-(hydroxymethyl)-6,7-dihydro-5H-cyclopenta[b]pyridine-7-carboxylate (16 g, 72 mmol) was purified by chiral SFC (Chiralpak AD-H 20×250 mm column, 4:1 CO₂/EtOH) to give the title compound (7.19 g). By analytical chiral SFC (Chiralpak AD-H 4.6×250 mm column, 4:1 CO₂/EtOH, 2.5 mL/min.), this product was observed to have t_{R}=5.12 min. [α]_{D}=+36° (MeOH, c=2.2).

### Preparation 9: Ethyl 8-(Hydroxymethyl)-5,6,7,8-tetrahydroquinoline-8-carboxylate

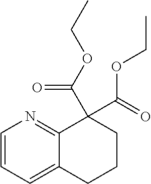 [SMILES: CCOC(=O)C1(C(=O)OCC)CCCc2cccnc21]

Step A: Diethyl 6,7-Dihydroquinoline-8,8(5H)-dicarboxylate

To a solution of 5,6,7,8-tetrahydro-quinoline (370 mg, 2.78 mmol) in 2-MeTHF (6 mL), cooled in a dry ice/acetone bath, was added TMEDA (0.62 mL, 4.17 mmol) and tert-butyllithium (2.45 mL, 4.17 mmol, 1.7 M in pentane), affording a dark brown solution. This solution was transferred to a separate flask, which contained a solution of ethyl cyanoformate (0.84 mL, 8.61 mmol) in 2-MeTHF (6 mL), also cooled in a dry ice/acetone bath. The reaction mixture was allowed to warm to room temperature, slowly. After 20 min., the reaction was quenched with water and then extracted with EtOAc. Brine was added to mitigate the formation of an emulsion. The organic layer was isolated, washed with 1.0 M aq. HCl and brine, dried over Na₂SO₄, and concentrated to dryness. The residue was purified by MPLC (gradient from pure heptane to 7:3 EtOAc/heptane) to give the title compound (555 mg, 72% yield). LCMS (ESI) m/z: 278.2 [M+H] (100%). ¹H NMR (400 MHz, CDCl₃) δ 1.26 (t, J=7.1 Hz, 6H), 1.77-1.86 (m, 2H), 2.51-2.57 (m, 2H), 2.84 (t, J=6.64 Hz, 2H), 4.22-4.30 (m, 4H), 7.12-7.15 (m, 1H), 7.40-7.43 (m, 1H), 8.43-8.47 (m, 1H).

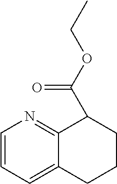

### Step B: (±)-Ethyl 5,6,7,8-Tetrahydroquinoline-8-carboxylate

To solution of diethyl 6,7-dihydroquinoline-8,8(5H)-dicarboxylate (137 mg, 0.494 mmol) in EtOH (0.55 mL) was added 1.0 M aq. NaOH (0.50 mL, 0.50 mmol). The reaction mixture was stirred at room temperature for 12 hours, and then the EtOH was removed by rotary evaporation. The residue was added to water and then extracted twice with EtOAc. The combined organic layers were dried over Na₂SO₄, filtered, and concentrated to dryness. The resulting crude reaction mixture was purified by MPLC (gradient from pure heptane to 1:1 2-propanol/heptane) to give the title compound (93 mg, 52 wt. % pure, as a mixture with diethyl 6,7-dihydroquinoline-8,8(5H)-dicarboxylate). LCMS (ESI) m/z: [M+H] (100%). This material was used without further purification. ¹H NMR (400 MHz, CDCl₃) δ 1.28 (t, J=7.1 Hz, 3H), 1.80-1.87 (m, 1H), 1.93-2.04 (m, 1H), 2.13-2.24 (m, 2H), 2.72-2.81 (m, 2H), 3.97 (t, J=6.5 Hz, 1H), 4.18-4.24 (m, 2H), 7.12-7.15 (m, 1H), 7.38-7.44 (m, 1H), 8.40-8.42 (m, 1H).

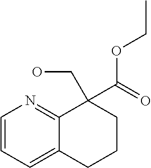 [SMILES: CCOC(=O)C1(CO)CCCc2cccnc21]

### Step C: (±)-Ethyl 8-(Hydroxymethyl)-5,6,7,8-tetrahydroquinoline-8-carboxylate

To solution of (±)-ethyl 5,6,7,8-tetrahydroquinoline-8-carboxylate (93 mg, 0.45 mmol) in 1,4-dioxane (0.8 mL) was added aq. formaldehyde (71 μL, 0.95 mmol, ca. 37 wt.) and DBU (14 μL, 0.091 mmol). The reaction mixture was heated to 100° C. by microwave irradiation for 45 min, after which it was concentrated to dryness and partitioned between EtOAc and H₂O. The layers were separated and the aqueous layer was washed twice with EtOAc. The combined organic layers were washed with brine, dried over Na₂SO₄ and concentrated to dryness. The resulting crude reaction mixture was purified by flash chromatography (gradient from pure heptane to 1:1 2-propanol/heptane) to afford the title compound (88 mg, 56 wt. % pure) as a mixture with diethyl 6,7-dihydroquinoline-8,8(5H)-dicarboxylate. This product was used without further purification. LCMS (ESI) m/z: 236.3 [M+H] (100%). ¹H NMR (400 MHz, CDCl₃) δ 1.21 (t, J=7.0 Hz, 3H), 1.67-1.78 (m, 3H), 1.87-1.95 (m, 1H), 2.22-2.29 (m, 1H), 2.71-2.84 (m, 2H), 3.91-4.06 (m, 2H),) 4.20 (q, J=7.0 Hz, 2H), 7.15-7.18 (m, 1H), 7.45-7.47 (m, 1H), 8.35-8.38 (m, 1H).

### Preparation 10: (±)-Ethyl 1-(Hydroxymethyl)-3-oxo-1,3-dihydro-2-benzofuran-1-carboxylate

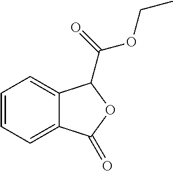

### Step A: Ethyl 3-Oxo-1,3-dihydro-2-benzofuran-1-carboxylate

To (±)-ethyl 2-(1-bromo-2-ethoxy-2-oxoethyl)benzoate (514 mg, 1.63 mmol) in DMAc (3.0 mL) was added KOAc (700. mg, 7.13 mmol). The resulting mixture was heated in an aluminum block at 85° C. for 16 hours. The reaction mixture was partitioned between MTBE and water. The organic layer was washed 2 times with water. The solvent was removed from the organic layer to afford the intermediate acetate as a yellow liquid. This liquid was dissolved in EtOH (16 mL) and thionyl chloride (0.15 mL, 2.1 mmol) was added. The resulting solution was heated to 70° C. for 2.5 hours and then cooled to 40° C. and stirred for 16 hours. The solvent was removed from the reaction mixture, and the residue was purified by MPLC (gradient from 1:9 EtOAc/heptane to 1:1 EtOAc/heptane) to give the title compound (274 mg, 81%) as a clear oil. LCMS (ESI) m/z: 207.0 [M+H] (100%). ¹H NMR (400 MHz, CDCl₃) δ 1.32 (t, J=7.2 Hz, 3H), 4.27 (dq, J=10.8, 7.1 Hz, 1H), 4.33 (dq, J=10.8, 7.1 Hz, 1H), 5.89 (s, 1H), 7.61 (t, J=7.4 Hz, 1H), 7.67-7.71 (m, 1H), 7.71-7.76 (m, 1H), 7.95 (d, J=7.6 Hz, 1H).

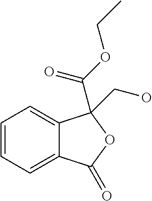 [SMILES: CCOC(=O)C1(CO)OC(=O)c2ccccc21]

### Step B: (±)-Ethyl 1-(Hydroxymethyl)-3-oxo-1,3-dihydro-2-benzofuran-1-carboxylate

To a solution of (±)-ethyl 3-Oxo-1,3-dihydro-2-benzofuran-1-carboxylate (265 mg, 1.28 mmol) in 1,4-dioxane (1.3 mL) was added paraformaldehyde (121 mg, 4.03 mmol) followed by DBU (38.5 μL, 0.257 mmol). The resulting mixture was stirred at room temperature for 1.5 hours. The solvent was then removed by rotary evaporation, and the residue was purified by MPLC (gradient from 1:3 EtOAc/heptane to 3:1 EtOAc/heptane) to give the title compound (223 mg, 73%) as a white solid. LCMS (ESI) m/z: 191.0 [M-OEt] (76%), 237.0 [M+H] (100%), 259.0 [M+Na] (83%). ¹H NMR (400 MHz, CDCl₃) δ 1.28 (t, J=7.1 Hz, 3H), 2.30 (dd, J=8.8, 6.1 Hz, 1H), 4.04 (dd, J=12.2, 5.9 Hz, 1H), 4.24 (dq, J=10.7, 7.1 Hz, 1H), 4.31 (dq, J=10.7, 7.1 Hz, 1H), 4.33 (dd, J=12.2, 8.8 Hz, 1H), 7.63 (td, J=7.5, 0.7 Hz, 1H), 7.68-7.71 (m, 1H), 7.72-7.76 (m, 1H), 7.94 (d, J=7.6 Hz, 1H).

### Step C: (+)-Ethyl 1-(Hydroxymethyl)-3-oxo-1,3-dihydro-2-benzofuran-1-carboxylate

(±)-Ethyl 1-(hydroxymethyl)-3-oxo-1,3-dihydro-2-benzofuran-1-carboxylate (222 mg, 0.940 mmol) was purified by chiral SFC (Chiralpak AD-H 10×250 mm column, 17:3 CO₂/EtOH) to give the title compound (92.4 mg). By analytical chiral SFC (Chiralpak AD-H 4.6×250 mm column, 17:3 CO₂/EtOH, 2.5 mL/min.), this product was observed to have t_{R}=2.93 min. [α]_{D}=+61° (MeCN, c=0.77). LCMS (ESI) m/z: 237.0 [M+H] (100%), 259.0 [M+Na] (90. %).

### Step D: (−)-Ethyl 1-(Hydroxymethyl)-3-oxo-1,3-dihydro-2-benzofuran-1-carboxylate

(±)-Ethyl 1-(hydroxymethyl)-3-oxo-1,3-dihydro-2-benzofuran-1-carboxylate (222 mg, 0.940 mmol) was purified by chiral SFC (Chiralpak AD-H, 10×250 mm, 17:3 CO₂/EtOH) to give the title compound (88.1 mg). By analytical chiral SFC (Chiralpak AD-H 4.6×250 mm column, 17:3 CO₂/EtOH, 2.5 mL/min.), this product was observed to have t_{R}=2.55 min. [α]_{D}=−60. ° (MeCN, c=0.73). LCMS (ESI) m/z: 237.0 [M+H] (95%), 259.0 [M+Na] (100%).

### Preparation 11: (±)-Ethyl 1-(Hydroxymethyl)indane-1-carboxylate

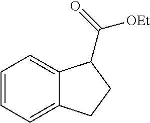 [SMILES: CCOC(=O)C1CCc2ccccc21]

### Step A: (±)-Ethyl Indane-1-carboxylate

To a stirred solution of α-tetralone (10.0 g, 68 mmol) in EtOH (50 mL) was added BF₃.Et₂O (30 mL, 240 mmol). After 20 min., this solution was added to a suspension of Pb(OAc)₄ (31.8 g, 68 mmol) in toluene (200 mL). The reaction was stirred for 3 days. The reaction was quenched with cold water (300 mL) and stirred. The aqueous layer was isolated and extracted with EtOAc. The combined organic layers were washed with aq. NaHCO₃ (2×) and brine, dried over Na₂SO₄, filtered, and concentrated. The residue was purified MPLC (gradient from pure heptane to 1:1 EtOAc/heptane) to afford the title compound (4.56 g, 35%) as an orange oil. ¹H NMR (400 MHz, CDCl₃) δ 1.30 (t, J=7.1 Hz, 3H), 2.34 (dtd, J=12.9, 8.6, 8.6, 5.7 Hz, 1H), 2.46 (ddt, J=13.1, 8.6, 6.4, 6.4 Hz, 1H), 2.93 (ddd, J=15.8, 8.7, 6.5 Hz, 1H), 3.12 (ddd, J=15.8, 8.6, 5.9 Hz, 1H), 4.05 (dd, J=8.6, 6.4 Hz, 1H), 4.19 (dq, J=10.7, 7.2 Hz, 1H), 4.22 (dq, J=10.7, 7.2 Hz, 1H), 7.16-7.28 (m, 3H), 7.37-7.42 (m, 1H).

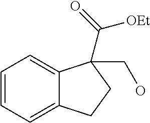 [SMILES: CCOC(=O)C1(CO)CCc2ccccc21]

### Step B: (±)-Ethyl 1-(Hydroxymethyl)indane-1-carboxylate

To an oven dried 250 mL round bottom flask containing a solution of diisopropylamine (6.0 mL, 43 mmol) in THF (60 mL) and cooled in a dry ice/acetone bath was added n-butyllithium (14.5 mL, 36 mmol, 2.5 M in hexane). After stirring for 30 minutes, a solution of (±)-ethyl indane-1-carboxylate (4.5 g, 23.6 mmol) in THF (40 mL) was added, dropwise over 15 minutes via a syringe, affording a yellow solution. The reaction mixture was stirred for 1 hour, then paraformaldehyde (1.9 g, 64 mmol) was added in one portion. After changing the cold bath to ice/water, the reaction mixture was allowed to stir for 30 min. The reaction was quenched with 1 M aq. NH₄Cl and extracted with EtOAc. The organic layer was isolated, washed with brine, dried over Na₂SO₄, and concentrated to a yellow oil. This oil was purified by MPLC (gradient from 1:9 EtOAc/heptane to 1:1 EtOAc/heptane) to afford the title compound (3.92 g, 80%) as a clear, light green oil. ¹H NMR (400 MHz, CDCl₃) δ 1.22 (t, J=7.1 Hz, 3H), 2.29 (ddd, J=13.4, 8.4, 5.3 Hz, 1H), 2.48 (dd, J=7.1, 6.5 Hz, 1H), 2.60 (ddd, J=13.3, 8.8, 7.0 Hz, 1H), 2.94-3.12 (m, 2H), 3.66 (dd, J=11.2, 7.2 Hz, 1H), 3.99 (dd, J=11.1, 6.2 Hz, 1H), 4.11-4.24 (m, 2H), 7.16-7.29 (m, 4H).

### Preparation 12: (±)-Ethyl 1-(Hydroxymethyl)-1,2,3,4-tetrahydronaphthalene-1-carboxylate

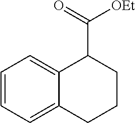 [SMILES: CCOC(=O)C1CCCc2ccccc21]

### Step A: (±)-Ethyl 1,2,3,4-Tetrahydronaphthalene-1-carboxylate

A solution of 1-benzosuberone (3.3 g, 21 mmol) and BF₃.Et₂O (15 mL, 120 mmol) in EtOH (20 ml, anhydrous) was added to a suspension of Pb(OAc)₄ (9.3 g, 20 mmol) in benzene (100. mL). The reaction mixture, which turned yellow, was stirred for 22 hours at room temperature. Subsequently, the reaction was quenched with cold water (250 mL). The aq. layer was isolated and extracted with EtOAc (2×60 mL). The combined organic layers were washed sequentially with sat. aq. NaHCO₃ and water, dried over MgSO₄, and concentrated to dryness by rotary evaporation. The residue was purified by repeated MPLC (gradient from pure heptane to ca. 9:11 EtOAc/heptane) to afford the title compound (200 mg, 4.9% yield) as an impure oil, which was used without further purification.

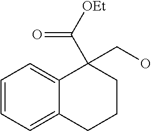 [SMILES: CCOC(=O)C1(CO)CCCc2ccccc21]

### Step B: (±)-Ethyl 1-(Hydroxymethyl)-1,2,3,4-tetrahydronaphthalene-1-carboxylate

A solution of diisopropylamine (0.25 mL, 1.8 mmol) in THF (4.0 mL), contained in an oven-dried flask under a nitrogen atmosphere, was cooled in a dry ice/acetone bath. n-Butyllithium (0.60 mL, 1.5 mmol, 2.5 M in hexane) was added, and the resulting solution was stirred for 30 min. A solution of (±)-ethyl 1,2,3,4-tetrahydronaphthalene-1-carboxylate (200 mg, 1.0 mmol) in THF (2.0 mL) was then added dropwise via a syringe over a period of 5 min., affording a yellow solution. After stirring for 60 min., paraformaldehyde (80. mg, 2.3 mmol) was added in one portion. The cold bath was changed to ice/water, and stirring was continued for 30 min. The solution was then quenched with 1 M citric acid (20 mL) and extracted with EtOAc (20 mL). The organic layer was isolated, washed with brine (10 mL), dried over Na₂SO₄, and concentrated to a yellow oil. This oil was adsorbed onto silica gel and purified by MPLC (gradient from pure heptane to 2:3 EtOAc/heptane) to afford the title compound (175 mg, 76% yield) as a clear oil. ¹H NMR (400 MHz, CDCl₃) δ 1.21 (t, J=7.1 Hz, 3H), 1.78-1.99 (m, 2H), 2.15 (ddd, J=13.4, 10.9, 3.3 Hz, 1H), 2.33 (ddd, J=13.3, 6.4, 2.9 Hz, 1H), 2.71-2.92 (m, 3H), 3.60 (dd, J=11.3, 8.4 Hz, 1H), 4.08 (d, J=11.7 Hz, 1H), 4.15 (dq, J=10.9, 7.1 Hz, 1H), 4.21 (dq, J=10.9, 7.1 Hz, 1H), 7.06-7.21 (m, 4H).

### Preparation 13: [3-(Dimethylcarbamoyl)-4-nitrophenyl]acetic Acid

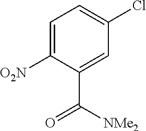 [SMILES: CN(C)C(=O)c1cc(Cl)ccc1[N+](=O)[O-]]

### Step A: 5-Chloro-N,N-dimethyl-2-nitrobenzamide

To a solution of 5-chloro-2-nitrobenzoic acid (100 g, 0.5 mol) in THF (800 mL) was added, portion-wise, 1,1′-carbonyldiimidazole (81 g, 0.5 mol). The reaction mixture was refluxed for 1 hour and then cooled to room temperature. Triethylamine (69.2 mL, 0.5 mol) and dimethylamine hydrochloride (38.4 g, 0.471 mol) were added, and the reaction mixture was stirred at room temperature overnight. The reaction mixture was again refluxed for 6 hours and then concentrated to dryness. The residue was dissolved with EtOAc, washed sequentially with sat. aq. NaHCO₃ (3×500 mL), sat. aq. NH₄Cl (4×500 mL), and brine (2×500 mL), dried over MgSO₄, and concentrated to dryness to afford the title compound (97 g, 86%) as a yellow solid. ¹H NMR (400 MHz, CDCl₃) δ 2.85 (s, 3H), 3.15 (s, 3H), 7.36 (d, J=2.3 Hz, 1H), 7.52 (dd, J=2.2, 8.7 Hz, 1H), 8.13 (d, J=8.7 Hz, 1H).

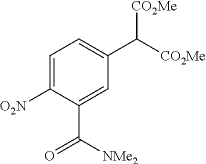 [SMILES: COC(=O)C(C(=O)OC)c1ccc([N+](=O)[O-])c(C(=O)N(C)C)c1]

### Step B: Dimethyl [3-(Dimethylcarbamoyl)-4-nitrophenyl]malonate

To a stirred suspension of sodium hydride (28 g, 0.70 mol, 60% dispersion in mineral oil) in DMF (1 L) at 0° C. was added dimethylmalonate (80. mL, 0.70 mol), dropwise. The reaction mixture was returned to room temperature while stirring, after which 5-chloro-N,N-dimethyl-2-nitrobenzamide (80. g, 0.35 mol) was added at once. The reaction mixture was then heated at 120° C. for 3 hours. The solvent was subsequently removed, and the residue was diluted with EtOAc (1 L) and washed with sat. aq. NH₄Cl (5×300 mL) and brine (3×500 mL). The organic layer was finally concentrated to dryness to afford title compound as brown oil (124 g). This material was used in the following step without further purification. ¹H NMR (400 MHz, CDCl₃) δ 2.85 (s, 3H), 3.14 (s, 3H), 3.73 (s, 6H), 4.72 (s, 1H), 7.44 (s, 1H), 7.6 (d, J=8.7 Hz, 1H), 8.16 (d, J=8.7 Hz, 1H).

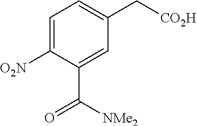 [SMILES: CN(C)C(=O)c1cc(CC(=O)O)ccc1[N+](=O)[O-]]

### Step C: [3-(Dimethylcarbamoyl)-4-nitrophenyl]acetic Acid

Dimethyl [3-(dimethylcarbamoyl)-4-nitrophenyl]malonate (50.2 g, 155 mmol), conc. aq. HCl (150 mL), and water (5 mL) were refluxed for 3 hours. The reaction mixture was cooled, and water (300 mL) was added. The mixture was basified with solid NaHCO₃, and subsequently washed with EtOAc (3×300 mL). The basic aqueous layer was then acidified with conc. aq. HCl and extracted with EtOAc (5×300 mL). The combined organic layers were dried over MgSO₄ and concentrated to dryness to afford a dark oil (37.1 g). This oil was purified by crystallization from EtOAc, and the isolated crystals were dried in a vacuum oven at 60° C. to afford the title compound (17.9 g, 50% yield over 2 steps) as a red solid. ¹H NMR (400 MHz, CDCl₃) δ 2.78 (s, 3H), 3.16 (s, 3H), 3.61 (s, 2H), 7.27 (s, 1H), 7.42 (d, J=8.7 Hz, 1H), 8.12 (d, J=8.7 Hz, 1H).

### Preparation 14: Diethyl 2-(4-Amino-3-(dimethylcarbamoyl)phenyl)malonate

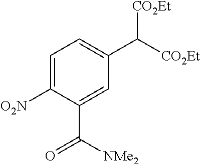 [SMILES: CCOC(=O)C(C(=O)OCC)c1ccc([N+](=O)[O-])c(C(=O)N(C)C)c1]

### Step A: Diethyl 2-(3-(dimethylcarbamoyl)-4-nitrophenyl)malonate

The title compound was prepared following the general route of Preparation 13 using diethylmalonate. ¹H NMR (400 MHz, CDCl₃) δ 1.28 (t, J=7.1 Hz, 5H), 2.87 (s, 3H), 3.17 (s, 3H), 4.18-4.29 (m, 4H), 4.70 (s, 1H), 7.47 (d, J=2.0 Hz, 1H), 7.61-7.66 (m, 1H), 8.19 (d, J=8.6 Hz, 1H).

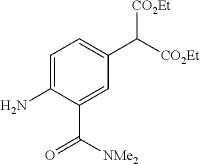 [SMILES: CCOC(=O)C(C(=O)OCC)c1ccc(N)c(C(=O)N(C)C)c1]

### Step B: Diethyl 2-(4-Amino-3-(dimethylcarbamoyl)phenyl)malonate

To a rapidly stirred slurry of diethyl 2-(3-(dimethylcarbamoyl)-4-nitrophenyl)malonate (13.0 g, 36.9 mmoles) in EtOH (130. mL) was added iron filings (6.18 g, 111 mmoles) followed by AcOH (21.1 mL, 369 mmoles). The reaction was warmed to reflux. Ca. 15 min. after reaching reflux the previously clear pale yellow solution became a white slurry). The reaction mixture was then cooled to room temperature, diluted with water (250 mL), and filtered to remove residual iron filings. The solutions were extracted with DCM (2×200 mL). The combined DCM layers were dried over MgSO₄ and concentrated under vacuum to afford the title compound as an oil (15 g, 126% of theoretical). ¹H NMR (400 MHz, CDCl₃) δ 1.25 (t, J=7.1 Hz, 6H), 4.17-4.23 (m, 4H), 4.47 (s, 1H), 6.69 (d, J=8.3 Hz, 1H), 7.16 (dd, J=8.3, 2.2 Hz, 1H), 7.21 (d, J=2.1 Hz, 1H).

### Preparation 15: [3-(Dimethylcarbamoyl)-4-nitrophenyl]acetic Acid

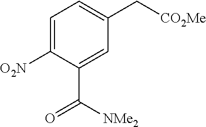 [SMILES: COC(=O)Cc1ccc([N+](=O)[O-])c(C(=O)N(C)C)c1]

### Step A: Methyl [3-(Dimethylcarbamoyl)-4-nitrophenyl]acetate

To a solution [3-(dimethylcarbamoyl)-4-nitrophenyl]acetic acid (17.9 g, 71 mmol) in MeOH (120 mL) at 0° C. was added acetyl chloride (6.1 mL, 85.1 mmol). The reaction mixture was then returned to room temperature, stirred for 3 hours, and then concentrated to dryness. The residue was dissolved in EtOAc and washed sequentially with sat. aq. NaHCO₃ (5×100 mL) and brine (3×200 mL). The organic layer was dried over MgSO₄ and concentrated to afford a brown oil (20.5 g). This oil was purified by MPLC (gradient from 1:9 to 2:3 EtOAc/heptane) to afford the title compound (16.6 g, 87% yield) as an orange solid. ¹H NMR (400 MHz, CDCl₃) δ 2.83 (s, 3H), 3.14 (s, 3H), 3.70 (s, 3H), 3.71 (s, 2H), 7.30 (d, J=1.8 Hz, 1H), 7.45 (dd, J=1.8, 8.7 Hz, 1H), 8.14 (d, J=8.2 Hz, 1H).

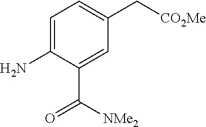 [SMILES: COC(=O)Cc1ccc(N)c(C(=O)N(C)C)c1]

### Step B: Methyl [4-Amino-3-(dimethylcarbamoyl)phenyl]acetate

To a stirred solution of methyl [3-(dimethylcarbamoyl)-4-nitrophenyl]acetate (25.0 g, 93.9 mmol) in THF (500 mL) was added 5% Pd/C (5 g, 2.3 mmol) as a slurry in a minimal amount of toluene (ca. 10 mL). The reaction mixture was shaken under an H₂ atmosphere (50 bar) overnight at room temperature. The reaction mixture was then filtered through Celite, and the filtrate was concentrated to dryness to afford the title compound (22.1 g, 100%) as a white solid. ¹H NMR (300 MHz, CDCl₃) δ 3.05 (br. s., 6H), 3.49 (s, 2H), 3.67 (s, 3H), 4.31 (br. s., 2H), 6.66 (d, J=8.2 Hz, 1H), 7.02-7.08 (m, 2H).

### Preparation 16: Methyl (4-Amino-3-methylphenyl)acetate

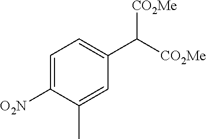 [SMILES: COC(=O)C(C(=O)OC)c1ccc([N+](=O)[O-])c(C)c1]

### Step A: Dimethyl (3-Methyl-4-nitrophenyl)malonate

A mixture of 4-fluoro-2-methyl-1-nitrobenzene (10 g, 64 mmol), cesium carbonate (41.7 g, 128 mmol) and dimethylmalonate (8.13 mL, 96 mmol) in MeCN (100 mL) was refluxed for 5 hours with vigorous stirring. Additional cesium carbonate (10.4 g, 32 mmol) was added, and refluxing was continued for 72 hours more. The reaction mixture was then cooled and concentrated to dryness. The residue was partitioned between EtOAc (400 mL) and sat. aq. NH₄Cl (400 mL). The organic layer was isolated, dried over MgSO₄, and concentrated to afford an oil. The oil was taken up in a 1:1 (v/v) mixture of EtOAc and heptane, and the title compound (11.1 g, 65% yield) was collected by filtration as a tan solid. ¹H NMR (400 MHz, CDCl₃) δ 2.60 (s, 3H), 3.78 (s, 6H), 4.68 (s, 1H), 7.39 (m, 2H), 7.96 (d, 1H).

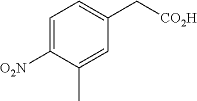

### Step B: (3-Methyl-4-nitrophenyl)acetic Acid

A mixture of dimethyl (3-methyl-4-nitrophenyl)malonate (11.1 g, 41.6 mmol) and 6.0 M aq. HCl was refluxed for 3.5 hours. The reaction mixture was then cooled to room temperature and extracted with CH₂Cl₂ (200 mL). The organic layer was isolated and extracted with sat. aq. NaHCO₃ (2×150 mL). The basic aqueous layer was washed with CH₂Cl₂, acidified, and then extracted again with CH₂Cl₂ (2×150 mL). The organic layers were combined, dried over MgSO₄, and concentrated to dryness to afford the title compound (3.97 g, 49% yield) as a solid. ¹H NMR (400 MHz, CDCl₃) δ 2.60 (s, 3H), 3.71 (s, 2H), 7.25 (m, 2H), 7.96 (d, 1H), 10.42 (br s, 1H).

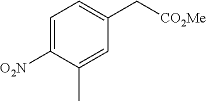

### Step C: Methyl (3-Methyl-4-nitrophenyl)acetate

Acetyl chloride (2.3 mL, 34 mmol) was added dropwise to MeOH (55 mL) at 0° C. (3-Methyl-4-nitrophenyl)acetic acid (5.42 g, 27.8 mmol) was added, and the reaction mixture was stirred at room temperature for 18 hours. Subsequently, the reaction mixture was concentrated to an oil and re-dissolved in EtOAc (100 mL). This solution was washed with sat. aq. NaHCO₃ (100 mL), dried over MgSO₄, and concentrated to afford the title compound (5.42 g, 93% yield) as an oil. ¹H NMR (400 MHz, CDCl₃) δ 2.59 (s, 3H), 3.66 (s, 2H), 3.70 (s, 3H), 7.24 (m, 2H), 7.95 (d, 1H).

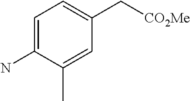

### Step D: Methyl (4-Amino-3-methylphenyl)acetate

A mixture of methyl (3-methyl-4-nitrophenyl)acetate (5.3 g, 25.3 mmol) and 5% Pd/C (1.35 g, 0.63 mmol) in THF (150 mL) was stirred under hydrogen atmosphere (50 bar) for 4 hours. The reaction mixture was then filtered through Celite, rinsing with THF. The filtrate was concentrated by rotary evaporation, and the residue was passed through a pad of silica gel, eluting with EtOAc. The solvent was removed from the filtrate to afford the title compound (4.1 g, 90% yield) as an oil. ¹H NMR (400 MHz, CDCl₃) δ 2.14 (s, 3H), 3.48 (s, 2H), 3.66 (s, 3H), 6.62 (d, 1H), 6.91-6.98 (m, 2H).

### Preparation 17: Methyl (4-Amino-3-chlorophenyl)acetate

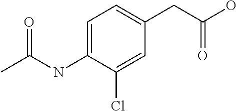

### Step A: (4-Acetamido-3-chlorophenyl)acetic Acid

To a vigorously stirred suspension of 4-aminophenylacetic acid (5.0 g, 33.1 mmol) in acetic acid (15 mL) and water (7 mL) was added acetic anhydride (3.75 mL, 39.7 mmol) dropwise at room temperature (a cold water bath was used to reduce the exotherm observed). The reaction mixture was stirred at room temperature for 1 hour. The reaction mixture was diluted with EtOH (15 mL) and water (7 mL) and a suspension of calcium hypochlorite (5.7 g, 39.7 mmol) in water (25 mL) was added portionwise at room temperature (a cold water bath was used to reduce the exotherm observed). The reaction mixture stirred at room temperature for 1 hour. The reaction mixture was poured into ice-water (200 mL) and the resulting aqueous mixture extracted with EtOAc (2×75 mL). The combined organic phases were washed with brine (2×50 mL), dried (MgSO₄), and concentrated to a small volume in vacuo. The residue was diluted with hexane and the solid collected by filtration to afford the title compound (4.93 g, crude) as a cream-colored solid. The material was used in the next step without further purification. ¹H NMR (400 MHz, DMSO-d6) δ 2.04 (s, 3H), 3.54 (s, 2H), 7.15 (dd, 1H), 7.35 (d, 1H), 7.56 (d, 1H), 9.45 (s, 1H).

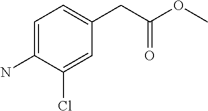

### Step B: Methyl (4-Amino-3-chlorophenyl)acetate

To a stirred solution of (4-acetamido-3-chlorophenyl)acetic acid (8.53 g, 37.5 mmol) in MeOH (85 mL) was added conc. aq. HCl (10 mL). The reaction mixture was refluxed for 1.5 hours and then returned to room temperature. The volatile components of the reaction mixture were removed, and the residue was diluted with water (100 mL) and poured into sat. aq. NaHCO₃ (250 mL). This mixture was extracted with EtOAc (2×200 mL). The combined organic layers were washed with brine (2×100 mL), dried over MgSO₄, and concentrated to afford a brown oil. This oil was purified by MPLC (eluting with 7:3 EtOAc/heptane) to give the title compound (6.9 g, 60% yield over two steps) as a light brown oil. ¹H NMR (400 MHz, DMSO-d6) δ 3.46 (s, 2H), 3.56 (s, 3H), 5.22 (br. s., 2H), 6.69 (d, 1H), 6.86 (dd, 1H), 7.05 (d, 1H).

### Example 1

### Ethyl 1R-({2-[3-(dimethylcarbamoyl)-4-({[4′-(trifluoromethyl)biphenyl-2-yl]carbonyl}amino)phenyl]acetoxy}methyl)-2-methyl-3-oxoisoindoline-1-carboxylate

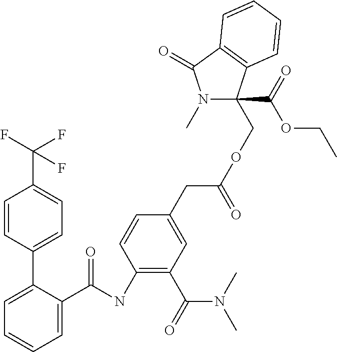 [SMILES: CCOC(=O)[C@]1(COC(=O)Cc2ccc(NC(=O)c3ccccc3-c3ccc(C(F)(F)F)cc3)c(C(=O)N(C)C)c2)c2ccccc2C(=O)N1C]

### Step A: Methyl [3-(Dimethylcarbamoyl)-4-({[4′-(trifluoromethyl)biphenyl-2-yl]carbonyl}amino)phenyl]acetate

To a suspension of 4′-(trifluoromethyl)biphenyl-2-carboxylic acid (4.10 g, 15.4 mmol) in 1,2-dichloroethane (90. mL) was added oxalyl chloride (2.0 mL, 22 mmol) followed by DMF (0.05 mL, 0.6 mmol). Gas evolution was observed and the solids dissolved over time. The reaction mixture was stirred at room temperature for 2.5 hours. The reaction mixture was then concentrated to give a yellow oil. The oil was re-dissolved in 1,2-dichloroethane (30. mL) and added dropwise to a solution of methyl [4-amino-3-(dimethylcarbamoyl)phenyl]acetate (4.50 g, 19.0 mmol) and triethylamine (7.0 mL, 50. mmol) in 1,2-dichloroethane (90. mL). The reaction mixture was stirred at room temperature temperature for 10 min. before being quenched with sat. aq. NH₄Cl (100 mL) followed by brine (100 mL). The organic layer was isolated, dried over MgSO₄, and concentrated to dryness. The residue obtained was purified by MPLC (gradient from 1:4 EtOAc/heptane to pure EtOAc) to afford the title compound (7.18 g, 96% yield) as a white solid. ¹H NMR (400 MHz, CDCl₃) δ 2.89 (br. s., 3H), 2.95 (br. s., 3H), 3.57 (d, J=2.5 Hz, 2H), 3.67 (s, 3H), 7.12 (s, 1H), 7.27-7.32 (m, 1H), 7.40 (dd, J=7.5, 1.5 Hz, 1H), 7.46-7.58 (m, J=14.4, 7.5, 1.6, 1.6, 1.4 Hz, 2H), 7.61 (s, 4H), 7.69 (d, J=7.5 Hz, 1H), 8.36 (d, J=8.0 Hz, 1H), 9.13 (br. s., 1H).

### Step B: [3-(Dimethylcarbamoyl)-4-({[4′-(trifluoromethyl)biphenyl-2-yl]carbonyl}amino)phenyl]acetic Acid

To a solution of methyl [3-(dimethylcarbamoyl)-4-({[4′-(trifluoromethyl)biphenyl-2-yl]carbonyl}amino)phenyl]acetate (7.17 g, 14.8 mmol) in THF (80. mL) and MeOH (20. mL) was added 1.0 M aq. LiOH (20. mL, 20. mmol). The reaction mixture was stirred at r.t for 16 hours and then concentrated by rotary evaporation. The residue was diluted with water (100 mL) and washed with EtOAc (100 mL). The aq. layer was acidified with conc. aq. HCl (4 mL) and afterward extracted with CH₂Cl₂ (2×150 mL). The combined organic layers were washed with brine (100 mL), dried over Na₂SO₄, and concentrated to afford the title compound (6.90 g, 99% yield) as a white solid. ¹H NMR (400 MHz, CDCl₃) δ 2.86 (br s, 3H), 2.94 (br s, 3H), 3.55 (s, 2H), 3.66 (s, 3H), 7.11 (d, J=2.4 Hz, 1H), 7.25-7.29 (m, 1H), 7.36-7.41 (m, 1H), 7.45-7.55 (m, 2H), 7.57-7.62 (m, 3H), 7.68 (dd, J=7.8, 1.4 Hz, 1H), 8.35 (d, J=8.7 Hz, 1H), 9.12 (s, 1H).

### Step C: Ethyl 1R-({2-[3-(Dimethylcarbamoyl)-4-({[4′-(trifluoromethyl)biphenyl-2-yl]carbonyl}amino)phenyl]acetoxy}methyl)-2-methyl-3-oxoisoindoline-1-carboxylate

To a solution of [3-(dimethylcarbamoyl)-4-({[4′-(trifluoromethyl)biphenyl-2-yl]carbonyl}amino)phenyl]acetic acid (2.36 g, 5.01 mmol) in CH₂Cl₂ (20. mL), was added the ethyl (1R)-1-(hydroxymethyl)-2-methyl-3-oxoisoindoline-1-carboxylate (1.04 g, 4.17 mmol), DMAP (714 mg, 5.84 mmol), and 1-ethyl-3-(3-dimethylaminopropyl)carbodiimide hydrogen chloride salt (1.12 g, 5.84 mmol). The reaction mixture was stirred at room temperature for 18 hours, after which the solvent was removed by rotary evaporation. The residue was partitioned between EtOAc and sat. aq. NH₄Cl. The organic layer was isolated, washed with sat. aq. NaHCO₃ and brine, dried over Na₂SO₄, and concentrated to dryness. The residue was purified by MPLC (gradient from pure heptane to 9:1 EtOAc/heptane) to afford the title compound (2.44 g, 83% yield). LCMS (ESI) m/z: 702.2 [M+H] (61%). ¹H NMR (400 MHz, CDCl₃) δ 1.20 (t, J=7.1 Hz, 3H), 2.84 (br. s., 3H), 2.97 (br. s., 3H), 3.09 (s, 3H), 3.32-3.43 (m, 2H), 4.12 (dq, J=10.8, 7.1 Hz, 1H), 4.24 (dq, J=10.7, 7.2 Hz, 1H), 4.68 (d, J=11.9 Hz, 1H), 4.83 (d, J=11.9 Hz, 1H), 6.91 (d, J=2.1 Hz, 1H), 7.01 (dd, J=8.6, 2.0 Hz, 1H), 7.41 (dd, J=7.6, 1.4 Hz, 1H), 7.47-7.58 (m, 5H), 7.59-7.66 (m, 4H), 7.71 (dd, J=7.5, 1.5 Hz, 1H), 7.80-7.86 (m, 1H), 8.30 (d, J=8.6 Hz, 1H), 9.14 (s, 1H).

### Examples 9 and 10

### (−)-Benzyl 1-({2-[3-(Dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate and (+)-Benzyl 1-({2-[3-(Dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate

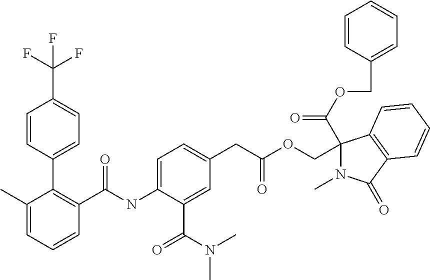 [SMILES: Cc1cccc(C(=O)Nc2ccc(CC(=O)OCC3(C(=O)OCc4ccccc4)c4ccccc4C(=O)N3C)cc2C(=O)N(C)C)c1-c1ccc(C(F)(F)F)cc1]

### Step A: (±)-Benzyl 1-({2-[3-(Dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate

The title compound was prepared following the general procedure for Example 1. 6-Methyl-4′-(trifluoromethyl)biphenyl-2-carboxylic acid and (±)-benzyl 1-(hydroxymethyl)-2-methyl-3-oxoisoindoline-1-carboxylate were used. LCMS (ESI) m/z: 778.5 [M+H] (100%). ¹H NMR (400 MHz, CDCl₃) δ 2.12 (s, 3H), 2.89 (br. s., 3H), 3.04 (s, 3H), 3.09 (br. s., 3H), 3.25-3.35 (m, 2H), 4.64 (d, J=11.9 Hz, 1H), 4.82 (d, J=11.9 Hz, 1H), 5.08 (d, J=12.3 Hz, 1H), 5.20 (d, J=12.1 Hz, 1H), 6.88-6.93 (m, 2H), 7.18-7.24 (m, 2H), 7.30-7.35 (m, 3H), 7.36-7.40 (m, 2H), 7.41-7.50 (m, 6H), 7.61 (d, J=8.0 Hz, 2H), 7.79-7.83 (m, 1H), 8.05 (d, J=8.2 Hz, 1H), 9.06 (s, 1H).

### Step B: (−)-Benzyl 1-({2-[3-(Dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate

(±)-Benzyl 1-({2-[3-(dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate (275 mg, 0.354 mmol) was purified by chiral SFC (Chiralcel OJ-H 10×250 mm column, 90:10 CO₂/MeOH) to give the title compound (105 mg). By analytical chiral SFC (Chiralcel OJ-H 4.6×250 mm column, 90:10 CO₂/MeOH, 2.5 mL/min.), this product was observed to have t_{R}=7.97 min. [α]_{D}=−49° (MeOH, c=1.8).

### Step C: (+)-Benzyl 1-({2-[3-(Dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate

(±)-Benzyl 1-({2-[3-(dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate (275 mg, 0.354 mmol) was purified by chiral SFC (Chiralcel OJ-H 10×250 mm column, 90:10 CO₂/MeOH) to give the title compound (105 mg). By analytical chiral SFC (Chiralcel OJ-H 4.6×250 mm column, 90:10 CO₂/MeOH, 2.5 mL/min.), this product was observed to have t_{R}=10.37 min. [α]_{D}=+52° (MeOH, c=1.8).

### Examples 2-44

The following compounds were prepared following the general procedure for Examples 1, 9, or 10 using analogous starting materials. The appropriate acid, core, and alcohol are substituted and/or resolved as described in the preparations section, are commercially available, or may be prepared by someone skilled in the art.

| | | ¹H NMR | LCMS |
|---|---|---|---|
| Ex. | | (400 MHz, CDCl₃) | (ESI) |
| # | Structure and Name | unless otherwise noted | m/z |
| 2 | 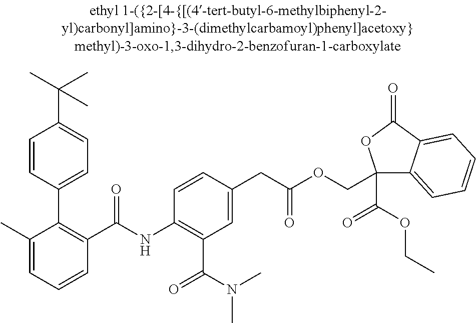 [SMILES: CCOC(=O)C1(COC(=O)Cc2ccc(NC(=O)c3cccc(C)c3-c3ccc(C(C)(C)C)cc3)c(C(=O)N(C)C)c2)OC(=O)c2ccccc21] | δ 1.21 (t, J = 7.1 Hz, 3 H), 1.26 (s, 9 H), 2.15 (s, 3 H), 2.94 (br. s., 3 H), 3.09 (br. s., 3 H), 3.39-3.44 (m, 1 H), 3.44-3.49 (m, 1 H), 4.17 (dq, J = 10.8, 7.1 Hz, 1 H), 4.22 (dq, J = 10.7, 7.1 Hz, 1 H), 4.67 (d, J = 12.0 Hz, 1 H), 4.75 (d, J = 11.9 Hz, 1 H), 6.93 (dd, J = 8.4, 2.1 Hz, 1 H), 6.96 (d, J = 1.9 Hz, 1 H), 7.21- 7.25 (m, 2 H), 7.31 (t, J = 7.3 Hz, 1 H), 7.33-7.39 (m, 3 H), 7.43 (dd, J = 7.2, 1.7 Hz, 1 H), 7.52-7.58 (m, 2 H), 7.64 (td, J = 7.5, 1.2 Hz, 1 H), 7.78 (d, J = 8.4 Hz, 1 H), 7.84 (dt, J = 7.5, 1.0 Hz, 1 H), 8.58 (s, 1 H) | 691 |
| 3 | 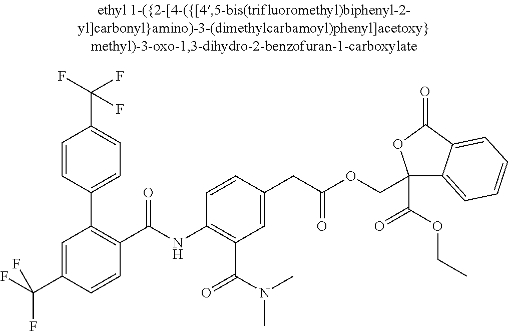 [SMILES: CCOC(=O)C1(COC(=O)Cc2ccc(NC(=O)c3ccc(C(F)(F)F)cc3-c3ccc(C(F)(F)F)cc3)c(C(=O)N(C)C)c2)OC(=O)c2ccccc21] | δ 1.23 (t, J = 7.1 Hz, 3 H), 2.93 (br. s., 3 H), 2.98 (br. s., 3 H), 3.43-3.54 (m, 2 H), 4.18 (dq, J = 10.7, 7.2 Hz, 1 H), 4.24 (dq, J = 10.7, 7.0 Hz, 1 H), 4.66 (d, J = 11.9 Hz, 1 H), 4.79 (d, J = 11.9 Hz, 1 H), 7.03 (d, J = 2.1 Hz, 1 H), 7.07 (dd, J = 8.4, 2.1 Hz, 1 H), 7.56-7.62 (m, 2 H), 7.62-7.71 (m, 6 H), 7.77 (dd, J = 8.1, 1.6 Hz, 1 H), 7.84 (d, J = 7.9 Hz, 1 H), 7.86-7.91 (m, 1 H), 8.29 (d, J = 8.5 Hz, 1 H), 9.43 (s, 1 H) | 757 |
| 4 | 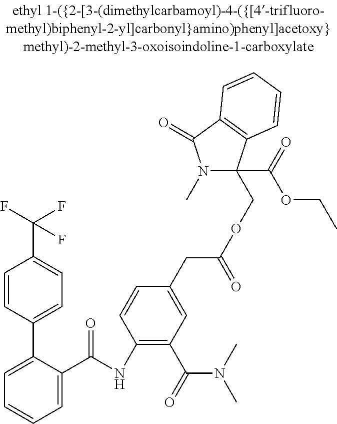 [SMILES: CCOC(=O)C1(COC(=O)Cc2ccc(NC(=O)c3ccccc3-c3ccc(C(F)(F)F)cc3)c(C(=O)N(C)C)c2)c2ccccc2C(=O)N1C] | δ 1.20 (t, J = 7.1 Hz, 3 H), 2.84 (br. s., 3 H), 2.97 (br. s., 3 H), 3.09 (s, 3 H), 3.32-3.43 (m, 2 H), 4.12 (dq, J = 10.8, 7.1 Hz, 1 H), 4.24 (dq, J = 10.7, 7.2 Hz, 1 H), 4.68 (d, J = 11.9 Hz, 1 H), 4.83 (d, J = 11.9 Hz, 1 H), 6.91 (d, J = 2.1 Hz, 1 H), 7.01 (dd, J = 8.6, 2.0 Hz, 1 H), 7.41 (dd, J = 7.6, 1.4 Hz, 1 H), 7.47- 7.58 (m, 5 H), 7.59-7.66 (m, 4 H), 7.71 (dd, J = 7.5, 1.5 Hz, 1 H), 7.80-7.86 (m, 1 H), 8.30 (d, J = 8.6 Hz, 1 H), 9.14 (s, 1 H) | 702 |
| 6 | 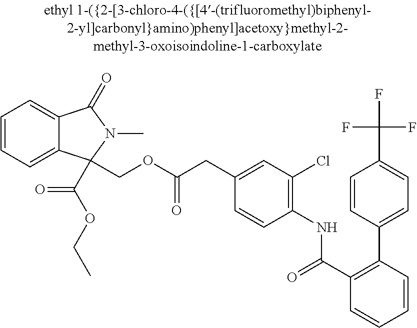 [SMILES: CCOC(=O)C1(COC(=O)Cc2ccc(NC(=O)c3ccccc3-c3ccc(C(F)(F)F)cc3)c(Cl)c2)c2ccccc2C(=O)N1C] | δ 1.20 (t, J = 7.1 Hz, 3 H), 3.06 (s, 3 H), 3.30-3.42 (m, 2 H), 4.12 (dq, J = 10.7, 7.2 Hz, 1 H), 4.24 (dq, J = 10.8, 7.1 Hz, 1 H), 4.68 (d, J = 11.9 Hz, 1 H), 4.84 (d, J = 11.9 Hz, 1 H), 6.89 (s, 1 H), 6.95 (s, 1 H), 7.42-7.72 (m, 11 H), 7.76-7.88 (m, 2 H), 8.29 (d, J = 8.6 Hz, 1 H) | 665 |
| 7 | 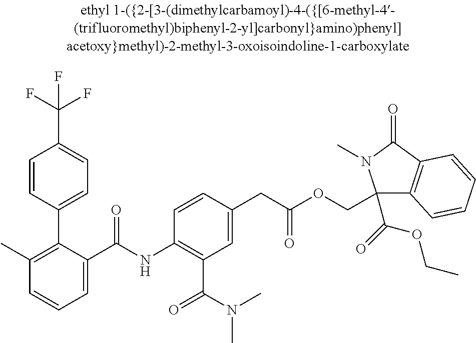 [SMILES: CCOC(=O)C1(COC(=O)Cc2ccc(NC(=O)c3cccc(C)c3-c3ccc(C(F)(F)F)cc3)c(C(=O)N(C)C)c2)c2ccccc2C(=O)N1C] | δ 1.19 (t, J = 7.1 Hz, 3 H), 2.12 (s, 3 H), 2.90 (br. s., 3 H), 3.05 (br. s., 3 H), 3.08 (s, 3 H), 3.30-3.41 (m, 2 H), 4.11 (dq, J = 10.7, 7.1 Hz, 1 H), 4.22 (dq, J = 10.7, 7.1 Hz, 1 H), 4.67 (d, J = 11.9 Hz, 1 H), 4.81 (d, J = 11.9 Hz, 1 H), 6.90-6.97 (m, 2 H), 7.34-7.40 (m, 2 H), 7.42-7.51 (m, 6 H), 7.61 (d, J = 8.0 Hz, 2 H), 7.79- 7.83 (m, 1 H), 8.06 (d, J = 8.4 Hz, 1 H), 9.07 (s, 1 H) | 716 |
| 8 | 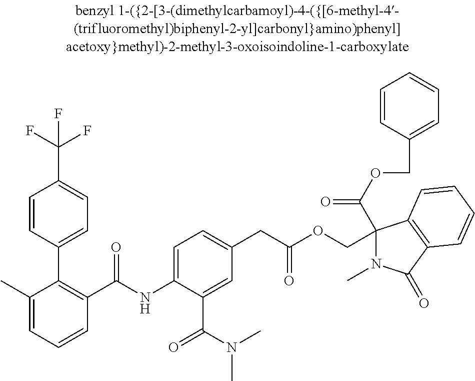 [SMILES: Cc1cccc(C(=O)Nc2ccc(CC(=O)OCC3(C(=O)OCc4ccccc4)c4ccccc4C(=O)N3C)cc2C(=O)N(C)C)c1-c1ccc(C(F)(F)F)cc1] | δ 2.13 (s, 3 H) 2.89 (br. s., 3 H) 3.04 (s, 3 H) 3.09 (br. s., 3 H) 3.24-3.37 (m, 2 H) 4.64 (d, J = 11.92 Hz, 1 H) 4.82 (d, J = 11.92 Hz, 1 H) 5.03-5.13 (m, 1 H) 5.15-5.24 (m, 1 H) 6.87-6.94 (m, 2 H) 7.19-7.23 (m, 2 H) 7.30-7.50 (m, 11 H) 7.61 (d, J = 8.01 Hz, 2 H) 7.79-7.83 (m, 1 H) 8.05 (d, J = 8.21 Hz, 1 H) 9.06 (s, 1 H) | 778 |
| 9 | 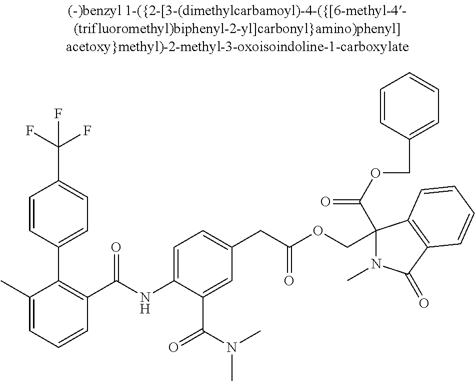 [SMILES: Cc1cccc(C(=O)Nc2ccc(CC(=O)OCC3(C(=O)OCc4ccccc4)c4ccccc4C(=O)N3C)cc2C(=O)N(C)C)c1-c1ccc(C(F)(F)F)cc1] | δ 2.13 (s, 3 H) 2.89 (br. s., 3 H) 3.04 (s, 3 H) 3.09 (br. s., 3 H) 3.24-3.37 (m, 2 H) 4.64 (d, J = 11.92 Hz, 1 H) 4.82 (d, J = 11.92 Hz, 1 H) 5.03-5.13 (m, 1 H) 5.15-5.24 (m, 1 H) 6.87-6.94 (m, 2 H) 7.19-7.23 (m, 2 H) 7.30-7.50 (m, 11 H) 7.61 (d, J = 8.01 Hz, 2 H) 7.79-7.83 (m, 1 H) 8.05 (d, J = 8.21 Hz, 1 H) 9.06 (s, 1 H) | 778 |
| 10 | 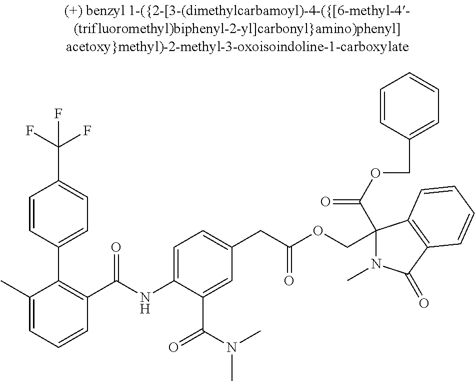 [SMILES: Cc1cccc(C(=O)Nc2ccc(CC(=O)OCC3(C(=O)OCc4ccccc4)c4ccccc4C(=O)N3C)cc2C(=O)N(C)C)c1-c1ccc(C(F)(F)F)cc1] | δ 2.13 (s, 3 H) 2.89 (br. s., 3 H) 3.04 (s, 3 H) 3.09 (br. s., 3 H) 3.24-3.36 (m, 2 H) 4.65 (d, J = 11.90 Hz, 1 H) 4.82 (d, J = 11.90 Hz, 1 H) 5.05-5.11 (m, 1 H) 5.17-5.22 (m, 1 H) 6.88-6.94 (m, 2 H) 7.19-7.24 (m, 2 H) 7.30-7.51 (m, 11 H) 7.61 (d, J = 8.00 Hz, 2 H) 7.78-7.83 (m, 1 H) 8.05 (d, J = 8.19 Hz, 1 H) 9.06 (s, 1 H) | 778 |
| 11 | 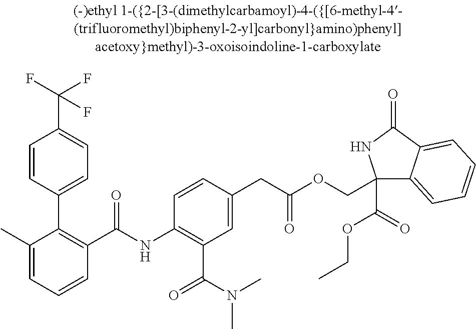 [SMILES: CCOC(=O)C1(COC(=O)Cc2ccc(NC(=O)c3cccc(C)c3-c3ccc(C(F)(F)F)cc3)c(C(=O)N(C)C)c2)NC(=O)c2ccccc21] | δ 1.23 (t, J = 7.2 Hz, 3 H), 2.88 (br. s., 3 H), 2.97 (br. s., 3 H), 3.57 (s, 2 H), 4.16 (dq, J = 10.7, 7.1 Hz, 1 H), 4.14 (d, J = 10.9 Hz, 1 H), 4.20 (dq, J = 10.7, 7.1 Hz, 1 H), 4.91 (d, J = 10.9 Hz, 1 H), 6.53 (s, 1 H), 7.06 (d, J = 2.0 Hz, 1 H), 7.21 (dd, J = 8.6, 2.2 Hz, 1 H), 7.39-7.43 (m, 1 H), 7.50 (td, J = 7.5, 1.5 Hz, 1 H), 7.55 (td, J = 7.5, 1.7 Hz, 1 H), 7.56 (td, J = 7.1, 1.7 Hz, 1 H), 7.59- 7.66 (m, 6 H), 7.69-7.73 (m, 1 H), 7.85 (ddd, J = 7.3, 1.4, 0.8 Hz, 1 H), 8.38 (d, J = 8.6 Hz, 1 H), 9.16 (s, 1 H) | 702 |
| 12 | 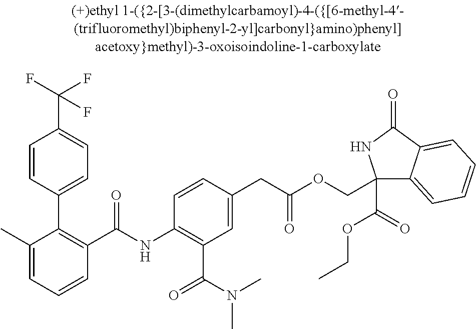 [SMILES: CCOC(=O)C1(COC(=O)Cc2ccc(NC(=O)c3cccc(C)c3-c3ccc(C(F)(F)F)cc3)c(C(=O)N(C)C)c2)NC(=O)c2ccccc21] | δ 1.23 (t, J = 7.2 Hz, 3 H), 2.88 (br. s., 3 H), 2.97 (br. s., 3 H), 3.57 (s, 2 H), 4.16 (dq, J = 10.7, 7.1 Hz, 1 H), 4.14 (d, J = 10.9 Hz, 1 H), 4.20 (dq, J = 10.7, 7.1 Hz, 1 H), 4.91 (d, J = 10.9 Hz, 1 H), 6.53 (s, 1 H), 7.06 (d, J = 2.0 Hz, 1 H), 7.21 (dd, J = 8.6, 2.2 Hz, 1 H), 7.39-7.43 (m, 1 H), 7.50 (td, J = 7.5, 1.5 Hz, 1 H), 7.55 (td, J = 7.5, 1.7 Hz, 1 H), 7.56 (td, J = 7.1, 1.7 Hz, 1 H), 7.59- 7.66 (m, 6 H), 7.69-7.73 (m, 1 H), 7.85 (ddd, J = 7.3, 1.4, 0.8 Hz, 1 H), 8.38 (d, J = 8.6 Hz, 1 H), 9.16 (s, 1 H) | 702 |
| 13 | 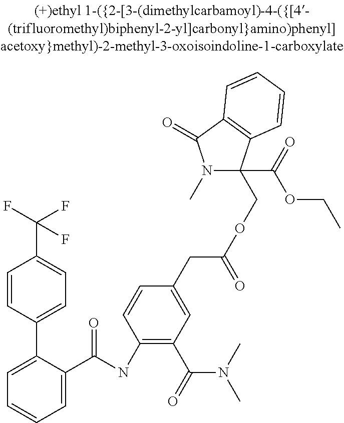 [SMILES: CCOC(=O)C1(COC(=O)Cc2ccc(NC(=O)c3ccccc3-c3ccc(C(F)(F)F)cc3)c(C(=O)N(C)C)c2)c2ccccc2C(=O)N1C] | δ 1.20 (t, J = 7.1 Hz, 3 H), 2.84 (br. s., 3 H), 2.97 (br. s., 3 H), 3.09 (s, 3 H), 3.32-3.43 (m, 2 H), 4.12 (dq, J = 10.8, 7.1 Hz, 1 H), 4.24 (dq, J = 10.7, 7.2 Hz, 1 H), 4.68 (d, J = 11.9 Hz, 1 H), 4.83 (d, J = 11.9 Hz, 1 H), 6.91 (d, J = 2.1 Hz, 1 H), 7.01 (dd, J = 8.6, 2.0 Hz, 1 H), 7.41 (dd, J = 7.6, 1.4 Hz, 1 H), 7.47- 7.58 (m, 5 H), 7.59-7.66 (m, 4 H), 7.71 (dd, J = 7.5, 1.5 Hz, 1 H), 7.80-7.86 (m, 1 H), 8.30 (d, J = 8.6 Hz, 1 H), 9.14 (s, 1 H) | 702 |
| 14 | 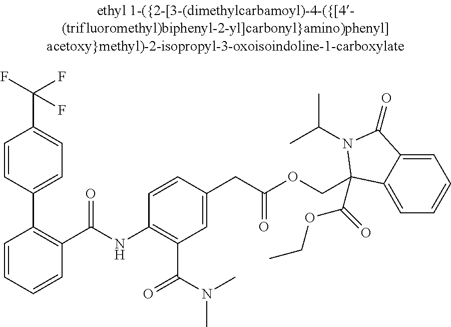 [SMILES: CCOC(=O)C1(COC(=O)Cc2ccc(NC(=O)c3ccccc3-c3ccc(C(F)(F)F)cc3)c(C(=O)N(C)C)c2)c2ccccc2C(=O)N1C(C)C] | δ 1.21 (t, J = 7.1 Hz, 3 H), 1.41 (d, J = 6.8 Hz, 3 H), 1.63 (d, J = 6.7 Hz, 3 H), 2.83 (br. s., 3 H), 2.96 (br. s., 3 H), 3.32-3.37 (m, J = 15.3 Hz, 1 H), 3.37-3.41 (m, J = 15.5 Hz, 1 H), 3.59-3.70 (m, J = 6.8, 6.8, 6.8, 6.8, 6.8, 6.8 Hz, 1 H), 4.13 (dq, J = 10.8, 7.1 Hz, 1 H), 4.22 (dq, J = 10.8, 7.1 Hz, 1 H), 4.64 (d, J = 11.9 Hz, 1 H), 4.80 (d, J = 11.9 Hz, 1 H), 6.89 (d, J = 2.2 Hz, 1 H), 7.00 (dd, J = 8.5, 2.1 Hz, 1 H), 7.36-7.42 (m, 2 H), 7.45-7.52 (m, 3 H), 7.55 (td, J = 7.6, 1.6 Hz, 1 H), 7.60-7.65 (m, 4 H), 7.70 (dd, J = 7.4, 1.6 Hz, 1 H), 7.74-7.79 (m, 1 H), 8.30 (d, J = 8.5 Hz, 1 H), 9.16 (s, 1 H) | 730 |
| 15 | 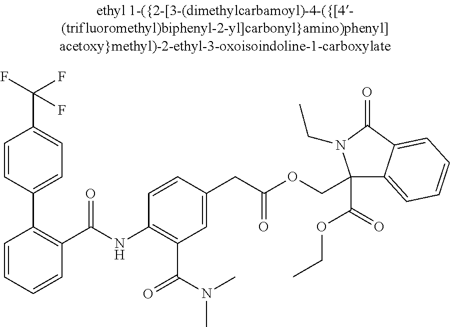 [SMILES: CCOC(=O)C1(COC(=O)Cc2ccc(NC(=O)c3ccccc3-c3ccc(C(F)(F)F)cc3)c(C(=O)N(C)C)c2)c2ccccc2C(=O)N1CC] | δ 1.18 (t, J = 7.1 Hz, 3 H), 1.24 (t, J = 7.2 Hz, 3 H), 2.84 (br. s., 3 H), 2.96 (br. s., 3 H), 3.38 (dq, J = 14.4, 7.2 Hz, 1 H), 3.37 (d, J = 15.4 Hz, 1 H), 3.42 (d, J = 15.3 Hz, 1 H), 3.72 (dq, J = 14.4, 7.2 Hz, 1 H), 4.10 (dq, J = 10.7, 7.1 Hz, 1 H), 4.20 (dq, J = 10.7, 7.1 Hz, 1 H), 4.70 (d, J = 11.9 Hz, 1 H), 4.78 (d, J = 11.8 Hz, 1 H), 6.92 (d, J = 2.1 Hz, 1 H), 7.03 (dd, J = 8.6, 1.9 Hz, 1 H), 7.39-7.42 (m, 1 H), 7.42-7.47 (m, 1 H), 7.48-7.52 (m, 3 H), 7.55 (td, J = 7.5, 1.6 Hz, 1 H), 7.60-7.65 (m, 4 H), 7.69-7.72 (m, 1 H), 7.79-7.84 (m, 1 H), 8.31 (d, J = 8.5 Hz, 1 H), 9.15 (s, 1 H) | 716 |
| 16 | 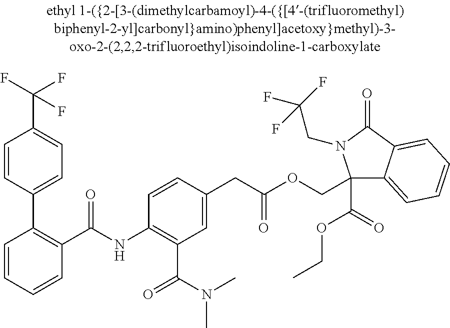 [SMILES: CCOC(=O)C1(COC(=O)Cc2ccc(NC(=O)c3ccccc3-c3ccc(C(F)(F)F)cc3)c(C(=O)N(C)C)c2)c2ccccc2C(=O)N1CC(F)(F)F] | δ 1.18 (t, 3 H), 2.84 (br. s., 3 H), 2.96 (br. s., 3 H), 3.38- 3.42 (m, J = 15.8 Hz, 1 H), 3.42-3.46 (m, J = 15.7 Hz, 1 H), 3.97 (dq, J = 15.9, 9.1 Hz, 1 H), 4.11 (dq, J = 10.8, 7.1 Hz, 1 H), 4.18 (dq, J = 10.8, 7.1 Hz, 1 H), 4.44 (dq, J = 15.9, 9.1 Hz, 1 H), 4.72 (d, J = 12.0 Hz, 1 H), 4.83 (d, J = 12.1 Hz, 1 H), 6.92 (d, J = 2.0 Hz, 1 H), 7.03 (dd, J = 8.6, 2.0 Hz, 1 H), 7.41 (dd, J = 7.3, 1.5 Hz, 1 H), 7.47- 7.59 (m, 4 H), 7.57 (td, J = 7.2, 1.6 Hz, 1 H), 7.59- 7.66 (m, 4 H), 7.71 (dd, J = 7.6, 1.4 Hz, 1 H), 7.83- 7.89 (m, 1 H), 8.31 (d, J = 8.6 Hz, 1 H), 9.17 (s, 1 H) | 770 |
| 17 | 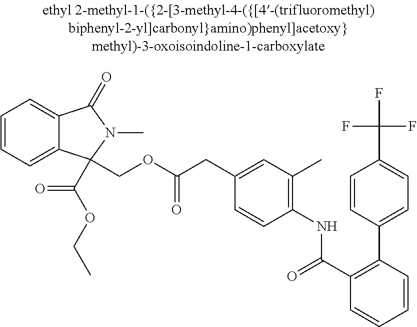 [SMILES: CCOC(=O)C1(COC(=O)Cc2ccc(NC(=O)c3ccccc3-c3ccc(C(F)(F)F)cc3)c(C)c2)c2ccccc2C(=O)N1C] | δ 1.20 (t, J = 7.1 Hz, 3 H), 1.69 (s, 3 H), 3.03 (s, 3 H), 3.36 (s, 2 H), 4.05-4.16 (m, 1 H), 4.22 (dq, J = 10.7, 7.2 Hz, 1 H), 4.63 (d, J = 11.7 Hz, 1 H), 4.83 (d, J = 11.9 Hz, 1 H), 6.75-6.88 (m, 3 H), 7.42-7.62 (m, 6 H), 7.63-7.73 (m, 5 H), 7.75-7.80 (m, 1 H), 7.83 (dd, J = 7.4, 1.2 Hz, 1 H) | 645 |
| 18 | 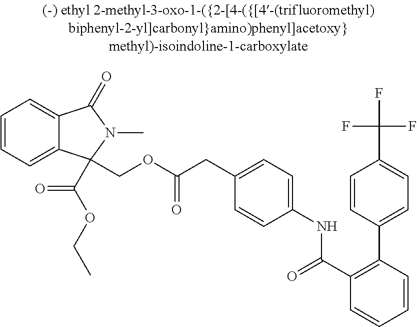 [SMILES: CCOC(=O)C1(COC(=O)Cc2ccc(NC(=O)c3ccccc3-c3ccc(C(F)(F)F)cc3)cc2)c2ccccc2C(=O)N1C] | δ 1.20 (t, J = 7.2 Hz, 3 H), 3.02 (s, 3 H), 3.39 (s, 2 H), 4.05-4.15 (m, 1 H), 4.22 (dq, J = 10.8, 7.1 Hz, 1 H), 4.63 (d, J = 11.9 Hz, 1 H), 4.83 (d, J = 11.9 Hz, 1 H), 6.90 (d, J = 8.6 Hz, 2 H), 7.08-7.17 (m, 3 H), 7.41-7.63 (m, 8 H), 7.66-7.71 (m, 2 H), 7.77 (dd, J = 17.9, 7.1 Hz, 2 H), 7.77 (dd, J = 15.5, 7.3 Hz, 4 H) | 631 |
| 19 | 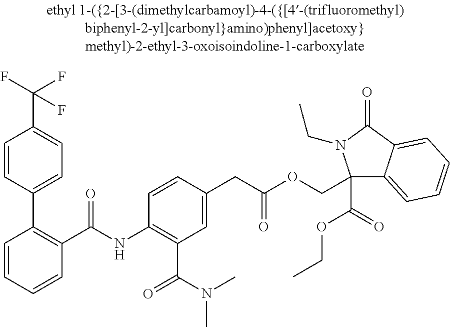 [SMILES: CCOC(=O)C1(COC(=O)Cc2ccc(NC(=O)c3ccccc3-c3ccc(C(F)(F)F)cc3)c(C(=O)N(C)C)c2)c2ccccc2C(=O)N1CC] | δ 1.18 (t, J = 7.1 Hz, 3 H), 1.24 (t, J = 7.2 Hz, 3 H), 2.84 (br. s., 3 H), 2.96 (br. s., 3 H), 3.38 (dq, J = 14.4, 7.2 Hz, 1 H), 3.37 (d, J = 15.4 Hz, 1 H), 3.42 (d, J = 15.3 Hz, 1 H), 3.72 (dq, J = 14.4, 7.2 Hz, 1 H), 4.10 (dq, J = 10.7, 7.1 Hz, 1 H), 4.20 (dq, J = 10.7, 7.1 Hz, 1 H), 4.70 (d, J = 11.9 Hz, 1 H), 4.78 (d, J = 11.8 Hz, 1 H), 6.92 (d, J = 2.1 Hz, 1 H), 7.03 (dd, J = 8.6, 1.9 Hz, 1 H), 7.39-7.42 (m, 1 H), 7.42-7.47 (m, 1 H), 7.48-7.52 (m, 3 H), 7.55 (td, J = 7.5, 1.6 Hz, 1 H), 7.60-7.65 (m, 4 H), 7.69-7.72 (m, 1 H), 7.79-7.84 (m, 1 H), 8.31 (d, J = 8.5 Hz, 1 H), 9.15 (s, 1 H) | 716 |
| 20 | 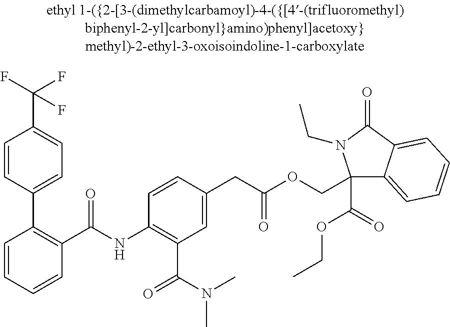 [SMILES: CCOC(=O)C1(COC(=O)Cc2ccc(NC(=O)c3ccccc3-c3ccc(C(F)(F)F)cc3)c(C(=O)N(C)C)c2)c2ccccc2C(=O)N1CC] | δ 1.18 (t, J = 7.1 Hz, 3 H), 1.24 (t, J = 7.2 Hz, 3 H), 2.84 (br. s., 3 H), 2.96 (br. s., 3 H), 3.38 (dq, J = 14.4, 7.2 Hz, 1 H), 3.37 (d, J = 15.4 Hz, 1 H), 3.42 (d, J = 15.3 Hz, 1 H), 3.72 (dq, J = 14.4, 7.2 Hz, 1 H), 4.10 (dq, J = 10.7, 7.1 Hz, 1 H), 4.20 (dq, J = 10.7, 7.1 Hz, 1 H), 4.70 (d, J = 11.9 Hz, 1 H), 4.78 (d, J = 11.8 Hz, 1 H), 6.92 (d, J = 2.1 Hz, 1 H), 7.03 (dd, J = 8.6, 1.9 Hz, 1 H), 7.39-7.42 (m, 1 H), 7.42-7.47 (m, 1 H), 7.48-7.52 (m, 3 H), 7.55 (td, J = 7.5, 1.6 Hz, 1 H), 7.60-7.65 (m, 4 H), 7.69-7.72 (m, 1 H), 7.79-7.84 (m, 1 H), 8.31 (d, J = 8.5 Hz, 1 H), 9.15 (s, 1 H) | 716 |
| 21 | 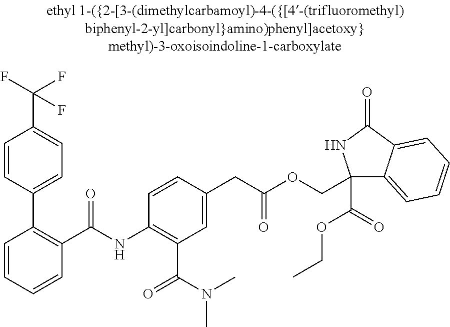 [SMILES: CCOC(=O)C1(COC(=O)Cc2ccc(NC(=O)c3ccccc3-c3ccc(C(F)(F)F)cc3)c(C(=O)N(C)C)c2)NC(=O)c2ccccc21] | δ 1.23 (t, J = 7.2 Hz, 3 H), 2.88 (br. s., 3 H), 2.97 (br. s., 3 H), 3.57 (s, 2 H), 4.16 (dq, J = 10.7, 7.1 Hz, 1 H), 4.14 (d, J = 10.9 Hz, 1 H), 4.20 (dq, J = 10.7, 7.1 Hz, 1 H), 4.91 (d, J = 10.9 Hz, 1 H), 6.53 (s, 1 H), 7.06 (d, J = 2.0 Hz, 1 H), 7.21 (dd, J = 8.6, 2.2 Hz, 1 H), 7.39-7.43 (m, 1 H), 7.50 (td, J = 7.5, 1.5 Hz, 1 H), 7.55 (td, J = 7.5, 1.7 Hz, 1 H), 7.56 (td, J = 7.1, 1.7 Hz, 1 H), 7.59- 7.66 (m, 6 H), 7.69-7.73 (m, 1 H), 7.85 (ddd, J = 7.3, 1.4, 0.8 Hz, 1 H), 8.38 (d, J = 8.6 Hz, 1 H), 9.16 (s, 1 H) | 688 |
| 22 | 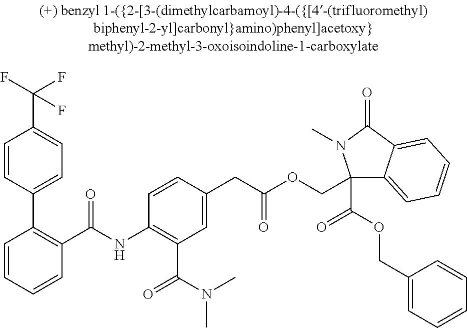 [SMILES: CN(C)C(=O)c1cc(CC(=O)OCC2(C(=O)OCc3ccccc3)c3ccccc3C(=O)N2C)ccc1NC(=O)c1ccccc1-c1ccc(C(F)(F)F)cc1] | δ 2.82 (br. s., 3 H), 2.96 (br. s., 3 H), 3.05 (s, 3 H), 3.26- 3.32 (m, 1 H), 3.32-3.38 (m, 1 H), 4.65 (d, J = 11.9 Hz, 1 H), 4.83 (d, J = 11.9 Hz, 1 H), 5.08 (d, J = 12.1 Hz, 1 H), 5.20 (d, J = 12.1 Hz, 1 H), 6.88 (d, J = 1.6 Hz, 1 H), 6.98 (dd, J = 8.4, 1.6 Hz, 1 H), 7.18-7.25 (m, 2 H), 7.30-7.36 (m, 3 H), 7.37-7.58 (m, 6 H), 7.58-7.66 (m, 4 H), 7.70 (dd, J = 7.3, 0.9 Hz, 1 H), 7.83 (m, J = 5.4, 3.0 Hz, 1 H), 8.29 (d, J = 8.4 Hz, 1 H), 9.14 (s, 1 H) | 764 |
| 23 | 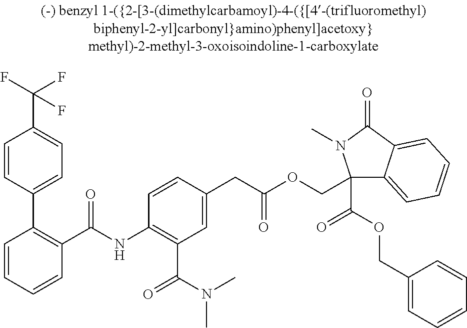 [SMILES: CN(C)C(=O)c1cc(CC(=O)OCC2(C(=O)OCc3ccccc3)c3ccccc3C(=O)N2C)ccc1NC(=O)c1ccccc1-c1ccc(C(F)(F)F)cc1] | δ 2.82 (br. s., 3 H), 2.96 (br. s., 3 H), 3.05 (s, 3 H), 3.26- 3.32 (m, 1 H), 3.32-3.38 (m, 1 H), 4.65 (d, J = 11.9 Hz, 1 H), 4.83 (d, J = 11.9 Hz, 1 H), 5.08 (d, J = 12.1 Hz, 1 H), 5.20 (d, J = 12.1 Hz, 1 H), 6.88 (d, J = 1.6 Hz, 1 H), 6.98 (dd, J = 8.4, 1.6 Hz, 1 H), 7.18-7.25 (m, 2 H), 7.30-7.36 (m, 3 H), 7.37-7.58 (m, 6 H), 7.58-7.66 (m, 4 H), 7.70 (dd, J = 7.3, 0.9 Hz, 1 H), 7.83 (m, J = 5.4, 3.0 Hz, 1 H), 8.29 (d, J = 8.4 Hz, 1 H), 9.14 (s, 1 H) | 764 |
| 24 | 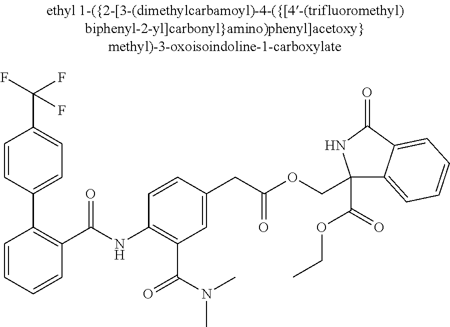 [SMILES: CCOC(=O)C1(COC(=O)Cc2ccc(NC(=O)c3ccccc3-c3ccc(C(F)(F)F)cc3)c(C(=O)N(C)C)c2)NC(=O)c2ccccc21] | δ 1.23 (t, J = 7.2 Hz, 3 H), 2.88 (br. s., 3 H), 2.97 (br. s., 3 H), 3.57 (s, 2 H), 4.16 (dq, J = 10.7, 7.1 Hz, 1 H), 4.14 (d, J = 10.9 Hz, 1 H), 4.20 (dq, J = 10.7, 7.1 Hz, 1 H), 4.91 (d, J = 10.9 Hz, 1 H), 6.53 (s, 1 H), 7.06 (d, J = 2.0 Hz, 1 H), 7.21 (dd, J = 8.6, 2.2 Hz, 1 H), 7.39-7.43 (m, 1 H), 7.50 (td, J = 7.5, 1.5 Hz, 1 H), 7.55 (td, J = 7.5, 1.7 Hz, 1 H), 7.56 (td, J = 7.1, 1.7 Hz, 1 H), 7.59- 7.66 (m, 6 H), 7.69-7.73 (m, 1 H), 7.85 (ddd, J = 7.3, 1.4, 0.8 Hz, 1 H), 8.38 (d, J = 8.6 Hz, 1 H), 9.16 (s, 1 H) | 688 |
| 25 | 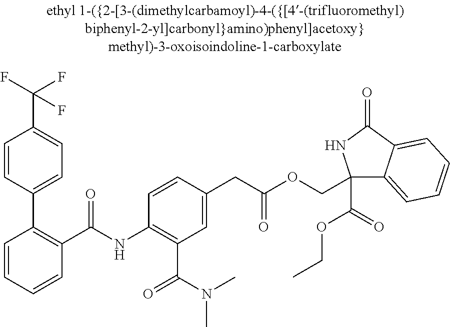 [SMILES: CCOC(=O)C1(COC(=O)Cc2ccc(NC(=O)c3ccccc3-c3ccc(C(F)(F)F)cc3)c(C(=O)N(C)C)c2)NC(=O)c2ccccc21] | δ 1.23 (t, J = 7.2 Hz, 3 H), 2.88 (br. s., 3 H), 2.97 (br. s., 3 H), 3.57 (s, 2 H), 4.16 (dq, J = 10.7, 7.1 Hz, 1 H), 4.14 (d, J = 10.9 Hz, 1 H), 4.20 (dq, J = 10.7, 7.1 Hz, 1 H), 4.91 (d, J = 10.9 Hz, 1 H), 6.53 (s, 1 H), 7.06 (d, J = 2.0 Hz, 1 H), 7.21 (dd, J = 8.6, 2.2 Hz, 1 H), 7.39-7.43 (m, 1 H), 7.50 (td, J = 7.5, 1.5 Hz, 1 H), 7.55 (td, J = 7.5, 1.7 Hz, 1 H), 7.56 (td, J = 7.1, 1.7 Hz, 1 H), 7.59- 7.66 (m, 6 H), 7.69-7.73 (m, 1 H), 7.85 (ddd, J = 7.3, 1.4, 0.8 Hz, 1 H), 8.38 (d, J = 8.6 Hz, 1 H), 9.16 (s, 1 H) | 688 |
| 27 | 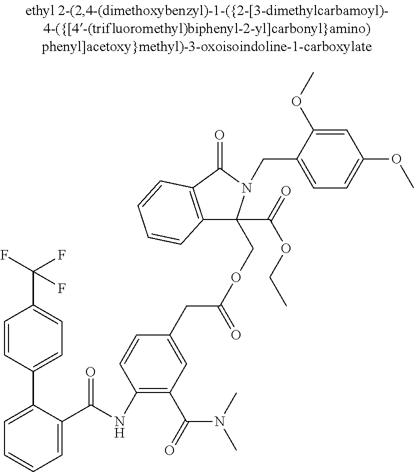 [SMILES: CCOC(=O)C1(COC(=O)Cc2ccc(NC(=O)c3ccccc3-c3ccc(C(F)(F)F)cc3)c(C(=O)N(C)C)c2)c2ccccc2C(=O)N1Cc1ccc(OC)cc1OC] | δ 0.99 (2 H, t, J = 7.1 Hz), 0.98 (1 H, s), 1.24 (1 H, td, J = 7.1, 1.0 Hz), 1.58 (1 H, br. s.), 2.78 (2 H, br. s.), 2.99 (3 H, s), 3.05 (1 H, d, J = 15.0 Hz), 3.72 (2 H, s), 3.78 (3 H, s), 3.86 (1 H, ddd, J = 18.1, 7.5, 7.4 Hz), 4.69 (1 H, d, J = 7.0 Hz), 4.66 (1 H, d, J = 3.1 Hz), 4.79 (1 H, d, J = 3.7 Hz), 4.76 (1 H, s), 6.38 (2 H, m), 6.75 (1 H, d, J = 1.6 Hz), 6.85 (1 H, d, J = 8.6 Hz), 7.24 (3 H, d, J = 1.0 Hz), 7.35 (3 H, m), 7.48 (4 H, m), 7.59 (3 H, s), 7.67 (1 H, d, J = 7.4 Hz), 7.84 (1 H, d, J = 5.5 Hz), 8.22 (1 H, d, J = 8.6 Hz), 9.12 (1 H, s) | 838 |
| 28 | 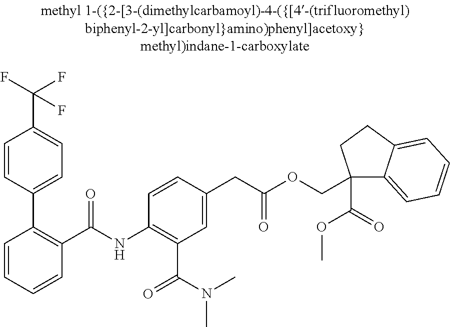 [SMILES: COC(=O)C1(COC(=O)Cc2ccc(NC(=O)c3ccccc3-c3ccc(C(F)(F)F)cc3)c(C(=O)N(C)C)c2)CCc2ccccc21] | δ 2.08 (ddd, J = 13.3, 8.6, 5.9 Hz, 1 H), 2.68 (ddd, J = 13.3, 8.9, 6.1 Hz, 1 H), 2.86 (br. s., 3 H), 2.88-2.95 (m, 1 H), 2.96 (br. s., 3 H), 3.07 (ddd, J = 16.0, 8.9, 5.9 Hz, 1 H), 3.55 (s, 2 H), 3.66 (s, 3 H), 4.30 (d, J = 10.8 Hz, 1 H), 4.52 (d, J = 10.7 Hz, 1 H), 7.07 (d, J = 2.0 Hz, 1 H), 7.14- 7.20 (m, 1 H), 7.21-7.28 (m, 4 H), 7.41 (dd, J = 7.5, 1.3 Hz, 1 H), 7.50 (td, J = 7.4, 1.5 Hz, 1 H), 7.55 (td, J = 7.4, 1.6 Hz, 1 H), 7.60-7.65 (m, 4 H), 7.71 (dd, J = 7.4, 1.4 Hz, 1 H), 8.38 (d, J = 8.5 Hz, 1 H), 9.18 (s, 1 H) | 659 |
| 29 | 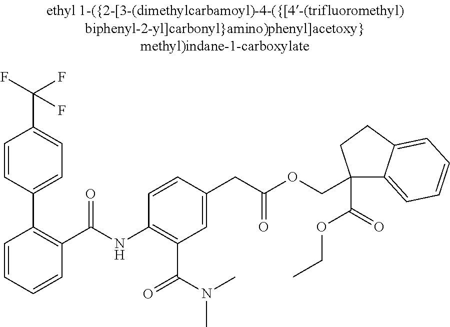 [SMILES: CCOC(=O)C1(COC(=O)Cc2ccc(NC(=O)c3ccccc3-c3ccc(C(F)(F)F)cc3)c(C(=O)N(C)C)c2)CCc2ccccc21] | δ 1.20 (t, J = 7.1 Hz, 3 H), 2.08 (ddd, J = 13.3, 8.6, 5.7 Hz, 1 H), 2.70 (ddd, J = 13.3, 9.0, 6.2 Hz, 1 H), 2.86 (br. s., 3 H), 2.92 (ddd, J = 16.2, 8.6, 6.2 Hz, 1 H), 2.96 (br. s., 3 H), 3.07 (ddd, J = 15.9, 9.0, 5.7 Hz, 1 H), 3.54 (s, 2 H), 4.11 (dq, J = 10.8, 7.1 Hz, 1 H), 4.15 (dq, J = 10.8, 7.1 Hz, 1 H), 4.28 (d, J = 10.7 Hz, 1 H), 4.54 (d, J = 10.7 Hz, 1 H), 7.07 (d, J = 2.0 Hz, 1 H), 7.14- 7.20 (m, 1 H), 7.21-7.25 (m, 3 H), 7.29 (d, J = 7.5 Hz, 1 H), 7.41 (dd, J = 7.5, 1.3 Hz, 1 H), 7.50 (td, J = 7.4, 1.5 Hz, 1 H), 7.55 (td, J = 7.4, 1.6 Hz, 1 H), 7.60-7.65 (m, 4 H), 7.69-7.72 (m, 1 H), 8.37 (d, J = 8.6 Hz, 1 H), 9.18 (s, 1 H) | 673 |
| 30 | 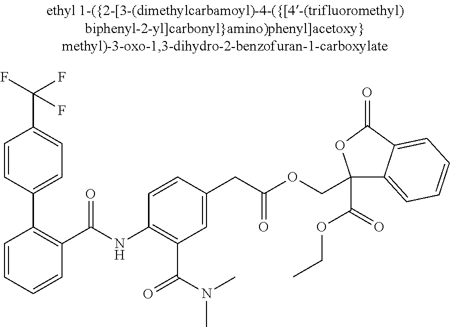 [SMILES: CCOC(=O)C1(COC(=O)Cc2ccc(NC(=O)c3ccccc3-c3ccc(C(F)(F)F)cc3)c(C(=O)N(C)C)c2)OC(=O)c2ccccc21] | δ 1.22 (t, J = 7.1 Hz, 3 H), 2.90 (br. s., 3 H), 2.98 (br. s., 3 H), 3.43-3.52 (m, 2 H), 4.18 (dq, J = 10.7, 7.1 Hz, 1 H), 4.24 (dq, J = 10.7, 7.1 Hz, 1 H), 4.66 (d, J = 11.9 Hz, 1 H), 4.78 (d, J = 11.9 Hz, 1 H), 7.00 (d, J = 2.0 Hz, 1 H), 7.06 (dd, J = 8.5, 2.1 Hz, 1 H), 7.41 (dd, J = 7.5, 1.2 Hz, 1 H), 7.50 (td, J = 7.5, 1.5 Hz, 1 H), 7.55 (td, J = 7.5, 1.7 Hz, 1 H), 7.57 (dt, J = 7.6, 1.0 Hz, 1 H), 7.59 (td, J = 7.5, 1.0 Hz, 1 H), 762- 7.65 (m, 4 H), 7.67 (td, J = 7.5, 1.2 Hz, 1 H), 7.70- 7.72 (m, 1 H), 7.88 (dt, J = 7.6, 1.0 Hz, 1 H), 8.31 (d, J = 8.5 Hz, 1 H), 9.23 (s, 1 H) | 689 |
| 31 | 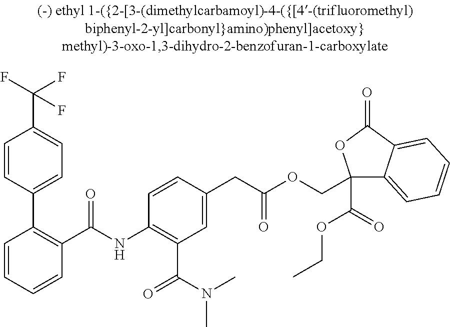 [SMILES: CCOC(=O)C1(COC(=O)Cc2ccc(NC(=O)c3ccccc3-c3ccc(C(F)(F)F)cc3)c(C(=O)N(C)C)c2)OC(=O)c2ccccc21] | δ 1.22 (t, J = 7.1 Hz, 3 H), 2.90 (br. s., 3 H), 2.98 (br. s., 3 H), 3.43-3.52 (m, 2 H), 4.18 (dq, J = 10.7, 7.1 Hz, 1 H), 4.24 (dq, J = 10.7, 7.1 Hz, 1 H), 4.66 (d, J = 11.9 Hz, 1 H), 4.78 (d, J = 11.9 Hz, 1 H), 7.00 (d, J = 2.0 Hz, 1 H), 7.06 (dd, J = 8.5, 2.1 Hz, 1 H), 7.41 (dd, J = 7.5, 1.2 Hz, 1 H), 7.50 (td, J = 7.5, 1.5 Hz, 1 H), 7.55 (td, J = 7.5, 1.7 Hz, 1 H), 7.57 (dt, J = 7.6, 1.0 Hz, 1 H), 7.59 (td, J = 7.5, 1.0 Hz, 1 H), 7.62- 7.65 (m, 4 H), 7.67 (td, J = 7.5, 1.2 Hz, 1 H), 7.70- 7.72 (m, 1 H), 7.88 (dt, J = 7.6, 1.0 Hz, 1 H), 8.31 (d, J = 8.5 Hz, 1 H), 9.23 (s, 1 H) | 689 |
| 32 | 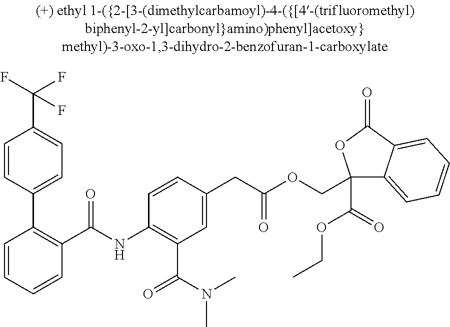 [SMILES: CCOC(=O)C1(COC(=O)Cc2ccc(NC(=O)c3ccccc3-c3ccc(C(F)(F)F)cc3)c(C(=O)N(C)C)c2)OC(=O)c2ccccc21] | δ 1.22 (t, J = 7.1 Hz, 3 H), 2.90 (br. s., 3 H), 2.98 (br. s., 3 H), 3.43-3.52 (m, 2 H), 4.18 (dq, J = 10.7, 7.1 Hz, 1 H), 4.24 (dq, J = 10.7, 7.1 Hz, 1 H), 4.66 (d, J = 11.9 Hz, 1 H), 4.78 (d, J = 11.9 Hz, 1 H), 7.00 (d, J = 2.0 Hz, 1 H), 7.06 (dd, J = 8.5, 2.1 Hz, 1 H), 7.41 (dd, J = 7.5, 1.2 Hz, 1 H), 7.50 (td, J = 7.5, 1.5 Hz, 1 H), 7.55 (td, J = 7.5, 1.7 Hz, 1 H), 7.57 (dt, J = 7.6, 1.0 Hz, 1 H), 7.59 (td, J = 7.5, 1.0 Hz, 1 H), 7.62- 7.65 (m, 4 H), 7.67 (td, J = 7.5, 1.2 Hz, 1 H), 7.70- 7.72 (m, 1 H), 7.88 (dt, J = 7.6, 1.0 Hz, 1 H), 8.31 (d, J = 8.5 Hz, 1 H), 9.23 (s, 1 H) | 689 |
| 33 | 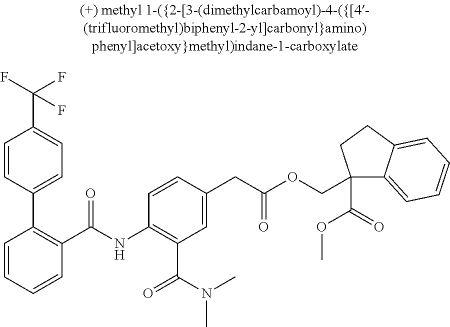 [SMILES: COC(=O)C1(COC(=O)Cc2ccc(NC(=O)c3ccccc3-c3ccc(C(F)(F)F)cc3)c(C(=O)N(C)C)c2)CCc2ccccc21] | δ 2.08 (ddd, J = 13.3, 8.6, 5.9 Hz, 1 H), 2.68 (ddd, J = 13.3, 8.9, 6.1 Hz, 1 H), 2.86 (br. s., 3 H), 2.88-2.95 (m, 1 H), 2.96 (br. s., 3 H), 3.07 (ddd, J = 16.0, 8.9, 5.9 Hz, 1 H), 3.55 (s, 2 H), 3.66 (s, 3 H), 4.30 (d, J = 10.8 Hz, 1 H), 4.52 (d, J = 10.7 Hz, 1 H), 7.07 (d, J = 2.0 Hz, 1 H), 7.14- 7.20 (m, 1 H), 7.21-7.28 (m, 4 H), 7.41 (dd, J = 7.5, 1.3 Hz, 1 H), 7.50 (td, J = 7.4, 1.5 Hz, 1 H), 7.55 (td, J = 7.4, 1.6 Hz, 1 H), 7.60-7.65 (m, 4 H), 7.71 (dd, J = 7.4, 1.4 Hz, 1 H), 8.38 (d, J = 8.5 Hz, 1 H), 9.18 (s, 1 H) | 659 |
| 34 | 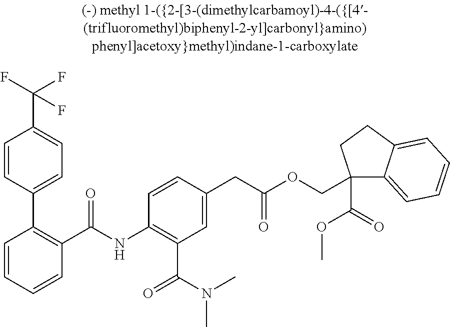 [SMILES: COC(=O)C1(COC(=O)Cc2ccc(NC(=O)c3ccccc3-c3ccc(C(F)(F)F)cc3)c(C(=O)N(C)C)c2)CCc2ccccc21] | δ 2.08 (ddd, J = 13.3, 8.6, 5.9 Hz, 1 H), 2.68 (ddd, J = 13.3, 8.9, 6.1 Hz, 1 H), 2.86 (br. s., 3 H), 2.88-2.95 (m, 1 H), 2.96 (br. s., 3 H), 3.07 (ddd, J = 16.0, 8.9, 5.9 Hz, 1 H), 3.55 (s, 2 H), 3.66 (s, 3 H), 4.30 (d, J = 10.8 Hz, 1 H), 4.52 (d, J = 10.7 Hz, 1 H), 7.07 (d, J = 2.0 Hz, 1 H), 7.14- 7.20 (m, 1 H), 7.21-7.28 (m, 4 H), 7.41 (dd, J = 7.5, 1.3 Hz, 1 H), 7.50 (td, J = 7.4, 1.5 Hz, 1 H), 7.55 (td, J = 7.4, 1.6 Hz, 1 H), 7.60-7.65 (m, 4 H), 7.71 (dd, J = 7.4, 1.4 Hz, 1 H), 8.38 (d, J = 8.5 Hz, 1 H), 9.18 (s, 1 H) | 659 |
| 35 | 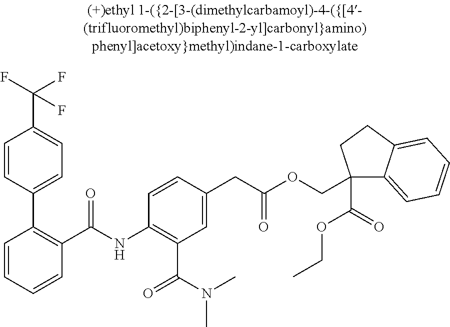 [SMILES: CCOC(=O)C1(COC(=O)Cc2ccc(NC(=O)c3ccccc3-c3ccc(C(F)(F)F)cc3)c(C(=O)N(C)C)c2)CCc2ccccc21] | δ 1.20 (t, J = 7.1 Hz, 3 H), 2.08 (ddd, J = 13.3, 8.6, 5.7 Hz, 1 H), 2.70 (ddd, J = 13.3, 9.0, 6.2 Hz, 1 H), 2.86 (br. s., 3 H), 2.92 (ddd, J = 16.2, 8.6, 6.2 Hz, 1 H), 2.96 (br. s., 3 H), 3.07 (ddd, J = 15.9, 9.0, 5.7 Hz, 1 H), 3.54 (s, 2 H), 4.11 (dq, J = 10.8, 7.1 Hz, 1 H), 4.15 (dq, J = 10.8, 7.1 Hz, 1 H), 4.28 (d, J = 10.7 Hz, 1 H), 4.54 (d, J = 10.7 Hz, 1 H), 7.07 (d, J = 2.0 Hz, 1 H), 7.14- 7.20 (m, 1 H), 7.21-7.25 (m, 3 H), 7.29 (d, J = 7.5 Hz, 1 H), 7.41 (dd, J = 7.5, 1.3 Hz, 1 H), 7.50 (td, J = 7.4, 1.5 Hz, 1 H), 7.55 (td, J = 7.4, 1.6 Hz, 1 H), 7.60-7.65 (m, 4 H), 7.69-7.72 (m, 1 H), 8.37 (d, J = 8.6 Hz, 1 H), 9.18 (s, 1 H) | 673 |
| 36 | 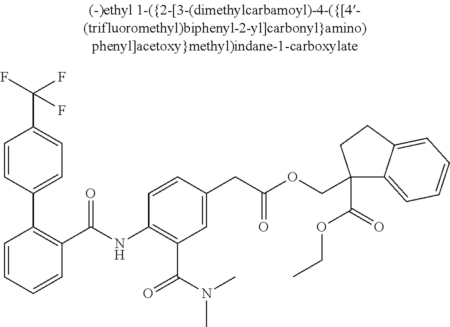 [SMILES: CCOC(=O)C1(COC(=O)Cc2ccc(NC(=O)c3ccccc3-c3ccc(C(F)(F)F)cc3)c(C(=O)N(C)C)c2)CCc2ccccc21] | δ 1.20 (t, J = 7.1 Hz, 3 H), 2.08 (ddd, J = 13.3, 8.6, 5.7 Hz, 1 H), 2.70 (ddd, J = 13.3, 9.0, 6.2 Hz, 1 H), 2.86 (br. s., 3 H), 2.92 (ddd, J = 16.2, 8.6, 6.2 Hz, 1 H), 2.96 (br. s., 3 H), 3.07 (ddd, J = 15.9, 9.0, 5.7 Hz, 1 H), 3.54 (s, 2 H), 4.11 (dq, J = 10.8, 7.1 Hz, 1 H), 4.15 (dq, J = 10.8, 7.1 Hz, 1 H), 4.28 (d, J = 10.7 Hz, 1 H), 4.54 (d, J = 10.7 Hz, 1 H), 7.07 (d, J = 2.0 Hz, 1 H), 7.14- 7.20 (m, 1 H), 7.21-7.25 (m, 3 H), 7.29 (d, J = 7.5 Hz, 1 H), 7.41 (dd, J = 7.5, 1.3 Hz, 1 H), 7.50 (td, J = 7.4, 1.5 Hz, 1 H), 7.55 (td, J = 7.4, 1.6 Hz, 1 H), 7.60-7.65 (m, 4 H), 7.69-7.72 (m, 1 H), 8.37 (d, J = 8.6 Hz, 1 H), 9.18 (s, 1 H) | 673 |
| 37 | 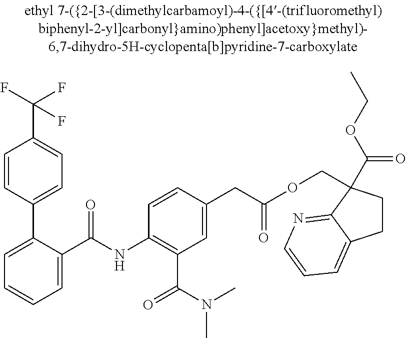 [SMILES: CCOC(=O)C1(COC(=O)Cc2ccc(NC(=O)c3ccccc3-c3ccc(C(F)(F)F)cc3)c(C(=O)N(C)C)c2)CCc2cccnc21] | δ 1.18 (t, 3 H), 2.03-2.15 (m, 1 H), 2.63 (ddd, J = 13.4, 8.9, 4.6 Hz, 1 H), 2.74-3.00 (m, 7 H), 3.00-3.12 (m, 1 H), 3.49 (s, 2 H), 4.10-4.19 (m, 2 H), 4.52 (d, J = 10.9 Hz, 1 H), 4.77 (d, J = 11.1 Hz, 1 H), 7.02 (d, J = 2.0 Hz, 1 H), 7.08- 7.15 (m, 2 H), 7.40 (dd, J = 7.7, 1.3 Hz, 1 H), 7.46- 7.58 (m, 3 H), 7.58-7.66 (m, 4 H), 7.70 (dd, J = 7.6, 1.6 Hz), 1 H), 8.31 (d, J = 8.4 Hz, 1 H), 8.38 (dd, J = 4.9, 1.0 Hz, 1 H), 9.18 (s, 1 H) | 674 |
| 38 | 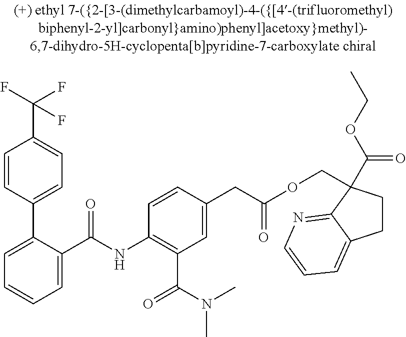 [SMILES: CCOC(=O)C1(COC(=O)Cc2ccc(NC(=O)c3ccccc3-c3ccc(C(F)(F)F)cc3)c(C(=O)N(C)C)c2)CCc2cccnc21] | δ 1.18 (t, 3 H), 2.03-2.15 (m, 1 H), 2.63 (ddd, J = 13.4, 8.9, 4.6 Hz, 1 H), 2.74-3.00 (m, 7 H), 3.00-3.12 (m, 1 H), 3.49 (s, 2 H), 4.10-4.19 (m, 2 H), 4.52 (d, J = 10.9 Hz, 1 H), 4.77 (d, J = 11.1 Hz, 1 H), 7.02 (d, J = 2.0 Hz, 1 H), 7.08- 7.15 (m, 2 H), 7.40 (dd, J = 7.7, 1.3 Hz, 1 H), 7.46- 7.58 (m, 3 H), 7.58-7.66 (m, 4 H), 7.70 (dd, J = 7.6, 1.6 Hz, 1 H), 8.31 (d, J = 8.4 Hz, 1 H), 8.38 (dd, J = 4.9, 1.0 Hz, 1 H), 9.18 (s, 1 H) | 674 |
| 39 | 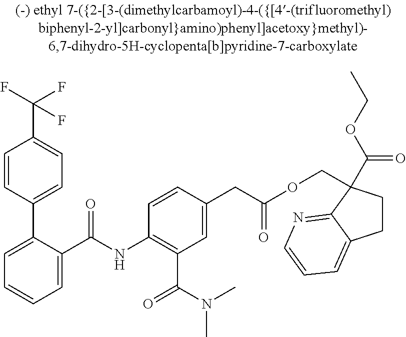 [SMILES: CCOC(=O)C1(COC(=O)Cc2ccc(NC(=O)c3ccccc3-c3ccc(C(F)(F)F)cc3)c(C(=O)N(C)C)c2)CCc2cccnc21] | δ 1.18 (t, 3 H), 2.03-2.15 (m, 1 H), 2.63 (ddd, J = 13.4, 8.9, 4.6 Hz, 1 H), 2.74-3.00 (m, 7 H), 3.00-3.12 (m, 1 H), 3.49 (s, 2 H), 4.10-4.19 (m, 2 H), 4.52 (d, J = 10.9 Hz, 1 H), 4.77 (d, J = 11.1 Hz, 1 H), 7.02 (d, J = 2.0 Hz, 1 H), 7.08- 7.15 (m, 2 H), 7.40 (dd, J = 7.7, 1.3 Hz, 1 H), 7.46- 7.58 (m, 3 H), 7.58-7.66 (m, 4 H), 7.70 (dd, J = 7.6, 1.6 Hz, 1 H), 8.31 (d, J = 8.4 Hz, 1 H), 8.38 (dd, J = 4.9, 1.0 Hz, 1 H), 9.18 (s, 1 H) | 674 |
| 40 | 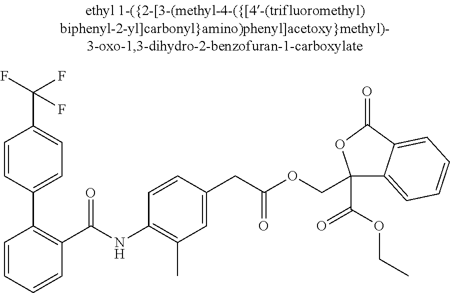 [SMILES: CCOC(=O)C1(COC(=O)Cc2ccc(NC(=O)c3ccccc3-c3ccc(C(F)(F)F)cc3)c(C)c2)OC(=O)c2ccccc21] | δ 1.23 (t, J = 7.1 Hz, 3 H), 1.72 (s, 3 H), 3.42 (s, 2 H), 4.17 (dq, J = 10.7, 7.1 Hz, 1 H), 4.25 (dq, J = 10.7, 7.1 Hz, 1 H), 4.68-4.79 (m, 2 H), 6.77-6.89 (m, 3 H), 7.45 (d, J = 7.3 Hz, 1 H), 7.51-7.63 (m, 4 H), 7.63-7.75 (m, 6 H), 7.83 (d, J = 7.7 Hz 2 H) | 632 |
| 41 | 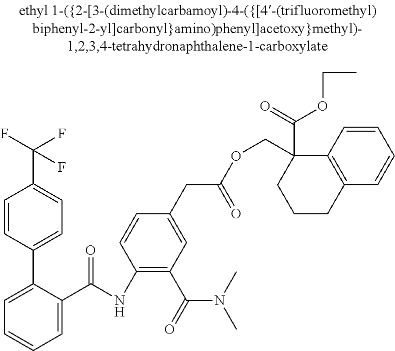 [SMILES: CCOC(=O)C1(COC(=O)Cc2ccc(NC(=O)c3ccccc3-c3ccc(C(F)(F)F)cc3)c(C(=O)N(C)C)c2)CCCc2ccccc21] | δ 1.18 (t, J = 7.1 Hz, 3 H), 1.84-1.94 (m, 2 H), 2.26-2.35 (m, 1 H), 2.75 (s, 2 H), 2.86 (br. s., 3 H), 2.95 (br. s., 3 H), 3.54 (s, 2 H), 4.12 (dq, J = 8.5, 7.1 Hz, 2 H), 4.32 (d, J = 11.1 Hz, 1 H), 4.57 (d, J = 11.1 Hz, 1 H), 7.06 (d, J = 1.9 Hz, 1 H), 7.08-7.13 (m, 2 H), 7.14-7.17 (m, 1 H), 7.20 (dd, J = 8.9, 2.5 Hz, 1 H), 7.24-7.27 (m, 2 H), 7.41 (dd, J = 7.5, 1.0 Hz, 1 H), 7.47- 7.57 (m, J = 14.6, 6.7, 6.7, 6.6 Hz, 2 H), 7.62 (s, 4 H), 7.71 (dt, J = 7.4, 0.8 Hz, 1 H), 8.36 (d, J = 8.5 Hz, 1 H), 9.17 (s, 1 H) | 687 |
| 42 | 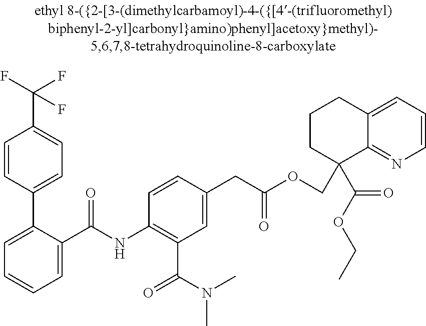 [SMILES: CCOC(=O)C1(COC(=O)Cc2ccc(NC(=O)c3ccccc3-c3ccc(C(F)(F)F)cc3)c(C(=O)N(C)C)c2)CCCc2cccnc21] | δ 1.16 (t, J = 7.1 Hz, 3 H), 1.64-1.80 (m, 1 H), 1.82-2.00 (m, 2 H), 2.17-2.26 (m, 1 H), 2.56-2.68 (m, 1 H), 2.72-3.02 (m, 7 H), 3.41-3.51 (m, 2 H), 4.06-4.20 (m, 2 H), 4.69 (s, 2 H), 6.99 (d, J = 2.0 Hz, 1 H), 7.03-7.09 (m, 2 H), 7.35-7.44 (m, 2 H), 7.46-7.58 (m, 2 H), 7.59-7.66 (m, 4 H), 7.71 (dd, J = 7.5, 1.5 Hz, 1 H), 8.30 (d, J = 8.6 Hz, 1 H), 8.35 (dd, J = 4.7, 1.8 Hz, 1 H), 9.18 (s, 1 H) | 688 |
| 43 | 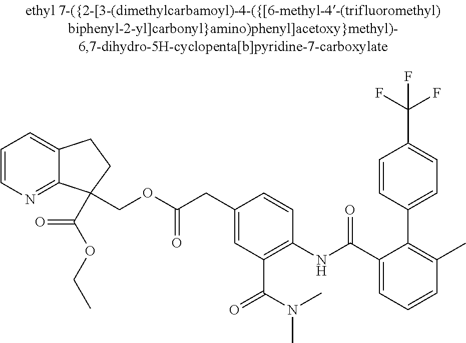 [SMILES: CCOC(=O)[C@]1(COC(=O)Cc2ccc(NC(=O)c3cccc(C)c3-c3ccc(C(F)(F)F)cc3)c(C(=O)N(C)C)c2)CCc2cccnc21] | (400 MHz, CD₃OD) δ 1.11 (t, J = 7.13 Hz, 3 H), 2.13 (s, 3H), 2.12-2.20 (m, 1H), 2.54 (ddd, J = 13.58, 8.99, 4.79 Hz, 1 H) 2.73-2.83 (m, 1 H), 2.84 (br. s., 3H), 2.92-3.00 (m, 1 H), 3.01 (br. s., 3 H), 3.52 (s, 2 H), 4.01-4.12 (m, 2 H), 4.51 (d, J = 10.9 Hz, 1 H), 4.64 (d, J = 10.9 Hz, 1 H), 6.98-7.03 (m, 1 H), 7.05-7.11 (m, 2 H) 7.14 (dd, J = 7.72, 4.98 Hz, 1 H) 7.35-7.47 (m, 3 H) 7.50 (d, J = 8.01 Hz, 2 H) 7.57 (dd, J = 7.62, 1.37 Hz, 1 H) 7.71 (d, J = 8.01 Hz, 2 H) 8.22 (dd, J = 4.69, 1.17 Hz, 1 H) | 688 |
| 44 | 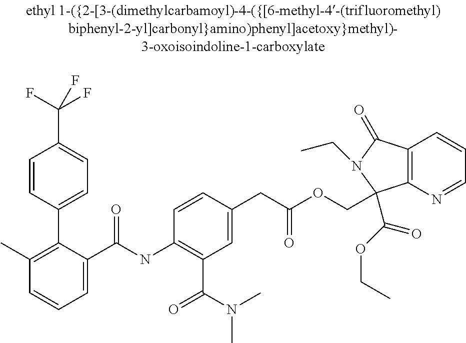 [SMILES: CCOC(=O)C1(COC(=O)Cc2ccc(NC(=O)c3cccc(C)c3-c3ccc(C(F)(F)F)cc3)c(C(=O)N(C)C)c2)c2ncccc2C(=O)N1CC] | δ 1.16 (t, J = 7.13 Hz, 3 H), 1.21 (t, J = 7.23 Hz, 3 H), 2.13 (s, 3 H), 2.90 (br. s., 3 H), 3.09 (br. s., 3 H), 3.24-3.37 (m, 2 H), 3.39-3.51 (m, 1 H), 3.52-3.64 (m, 1 H), 4.08- 4.25 (m, 2 H), 4.87 (d, J = 12.1 Hz ,1 H), 4.96 (d, J = 11.9 Hz, 1 H), 6.83-6.87 (m, 1 H), 6.88 (s, 1 H), 7.33 (dd, J = 7.62, 4.89 Hz, 1 H), 7.37-7.41 (m, 2 H), 7.43- 7.52 (m, 3 H), 7.62 (d, J = 8.01 Hz, 2 H), 7.90-8.11 (m, 2 H), 8.59 (dd,J = 4.89, 1.37 Hz, 1 H), 9.09 (s, 1 H) | 731 |

Similarly, examples 45 and 46 were prepared using analogous starting materials and preparations as described in example 1, and analyzed by chiral SFC using the following parameters: OJ-H 4.6×250 mm column; 90/10 CO₂/MeOH eluent; 2.5 mL/min. flow rate.

### Example 45

### (−)-Ethyl 1-({2-[3-(Dimethylcarbamoyl)-4-({[4′-(trifluoromethyl)biphenyl-2-yl]carbonyl}amino)phenyl]acetoxy}methyl)-1,2,3,4-tetrahydronaphthalene-1-carboxylate

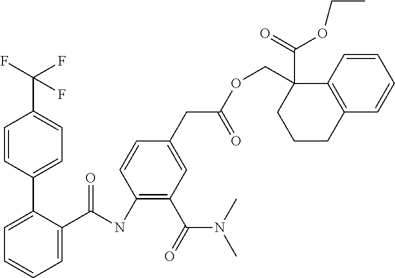 [SMILES: CCOC(=O)C1(COC(=O)Cc2ccc(NC(=O)c3ccccc3-c3ccc(C(F)(F)F)cc3)c(C(=O)N(C)C)c2)CCCc2ccccc21]

Chiral SFC t_{R}=4.25 min. LCMS (ESI) m/z: 687 [M+H].

### Example 46

### (+)-Ethyl 1-({2-[3-(Dimethylcarbamoyl)-4-({[4′-(trifluoromethyl)biphenyl-2-yl]carbonyl}amino)phenyl]acetoxy}methyl)-1,2,3,4-tetrahydronaphthalene-1-carboxylate

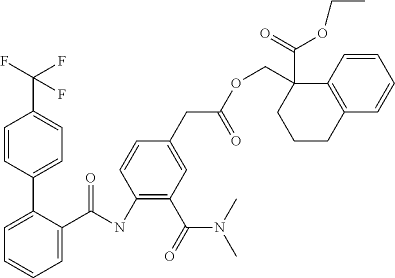 [SMILES: CCOC(=O)C1(COC(=O)Cc2ccc(NC(=O)c3ccccc3-c3ccc(C(F)(F)F)cc3)c(C(=O)N(C)C)c2)CCCc2ccccc21]

Chiral SFC t_{R}=4.29 min. LCMS (ESI) m/z: 687 [M+H].

Similarly, examples 47 and 48 were prepared using analogous starting materials and preparations as described in Example 1, and analyzed by chiral SFC using the following parameters: Chiralpak AD-H 10×250 mm column; 75/25 CO₂/EtOH eluent modified with 0.2% isopropylamine; 10 mL/min. flow rate.

### Example 47

### Ethyl 7-({2-[3-(Dimethylcarbamoyl)-4-({[6-methyl-4′-(trifluoromethyl)biphenyl-2-yl]carbonyl}amino)phenyl]acetoxy}methyl)-6-ethyl-5-oxo-6,7-dihydro-5H-pyrrolo[3,4-b]pyridine-7-carboxylate

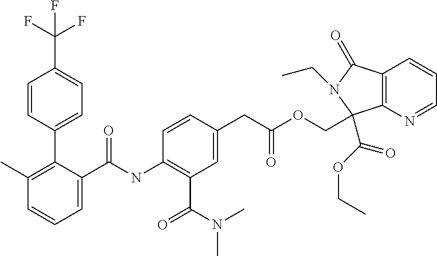 [SMILES: CCOC(=O)C1(COC(=O)Cc2ccc(NC(=O)c3cccc(C)c3-c3ccc(C(F)(F)F)cc3)c(C(=O)N(C)C)c2)c2ncccc2C(=O)N1CC]

Chiral SFC t_{R}=4.38 min. LCMS (ESI) m/z: 731 [M+H].

### Example 48

### Ethyl 7-({2-[3-(Dimethylcarbamoyl)-4-({[6-methyl-4′-(trifluoromethyl)biphenyl-2-yl]carbonyl}amino)phenyl]acetoxy}methyl)-6-ethyl-5-oxo-6,7-dihydro-5H-pyrrolo[3,4-b]pyridine-7-carboxylate

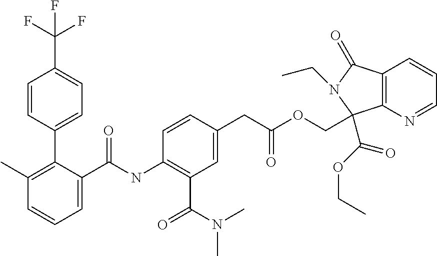 [SMILES: CCOC(=O)C1(COC(=O)Cc2ccc(NC(=O)c3cccc(C)c3-c3ccc(C(F)(F)F)cc3)c(C(=O)N(C)C)c2)c2ncccc2C(=O)N1CC]

Chiral SFC t_{R}=6.77 min. LCMS (ESI) m/z: 731 [M+H].

### Example 49

### Ethyl (1R)-1-({2-[3-(Dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate

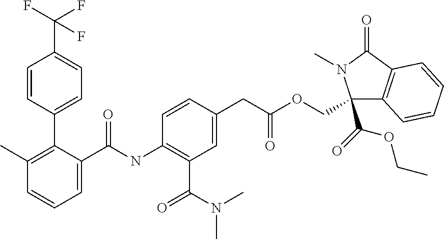 [SMILES: CCOC(=O)[C@]1(COC(=O)Cc2ccc(NC(=O)c3cccc(C)c3-c3ccc(C(F)(F)F)cc3)c(C(=O)N(C)C)c2)c2ccccc2C(=O)N1C]

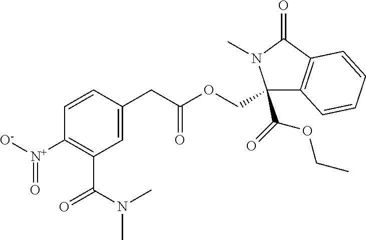 [SMILES: CCOC(=O)[C@]1(COC(=O)Cc2ccc([N+](=O)[O-])c(C(=O)N(C)C)c2)c2ccccc2C(=O)N1C]

### Step A: Ethyl (1R)-1-({2-[3-(Dimethylcarbamoyl)-4-nitrophenyl]acetoxy}methyl)-2-methyl-3-oxoisoindoline-1-carboxylate

To a solution of ethyl (1R)-1-(hydroxymethyl)-2-methyl-3-oxoisoindoline-1-carboxylate (2.04 g, 8.19 mmol) in CH₂Cl₂ (40. mL) was added [3-(dimethylcarbamoyl)-4-nitrophenyl]acetic acid (2.89 g, 11.5 mmol), 1-ethyl-3-(3-dimethylaminopropyl)carbodiimide hydrogen chloride salt (2.36 g, 12.3 mmol), and DMAP (1.50 g, 12.3 mmol). The reaction mixture was stirred at room temperature for 18 hours, after which the solvent was removed by rotary evaporation. The residue was partitioned between EtOAc and sat. aq. NH₄Cl. The organic layer was washed with sat. aq. NaHCO₃ and brine, dried over Na₂SO₄, and concentrated to dryness. The residue was purified by MPLC (gradient from pure heptane to pure EtOAc) to afford the title compound (3.36 g, 84.8% yield). LCMS (ESI) m/z: 484.2 [M+H] (100%). ¹H NMR (400 MHz, CDCl₃) δ 1.22 (t, J=7.1 Hz, 3H), 2.82 (s, 3H), 3.09 (s, 3H), 3.16 (s, 3H), 3.46-3.58 (m, 2H), 4.09-4.20 (m, 1H), 4.21-4.31 (m, 1H), 4.78 (d, J=11.9 Hz, 1H), 4.88 (d, J=11.9 Hz, 1H), 7.13 (s, 1H), 7.10 (s, 1H), 7.49-7.59 (m, 3H), 7.79-7.85 (m, 1H), 8.04 (d, J=8.6 Hz, 1H).

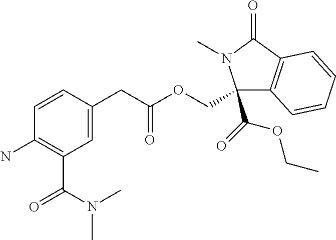 [SMILES: CCOC(=O)[C@]1(COC(=O)Cc2ccc(N)c(C(=O)N(C)C)c2)c2ccccc2C(=O)N1C]

### Step B: Ethyl (1R)-1-({2-[4-Amino-3-(dimethylcarbamoyl)phenyl]acetoxy}methyl)-2-methyl-3-oxoisoindoline-1-carboxylate

To a solution of ethyl (1R)-1-({2-[3-(dimethylcarbamoyl)-4-nitrophenyl]acetoxy}methyl)-2-methyl-3-oxoisoindoline-1-carboxylate (3.36 g, 6.95 mmol) in EtOH (23 mL) was added iron powder (1.16 g, 20.8 mmol) and glacial acetic acid (3.98 mL, 69.5 mmol). The reaction mixture was refluxed for 1 hour, cooled to room temperature, and then partitioned between CH₂Cl₂ and sat. aq. NaHCO₃. The aq. layer was extracted twice with CH₂Cl₂. The combined organic layers were dried over Na₂SO₄ and subsequently concentrated to dryness to afford the title compound (2.90 g, 92% yield). LCMS (ESI) m/z: 454.3 [M+H] (100%). ¹H NMR (400 MHz, CDCl₃) δ 1.22 (t, J=7.1 Hz, 3H), 3.04 (br. s., 6H), 3.09 (s, 3H), 3.28-3.39 (m, 2H), 4.12 (dq, J=10.7, 7.1 Hz, 1H), 4.24 (dq, J=10.7, 7.1 Hz, 1H), 4.32 (br. s., 2H), 4.62 (d, J=11.9 Hz, 1H), 4.85 (d, J=11.9 Hz, 1H), 6.61 (d, J=8.4 Hz, 1H), 6.79-6.86 (m, 2H), 7.48-7.58 (m, 3H), 7.81-7.87 (m, 1H).

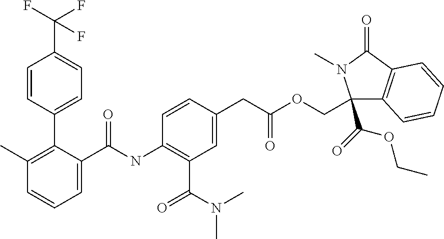 [SMILES: CCOC(=O)[C@]1(COC(=O)Cc2ccc(NC(=O)c3cccc(C)c3-c3ccc(C(F)(F)F)cc3)c(C(=O)N(C)C)c2)c2ccccc2C(=O)N1C]

### Step C: Ethyl (1R)-1-({2-[3-(Dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate

To a solution of 6-methyl-4′-(trifluoromethyl)biphenyl-2-carboxylic acid (2 g, 7.13 mmol) in CH₂Cl₂ (34 mL) was added oxalyl chloride (0.907 mL, 10.2 mmol) and catalytic amount of DMF (0.150 mL, 2.04 mmol). The resulting light yellow solution was stirred at room temperature for 1 hour and then concentrated by rotary evaporation. The residue was dissolved in CH₂Cl₂ (34 mL), and ethyl (1R)-1-({2-[4-amino-3-(dimethylcarbamoyl)phenyl]acetoxy}methyl)-2-methyl-3-oxoisoindoline-1-carboxylate (3.09 g, 6.80 mmol) and DIEA (2.48 mL, 14.3 mmol) were added. The reaction mixture was stirred at room temperature for 1 hour, quenched with sat. aq. NH₄Cl, and then extracted with CH₂Cl₂. The aq. layer was isolated and extracted twice with additional CH₂Cl₂. The combined organic layers were washed with brine, dried over Na₂SO₄, and concentrated to afford an oil. This oil was purified by MPLC (gradient from 4:21 EtOAc/heptane to pure EtOAc) to afford the title compound (4.72 g, 97% yield). LCMS (ESI) m/z: 716.4 [M+H] (100%). ¹H NMR (400 MHz, CDCl₃) δ 1.19 (t, J=7.1 Hz, 3H), 2.12 (s, 3H), 2.90 (br. s., 3H), 3.08 (s, 3H), 3.11 (br. s., 3H), 3.30-3.41 (m, 2H), 4.11 (dq, J=10.7, 7.1 Hz, 1H), 4.22 (dq, J=10.7, 7.1 Hz, 1H), 4.67 (d, J=11.9 Hz, 1H), 4.81 (d, J=11.9 Hz, 1H), 6.90-6.97 (m, 2H), 7.34-7.40 (m, 2H), 7.42-7.51 (m, 6H), 7.61 (d, J=8.0 Hz, 2H), 7.79-7.83 (m, 1H), 8.06 (d, J=8.4 Hz, 1H), 9.07 (s, 1H).

### Example 50

### Ethyl (1R)-1-({2-[3-(dimethylcarbamoyl)-4-{[(4′-isopropoxybiphenyl-2-yl)carbonyl]amino}phenyl]acetoxy}methyl)-2-methyl-3-oxoisoindoline-1-carboxylate

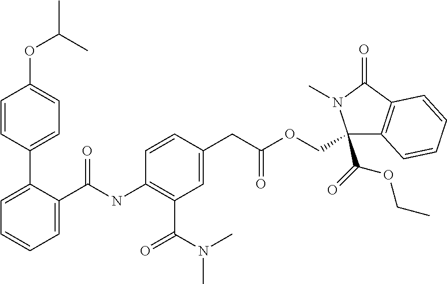 [SMILES: CCOC(=O)[C@]1(COC(=O)Cc2ccc(NC(=O)c3ccccc3-c3ccc(OC(C)C)cc3)c(C(=O)N(C)C)c2)c2ccccc2C(=O)N1C]

To a solution of 4′-isopropoxybiphenyl-2-carboxylic acid (53.3 mg, 0.208 mmol) in CH₂Cl₂ (1.0 mL) was added oxalyl chloride (0.148 mL, 0.297 mmol) and catalytic amount of DMF (4 μL, 0.06 mmol). The resulting light yellow solution was stirred at room temperature for 1 hour and then concentrated by rotary evaporation. The residue was dissolved in CH₂Cl₂ (1.0 mL), and ethyl (1R)-1-({2-[4-amino-3-(dimethylcarbamoyl)phenyl]acetoxy}methyl)-2-methyl-3-oxoisoindoline-1-carboxylate (89.8 mg, 0.198 mmol) and DIEA (73 μL, 0.42 mmol) were added. The reaction mixture was stirred at room temperature for 1 hour, quenched with sat. aq. NH₄Cl, and then extracted with CH₂Cl₂. The aq. layer was isolated and extracted twice with additional CH₂Cl₂. The combined organic layers were washed with brine, dried over Na₂SO₄, and concentrated to afford an oil. This oil was purified by MPLC (gradient from 4:21 EtOAc/heptane to 4:1 EtOAc/heptane) to afford the title compound (131.8 mg, 96 yield). LCMS (ESI) m/z: 692 [M+H] (100%). ¹H NMR (400 MHz, CDCl₃) δ 1.20 (t, J=7.12 Hz, 3H), 1.31 (d, J=6.05 Hz, 6H), 2.82 (br. s., 3H), 2.95 (br. s., 3H), 3.09 (s, 3H), 3.30-3.44 (m, 2H), 4.05-4.17 (m, 1H), 4.23 (dq, J=10.83, 7.19 Hz, 1H), 4.52 (dt, J=12.10, 6.05 Hz, 1H), 4.67 (d, J=11.90 Hz, 1H), 4.82 (d, J=11.90 Hz, 1H), 6.87 (d, J=8.39 Hz, 3H), 7.01 (dd, J=8.49, 1.66 Hz, 1H), 7.36-7.42 (m, 4H), 7.43-7.55 (m, 4H), 7.63-7.70 (m, 1H), 7.82 (dd, J=4.88, 2.93 Hz, 1H), 8.31 (d, J=8.39 Hz, 1H), 8.73 (s, 1H).

### Examples 51-65

The following compounds were prepared following the general procedure for Example 49 using analogous starting materials. The appropriate acid, core and alcohol are substituted and described in the preparations section, are commercially available, or may be prepared by one skilled in the art.

| Ex. | Structure and | ¹H NMR | LCMS |
|---|---|---|---|
| # | Name | (400 MHz, CDCl₃) | (ESI) m/z |
| 51 | ethyl 1-({2-[3- (dimethylcarbamoyl)-4-{[(4′- isopropoxy-6-methylbiphenyl-2- yl)carbonyl]amino}phenyl] acetoxy}methyl)-2-methyl-3- oxoisoindoline-1-carboxylate | δ 1.20 (t, J = 7.1 Hz, 3 H), 1.28 (d, J = 6.0 Hz, 6 H), 2.18 (s, 3 H), 2.88 (br. s., 3 H), 3.01-3.13 (m, 3 H), 3.09 (s, 3 H), 3.30- 3.41 (m, 2 H), 4.11 (dq, J = 10.7, 7.1 Hz, 1 H), 4.23 (dq, J = 10.7, 7.2 Hz, 1 H), 4.50 (dt, J = 12.1, 6.1 Hz, 1 H), 4.67 (d, J = 11.9 Hz, 1 H), 4.81 (d, J = 11.9 Hz, 1 H), 6.83-6.90 (m, 3 H), 6.94 (dd, J = 8.5, 2.0 Hz, 1 H), 7.18- 7.24 (m, 2 H), 7.28-7.38 (m, 2 H), 7.41-7.54 (m, 4 H), 7.78- 7.83 (m, 1 H), 8.05 (d, J = 8.4 Hz, 1 H), 8.61 (s, 1 H). | 706 |
| | 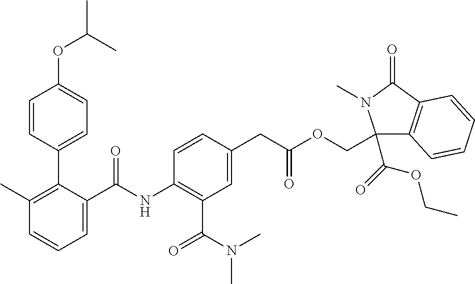 [SMILES: CCOC(=O)C1(COC(=O)Cc2ccc(NC(=O)c3cccc(C)c3-c3ccc(OC(C)C)cc3)c(C(=O)N(C)C)c2)c2ccccc2C(=O)N1C] | | |
| 53 | ethyl 1-({2-[3- (dimethylcarbamoyl)-4-{[(4′- isopropoxy-6-methylbiphenyl-2- yl)carbonyl]amino}phenyl] acetoxy}methyl)-2-methyl-3- oxoisoindoline-1-carboxylate | δ 1.15-1.23 (m, 9 H), 2.15 (s, 3 H), 2.87 (quin, J = 6.9 Hz, 4 H), 3.02-3.13 (m, 6 H), 3.29-3.40 (m, 2 H), 4.06-4.17 (m, 1 H), 4.17-4.29 (m, 1 H), 4.66 (d, J = 11.9 Hz, 1 H), 4.81 (d, J = 11.9 Hz, 1 H), 6.87-6.94 (m, 2 H), 7.17-7.25 (m, 4 H), 7.28- 7.37 (m, 2 H), 7.40-7.58 (m, 4 H), 7.77-7.84 (m, 2 H), 8.53 (s, 1 H). | 690 |
| | 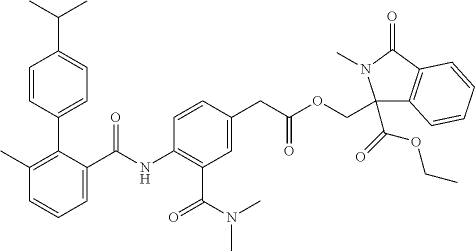 [SMILES: CCOC(=O)C1(COC(=O)Cc2ccc(NC(=O)c3cccc(C)c3-c3ccc(C(C)C)cc3)c(C(=O)N(C)C)c2)c2ccccc2C(=O)N1C] | | |
| 54 | ethyl 1-({2-[3- (dimethylcarbamoyl)-4-{[(4′- isopropoxybiphenyl-2- yl)carbonyl]amino}phenyl] acetoxy}methyl)-2-methyl-3- oxoisoindoline-1-carboxylate | δ 1.20 (t, J = 7.2 Hz, 3 H), 1.32 (d, J = 6.2 Hz, 6 H), 2.82 (br. s., 3 H), 2.88-3.00 (m, 3 H), 3.09 (s, 3 H), 3.31-3.43 (m, 2 H), 4.11 (dq, J = 10.8, 7.1 Hz, 1 H), 4.23 (dq, J = 10.8, 7.1 Hz, 1 H), 4.53 (quin, J = 6.1 Hz, 1 H), 4.67 (d, J = 11.7Hz, 1 H), 4.82 (d, J = 11.9 Hz, 1 H), 6.84-6.90 (m, 3 H), 7.01 (dd, J = 8.5, 2.0 Hz, 1 H), 7.36-7.43 (m, 4 H), 7.45- 7.54 (m, 4 H), 7.64-7.69 (m, 1 H), 7.79-7.85 (m, 1 H), 8.31 (d, J = 8.6 Hz, 1 H), 8.73 (s, 1 H). | 692 |
| | 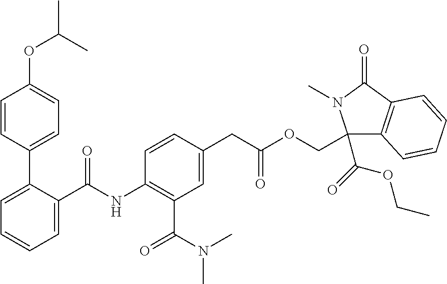 [SMILES: CCOC(=O)C1(COC(=O)Cc2ccc(NC(=O)c3ccccc3-c3ccc(OC(C)C)cc3)c(C(=O)N(C)C)c2)c2ccccc2C(=O)N1C] | | |
| 55 | ethyl l-({2-[4-({[4′,5- bis(trifluoromethyl)biphenyl-2- yl]carbonyl}amino)-3- (dimethylcarbamoyl)phenyl] acetoxy}methyl)-2-methyl-3- oxoisoindoline-1-carboxylate | δ 1.21 (t, J = 7.1 Hz, 3 H), 2.90 (br. s., 3 H), 2.97 (br. s., 3 H), 3.08 (s, 3 H), 3.32-3.43 (m, 2 H), 4.07-4.17 (m, 1 H), 4.19- 4.29 (m, 1 H), 4.70 (d, J = 11.9 Hz, 1 H), 4.83 (d, J = 11.9 Hz, 1 H), 6.93 (d, J = 2.0 Hz, 1 H), 7.02 (dd, J = 8.6, 2.1 Hz, 1 H), 7.47-7.55 (m, 3 H), 7.61-7.70 (m, 5 H), 7.74-7.79 (m, 1 H), 7.80-7.86 (m, 2 H), 8.28 (d, J = 8.4 Hz, 1 H), 9.36 (s, 1 H). | 770 |
| | 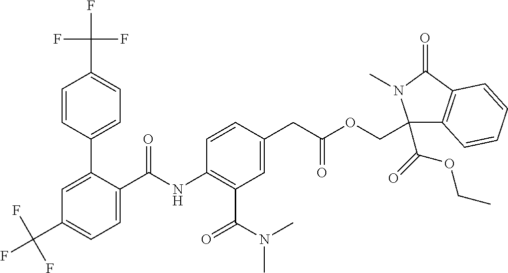 [SMILES: CCOC(=O)C1(COC(=O)Cc2ccc(NC(=O)c3ccc(C(F)(F)F)cc3-c3ccc(C(F)(F)F)cc3)c(C(=O)N(C)C)c2)c2ccccc2C(=O)N1C] | | |
| 56 | ethyl 1-({2-[3- (dimethylcarbamoyl)-4-({[5- methyl-4′- (trifluoromethyl)biphenyl-2- yl]carbonyl}amino)phenyl] acetoxy}methyl)-2-methyl-3- oxoisoindoline-1-carboxylate | δ 1.20 (t, J = 7.1 Hz, 3 H), 2.45 (s, 3 H), 2.84 (br. s., 3 H), 2.96 (br. s., 3 H), 3.08 (s, 3 H), 3.32- 3.42 (m, 2 H), 4.12 (dq, J = 10.8, 7.1 Hz, 1 H), 4.23 (dq, J = 10.8, 7.1 Hz, 1 H), 4.68 (d, J = 11.9 Hz, 1 H), 4.82 (d, J = 11.9 Hz, 1 H), 6.90 (d, J = 2.1 Hz, 1 H), 7.20 (s, 1 H), 7.28-7.32 (m, 1 H), 7.46-7.55 (m, 3 H), 7.56- 7.65 (m, 5 H), 7.79-7.85 (m, 1 H), 8.30 (d, J = 8.4 Hz, 1 H), 9.11 (s, 1 H). | 716 |
| | 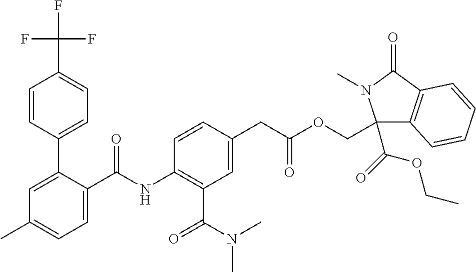 [SMILES: CCOC(=O)C1(COC(=O)Cc2ccc(NC(=O)c3ccc(C)cc3-c3ccc(C(F)(F)F)cc3)c(C(=O)N(C)C)c2)c2ccccc2C(=O)N1C] | | |
| 57 | ethyl 7-({2-[3- (dimethylcarbamoyl)-4-{[(4′- isopropoxy-6-methylbiphenyl-2- yl)carbonyl]amino}phenyl] acetoxy}methyl)-6,7-dihydro-5H- cyclopenta[b]pyridine-7- carboxylate | δ 1.18 (t, J = 7.1 Hz, 3 H), 1.28 (d, J = 5.5 Hz, 6 H), 2.04-2.16 (m, 1 H), 2.18 (s, 3 H), 2.62 (ddd, J = 13.4, 8.9, 4.6 Hz, 1 H), 2.73-2.84 (m, 1 H), 2.89 (br. s., 3H), 2.98-3.10 (m, 4 H), 3.46 (s, 2H), 4.15 (q, J = 7.0 Hz, 2 H), 4.44-4.53 (m, 1 H), 4.53 (d, J = 11.1 Hz, 1 H), 4.75 (d, J = 10.9 Hz, 1 H), 6.82-6.88 (m, 2 H), 6.97 (d, J = 2.1 Hz, 1 H), 6.99-7.05 (m, 1 H), 7.06-7.12 (m, 1 H), 7.19-7.24 (m, 2 H), 7.28-7.36 (m, 2 H), 7.41-7.53 (m, 2 H), 8.08 (d, J = 8.4 Hz, 1 H), 8.37 (dd, J = 4.9, 1.6 Hz, 1 H), 8.65 (s, 1 H). | 678 |
| | 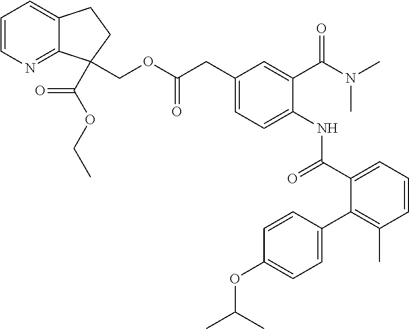 [SMILES: CCOC(=O)C1(COC(=O)Cc2ccc(NC(=O)c3cccc(C)c3-c3ccc(OC(C)C)cc3)c(C(=O)N(C)C)c2)CCc2cccnc21] | | |
| 58 | ethyl 7-({2-[4-({[4′,5- bis(trifluoromethyl)biphenyl-2- yl]carbonyl}amino)-3- (dimethylcarbamoyl)phenyl] acetoxy}methyl)-6,7-dihydro-5H- cyclopenta[b]pyridine-7- carboxylate | δ 1.18 (t, J = 7.1 Hz, 3 H), 2.07- 2.19 (m, 1 H), 2.64 (ddd, J = 13.4, 8.9,4.7 Hz, 1 H), 2.78- 3.00 (m, 7 H), 3.01-3.12 (m, 1 H), 3.50 (s, 2 H), 4.15 (q, J = 7.2 Hz, 2 H), 4.52 (d, J = 10.9 Hz, 1 H), 4.78 (d, J = 11.1 Hz, 1 H), 7.05 (d, J = 2.1 Hz, 1 H), 7.09- 7.19 (m, 2 H), 7.46-7.57 (m, 1 H), 7.61-7.70 (m, 5 H), 7.74- 7.79 (m, 1 H), 7.80-7.85 (m, 1 H), 8.29 (d, J = 8.6 Hz, 1 H), 8.39 (dd, J = 5.0, 1.5 Hz, 1 H), 9.38 (s, 1 H). | 742 |
| | 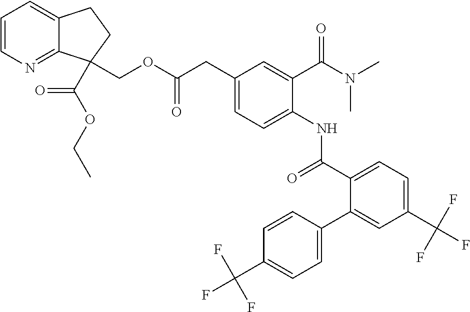 [SMILES: CCOC(=O)C1(COC(=O)Cc2ccc(NC(=O)c3ccc(C(F)(F)F)cc3-c3ccc(C(F)(F)F)cc3)c(C(=O)N(C)C)c2)CCc2cccnc21] | | |
| 59 | ethyl 7-({2-[3- (dimethylcarbamoyl)-4-({[5- methyl-4′- (trifluoromethyl)biphenyl-2- yl]carbonyl}amino)phenyl] acetoxy}methyl)-6,7-dihydro-5H- cyclopenta[b]pyridine-7- carboxylate | δ 1.18 (t, 3 H), 2.10 (ddd, J = 13.3, 9.1, 6.8 Hz, 1 H), 2.45 (s, 3H), 2.63 (ddd, J = 13.4, 8.9, 4.6 Hz, 1 H), 2.76-3.00 (m, 7 H), 3.00-3.12 (m, 1 H), 3.48 (s, 2H), 4.11-4.19 (m, 2 H), 4.52 (d, J = 10.9 Hz, 1 H), 4.77 (d, J = 10.9 Hz, 1 H), 7.01 (d, J = 2.1 Hz, 1 H), 7.07-7.14 (m, 2 H), 7.20 (s, 1 H), 7.30 (dd, J = 7.8, 1.0 Hz, 1 H), 7.52 (dd, J = 7.6, 1.6 Hz, 1 H), 7.56-7.65 (m, 5 H), 8.31 (d, J = 8.4 Hz, 1 H), 8.38 (dd, J = 4.9, 1.6 Hz, 1 H), 9.14 (s, 1 H). | 688 |
| | 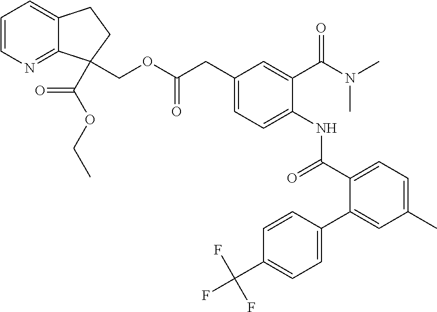 [SMILES: CCOC(=O)C1(COC(=O)Cc2ccc(NC(=O)c3ccc(C)cc3-c3ccc(C(F)(F)F)cc3)c(C(=O)N(C)C)c2)CCc2cccnc21] | | |
| 60 | ethyl 7-({2-[3- (dimethylcarbamoyl)-4-({[4′- isopropoxybiphenyl-2- yl)carbonyl]amino}phenyl] acetoxy}methyl)-6,7-dihydro-5H- cyclopenta[b]pyridine-7- carboxylate | δ 1.19 (t, J = 7.1 Hz, 3 H), 1.31 (dd, J = 6.1, 1.37 Hz, 6 H), 2.11 (ddd, J = 13.3, 9.14, 6.84 Hz, 1 H), 2.63 (ddd, J = 13.4, 8.89, 4.69 Hz, 1 H), 2.74-2.98 (m, 7 H), 3.02-3.10 (m, 1 H), 3.48 (s, 2 H), 4.11-4.20 (m, 2 H), 4.49- 4.53 (m, 1 H), 4.54 (d, J = 11.1 Hz, 1H), 4.77 (d, J = 11.1 Hz, 1 H), 6.87 (d, J = 8.8 Hz, 2 H), 6.96 (d, J = 2.2 Hz, 1 H), 7.07- 7.15 (m, 2 H), 7.35-7.43 (m, 4 H), 7.46-7.56 (m, 2 H), 7.67 (dd, J = 7.9, 1.27 Hz, 1 H), 8.33 (d, J = 8.4 Hz, 1 H), 8.38 (dd, J = 4.5, 1.17 Hz, 1 H), 8.77 (s, 1 H) | 664 |
| | 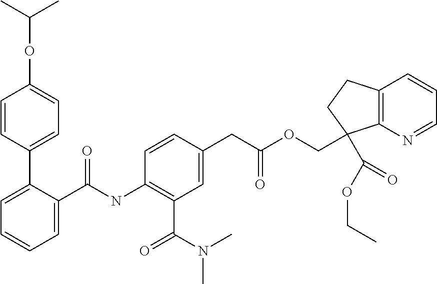 [SMILES: CCOC(=O)C1(COC(=O)Cc2ccc(NC(=O)c3ccccc3-c3ccc(OC(C)C)cc3)c(C(=O)N(C)C)c2)CCc2cccnc21] | | |
| 61 | ethyl 7-({2-[3- (dimethylcarbamoyl)-4-({[6- methoxy-4′- (trifluoromethyl)biphenyl-2- yl]carbonyl}amino)phenyl] acetoxy}methyl)-6,7-dihydro-5H- cyclopenta[b]pyridine-7- carboxylate | δ 1.18 (t, J = 7.12 Hz, 3 H), 2.05- 2.13 (m, 1 H), 2.62 (ddd, J = 13.41, 8.93, 4.58 Hz, 1 H), 2.74-3.11 (m, 8H), 3.47 (s, 2 H), 3.79 (s, 3 H), 4.15 (q, J = 7.22 Hz, 2 H), 4.52 (d, J = 11.12 Hz, 1 H), 4.76 (d, J = 11.12 Hz, 1 H), 7.01 (d, J = 2.15 Hz, 1 H), 7.04-7.12 (m, 3H), 7.23-7.27 (m, 2 H), 7.45 (t, J = 8.00 Hz, 1 H), 7.50-7.61 (m, 4 H), 8.17 (d, J = 8.58 Hz, 1 H), 8.35-8.39 (m, 1 H), 9.04 (s, 1 H) | 704 |
| | 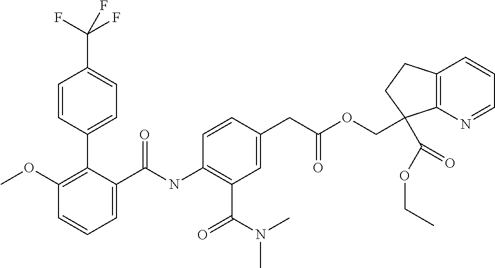 [SMILES: CCOC(=O)C1(COC(=O)Cc2ccc(NC(=O)c3cccc(OC)c3-c3ccc(C(F)(F)F)cc3)c(C(=O)N(C)C)c2)CCc2cccnc21] | | |
| 62 | ethyl (1R)-2-methyl-1-({2-[4-({[6- methyl-4′- (trifluoromethyl)biphenyl-2- yl]carbonyl}amino)-3-(piperidin-1- ylcarbonyl)phenyl]acetoxy}methyl)- 3-oxoisoindoline-1-carboxylate | δ 1.19 (t, J = 7.2 Hz, 3 H), 1.45- 1.79 (m, 6 H), 2.12 (s, 3 H), 3.09 (s, 3 H), 3.22-3.46 (m, 2 H), 3.31-3.42 (m, 2 H), 3.48- 3.77 (m, 2 H), 4.06-4.16 (m, 1 H), 4.18-4.27 (m, 1 H), 4.65 (d, J = 11.8 Hz, 1 H), 4.83 (d, J = 11.9 Hz, 1 H), 6.92-6.96 (m, 2H), 7.34-7.52 (m, 8 H), 7.63 (d, J = 8.0 Hz, 2 H), 7.80-7.83 (m, 1 H), 7.95 (d, J = 8.6 Hz, 1 H), 8.95 (s, 1 H) | 756 |
| | 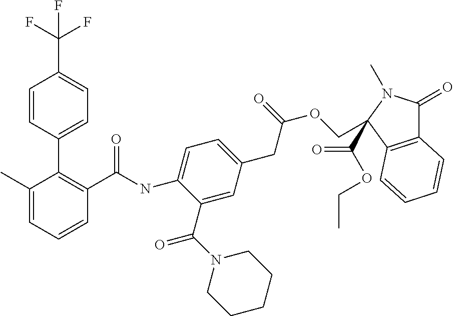 [SMILES: CCOC(=O)[C@]1(COC(=O)Cc2ccc(NC(=O)c3cccc(C)c3-c3ccc(C(F)(F)F)cc3)c(C(=O)N3CCCCC3)c2)c2ccccc2C(=O)N1C] | | |
| 63 | ethyl (1R)-1-({2-[3- (dimethylcarbamoyl)-4-{[(4′- isopropoxy-6-methoxybiphenyl-2- yl)carbonyl]amino}phenyl] acetoxy}methyl)-2-methyl-3- oxoisoindoline-1-carboxylate | δ 1.20 (t, J = 7.1 Hz, 3 H), 1.27 (s, 3 H), 1.29 (s, 3 H), 2.83 (br. s., 3 H), 3.03 (br. s., 3 H), 3.09 (s, 3 H), 3.28-3.41 (m, 2 H), 3.79 (s, 3 H), 4.04-4.16 (m, 1 H), 4.17-4.29 (m, 1 H), 4.49 (quin, J = 6.0 Hz, 1 H), 4.66 (d, J = 11.8 Hz, 1 H), 4.81 (d, J = 11.9 Hz, 1 H), 6.81-6.87 (m, 3 H), 6.96 (dd, J = 8.5, 2.1 Hz, 1 H), 7.03 (dd, J = 8.4, 1.0 Hz, 1 H), 7.23 (dd, J = 7.7, 1.1 Hz, 1 H), 7.31-7.39 (m, 3 H), 7.45- 7.54 (m, 3 H), 7.78-7.84 (m, 1 H), 8.17 (d, J = 8.6 Hz, 1 H), 8.58 (s, 1 H) | 722 |
| | 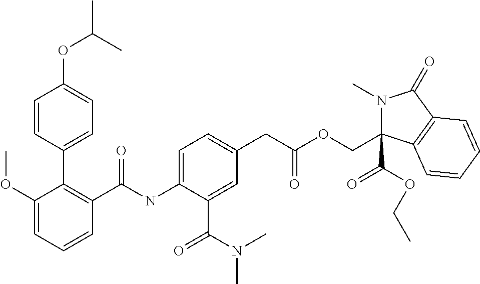 [SMILES: CCOC(=O)[C@]1(COC(=O)Cc2ccc(NC(=O)c3cccc(OC)c3-c3ccc(OC(C)C)cc3)c(C(=O)N(C)C)c2)c2ccccc2C(=O)N1C] | | |
| 64 | ethyl (1R)-1-({2-[3- (dimethylcarbamoyl)-4-{[(6- methoxybiphenyl-2- yl)carbonyl]amino}phenyl] acetoxy}methyl)-2-methyl-3- oxoisoindoline-1-carboxylate | δ 1.20 (t, J = 7.2 Hz, 3 H), 2.83 (br. s., 3 H), 2.99-3.06 (m, 3 H), 3.07 (s, 3 H), 3.30-3.41 (m, 2 H), 3.78 (s, 3 H), 4.05- 4.16 (m, 1 H), 4.17-4.28 (m, 1 H), 4.65 (d, J = 11.8 Hz, 1 H), 4.81 (d, J = 11.9 Hz, 1 H), 6.86 (d, J = 2.0 Hz, 1 H), 6.95 (dd, J = 8.5, 2.1 Hz, 1 H), 7.06 (dd, J = 8.3, 1.0 Hz, 1 H), 7.23-7.29 (m, 3 H), 7.31-7.45 (m, 5 H), 7.46-7.54 (m, 2 H), 7.78-7.83 (m, 1 H), 8.12 (d, J = 8.5 Hz, 1 H), 8.62 (s, 1 H) | 664 |
| | 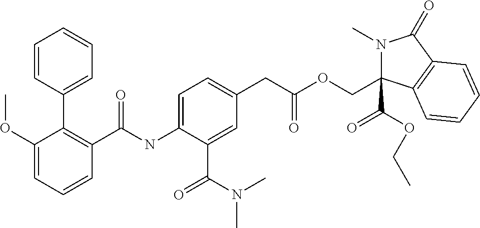 [SMILES: CCOC(=O)[C@]1(COC(=O)Cc2ccc(NC(=O)c3cccc(OC)c3-c3ccccc3)c(C(=O)N(C)C)c2)c2ccccc2C(=O)N1C] | | |
| 65 | methyl 1-({2-[3- (dimethylcarbamoyl)-4-({[6- methyl-4′- (trifluoromethyl)biphenyl-2- yl]carbonyl}amino)phenyl] acetoxy}methyl)-2-ethyl-3- oxoisoindoline-1-carboxylate | δ 1.23 (d, J = 7.22 Hz, 3 H), 2.12 (s, 3 H), 2.90 (br. s., 3 H), 3.09 (br. s., 3 H), 3.27-3.45 (m, 3 H), 3.67 (s, 3 H), 3.67-3.76 (m, 1 H), 4.62-4.80 (m, 2 H), 6.92-6.99 (m, 2 H), 7.35-7.52 (m, 8 H), 7.61 (d, J = 8.00 Hz, 2 H), 7.77-7.83 (m, 1 H), 8.07 (d, J = 8.39 Hz, 1 H), 9.07 (s, 1 H) | 716 |
| | 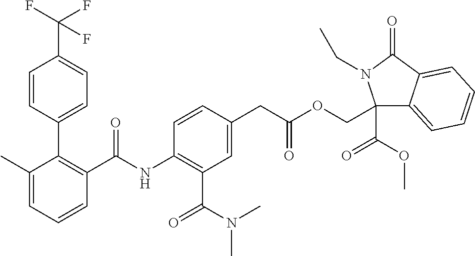 [SMILES: CCN1C(=O)c2ccccc2C1(COC(=O)Cc1ccc(NC(=O)c2cccc(C)c2-c2ccc(C(F)(F)F)cc2)c(C(=O)N(C)C)c1)C(=O)OC] | | |

### Examples 66-75

The following compounds were prepared using procedures analogous to example 49 using the appropriate staring materials and purified by preparative HPLC. HPLC analysis of the products was performed on a Waters Atlantis dC18 4.6×50 mm, 5 μm column using the following program: linear gradient from 5:95 MeCN/H₂O to 95:5 MeCN/H₂O over 4.0 min, hold at 95:5 MeCN/H₂O for 5 min. A 0.05% TFA modifier and a flow rate of 2.0 mL/min. were used.

| | | Retention | LCMS |
|---|---|---|---|
| Ex. | Structure and | time | (ESI) |
| # | Name | (min) | m/z |
| 66 | ethyl (1R)-1-({2-[4-{[(4′-tert-butylbiphenyl-2- yl)carbonyl]amino}-3- (dimethylcarbamoyl)phenyl]acetoxy}methyl)-2- methyl-3-oxoisoindoline-1-carboxylate | 3.7 | 690 |
| | 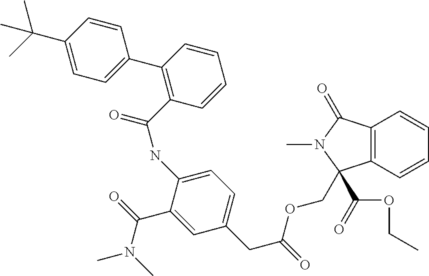 [SMILES: CCOC(=O)[C@]1(COC(=O)Cc2ccc(NC(=O)c3ccccc3-c3ccc(C(C)(C)C)cc3)c(C(=O)N(C)C)c2)c2ccccc2C(=O)N1C] | | |
| 67 | ethyl (1R)-1-({2-[4-{[(4′-tert-butyl-6- methylbiphenyl-2-yl)carbonyl]amino}-3- (dimethylcarbamoyl)phenyl]acetoxy}methyl)-2- methyl-3-oxoisoindoline-1-carboxylate | 3.8 | 704 |
| | 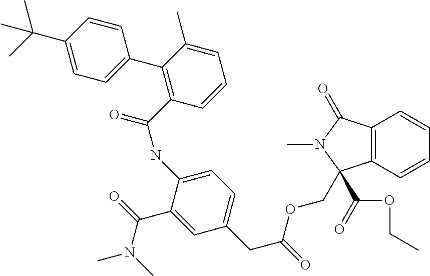 [SMILES: CCOC(=O)[C@]1(COC(=O)Cc2ccc(NC(=O)c3cccc(C)c3-c3ccc(C(C)(C)C)cc3)c(C(=O)N(C)C)c2)c2ccccc2C(=O)N1C] | | |
| 68 | ethyl (1R)-1-({2-[4-{[(4′-tert-butyl-5- methylbiphenyl-2-yl)carbonyl]amino}-3- (dimethylcarbamoyl)phenyl]acetoxy}methyl)-2- methyl-3-oxoisoindoline-1-carboxylate | 3.8 | 704 |
| | 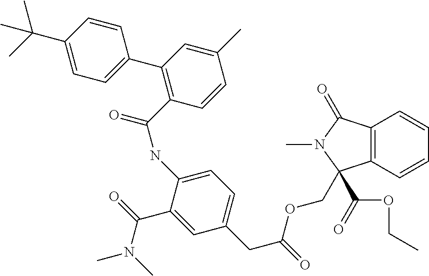 [SMILES: CCOC(=O)[C@]1(COC(=O)Cc2ccc(NC(=O)c3ccc(C)cc3-c3ccc(C(C)(C)C)cc3)c(C(=O)N(C)C)c2)c2ccccc2C(=O)N1C] | | |
| 69 | ethyl (1R)-1-({2-[4-{[(4′-tert-butyl-5- methoxybiphenyl-2-yl)carbonyl]amino}-3- (dimethylcarbamoyl)phenyl]acetoxy}methyl)-2- methyl-3-oxoisoindoline-1-carboxylate | 3.7 | 720 |
| | 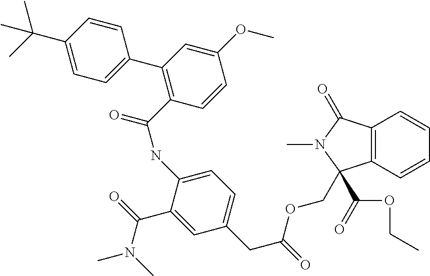 [SMILES: CCOC(=O)[C@]1(COC(=O)Cc2ccc(NC(=O)c3ccc(OC)cc3-c3ccc(C(C)(C)C)cc3)c(C(=O)N(C)C)c2)c2ccccc2C(=O)N1C] | | |
| 70 | ethyl (1R)-1-({2-[3-(dimethylcarbamoyl)-4-({[5- methoxy-4′-(trifluoromethyl)biphenyl-2- yl]carbonyl}amino)phenyl]acetoxy}methyl)-2- methyl-3-oxoisoindoline-1-carboxylate | 3.5 | 732 |
| | 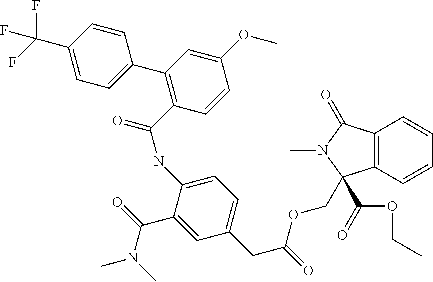 [SMILES: CCOC(=O)[C@]1(COC(=O)Cc2ccc(NC(=O)c3ccc(OC)cc3-c3ccc(C(F)(F)F)cc3)c(C(=O)N(C)C)c2)c2ccccc2C(=O)N1C] | | |
| 71 | ethyl (1R)-1-({2-[3-(dimethylcarbamoyl)-4-({[6- methoxy-4′-(trifluoromethyl)biphenyl-2- yl]carbonyl}amino)phenyl]acetoxy}methyl)-2- methyl-3-oxoisoindoline-1-carboxylate | 3.4 | 732 |
| | 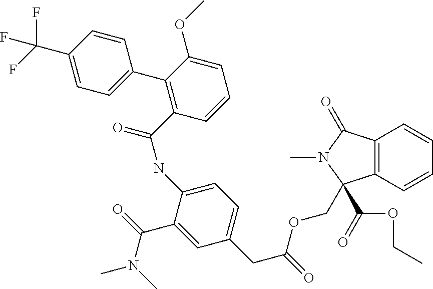 [SMILES: CCOC(=O)[C@]1(COC(=O)Cc2ccc(NC(=O)c3cccc(OC)c3-c3ccc(C(F)(F)F)cc3)c(C(=O)N(C)C)c2)c2ccccc2C(=O)N1C] | | |
| 72 | ethyl (1R)-1-({2-[3-(dimethylcarbamoyl)-4-{[(4′- isopropoxy-5-methylbiphenyl-2- yl)carbonyl]amino}phenyl]acetoxy}methyl)-2- methyl-3-oxoisoindoline-1-carboxylate | 3.7 | 706 |
| | 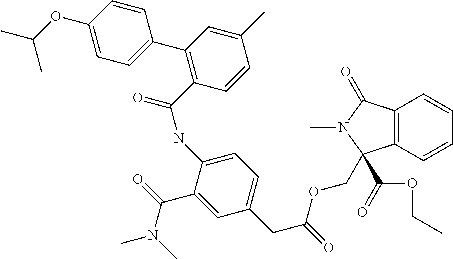 [SMILES: CCOC(=O)[C@]1(COC(=O)Cc2ccc(NC(=O)c3ccc(C)cc3-c3ccc(OC(C)C)cc3)c(C(=O)N(C)C)c2)c2ccccc2C(=O)N1C] | | |
| 73 | ethyl (1R)-1-({2-[3-(dimethylcarbamoyl)-4-{[(4′- isopropylbiphenyl-2- yl)carbonyl]amino}phenyl]acetoxy}methyl)-2- methyl-3-oxoisoindoline-1-carboxylate | 3.6 | 676 |
| | 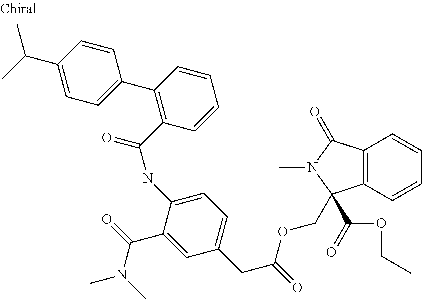 [SMILES: CCOC(=O)[C@]1(COC(=O)Cc2ccc(NC(=O)c3ccccc3-c3ccc(C(C)C)cc3)c(C(=O)N(C)C)c2)c2ccccc2C(=O)N1C] | | |
| 74 | ethyl (1R)-1-[(2-{4-[(biphenyl-2- ylcarbonyl)amino]-3- (dimethylcarbamoyl)phenyl}acetoxy)methyl]-2- methyl-3-oxoisoindoline-1-carboxylate | 3.2 | 634 |
| | 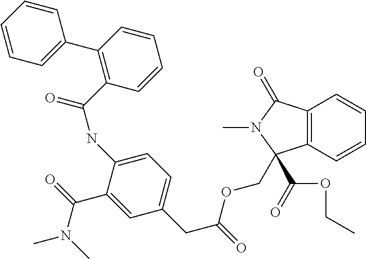 [SMILES: CCOC(=O)[C@]1(COC(=O)Cc2ccc(NC(=O)c3ccccc3-c3ccccc3)c(C(=O)N(C)C)c2)c2ccccc2C(=O)N1C] | | |
| 75 | ethyl (1R)-1-({2-[3-(dimethylcarbamoyl)-4-{[(2′- methoxybiphenyl-2- yl)carbonyl]amino}phenyl]acetoxy}methyl)-2- methyl-3-oxoisoindoline-1-carboxylate | 3.2 | 664 |
| | 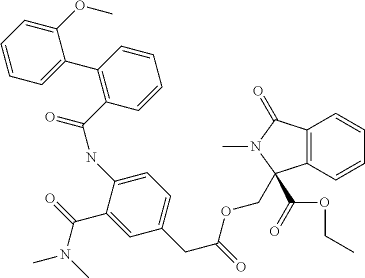 [SMILES: CCOC(=O)[C@]1(COC(=O)Cc2ccc(NC(=O)c3ccccc3-c3ccccc3OC)c(C(=O)N(C)C)c2)c2ccccc2C(=O)N1C] | | |

### Example 76

### Ethyl (1R)-1-({2-[3-(dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate

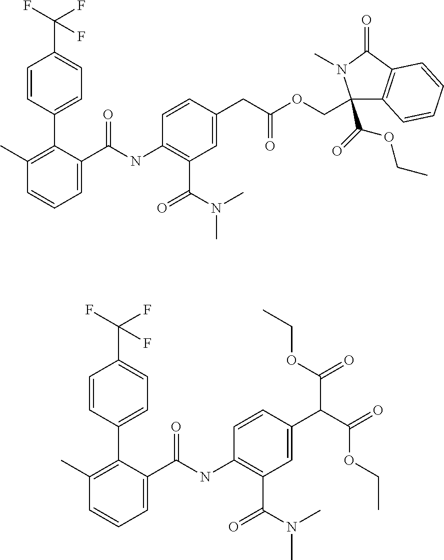 [SMILES: CCOC(=O)[C@]1(COC(=O)Cc2ccc(NC(=O)c3cccc(C)c3-c3ccc(C(F)(F)F)cc3)c(C(=O)N(C)C)c2)c2ccccc2C(=O)N1C ; CCOC(=O)C(C(=O)OCC)c1ccc(NC(=O)c2cccc(C)c2-c2ccc(C(F)(F)F)cc2)c(C(=O)N(C)C)c1]

### Step A: Diethyl [3-(dimethylcarbamoyl)-4-({[6-methyl-4′-(trifluoromethyl)biphenyl-2-yl]carbonyl}amino)phenyl]malonate

To a solution of 6-methyl-4′-(trifluoromethyl)biphenyl-2-carboxylic acid (30.0 g, 107 mmol) in 2-MeTHF (300. mL) was added oxalyl chloride (11.1 mL, 128 mmol) followed by DMF (0.05 mL, 0.6 mmol). Gas evolution was observed and the solids dissolved over time. The reaction mixture was stirred at room temperature for 1.5 hours. The reaction mixture was concentrated to give an oil. The oil was re-dissolved in 2-MeTHF (160 mL) and added dropwise to a solution of diethyl 2-(4-amino-3-(dimethylcarbamoyl)phenyl)malonate (34.5 g, 107 mmol) and DIEA (56.0 mL, 321 mmol) in 2-MeTHF (345 mL). The reaction mixture was stirred at room temperature for 0.5 hours, after which it was poured into water (510 mL). The 2-MeTHF layer was isolated, washed with sat. aq. NaHCO₃ (510 mL) and then carried into the next step.

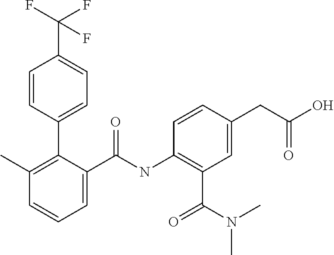 [SMILES: Cc1cccc(C(=O)Nc2ccc(CC(=O)O)cc2C(=O)N(C)C)c1-c1ccc(C(F)(F)F)cc1]

### Step B: [3-(Dimethylcarbamoyl)-4-({[6-methyl-4′-(trifluoromethyl)biphenyl-2-yl]carbonyl}amino)phenyl]acetic acid

To a solution of diethyl [3-(dimethylcarbamoyl)-4-({[6-methyl-4′-(trifluoromethyl)biphenyl-2-yl]carbonyl}amino)phenyl]malonate (21.8 g, 37.3 mmol) in 2-MeTHF (166 mL) was added 1 M aq. K₂CO₃ (166 mL) and EtOH (83 mL) at room temperature. The reaction mixture was heated to reflux for 50 hours and then cooled to room temperature. The reaction mixture contained two phases; the organic layer was evaporated to low volume, and 2-MeTHF (100 mL) was added. The 2-MeTHF was stripped down to low volume and this procedure was repeated with additional 2-MeTHF (100 mL). The residue was then partitioned between 2-MeTHF (70 mL) and 1 M aq. NaOH (70 mL). The aqueous layer was acidified with 2 M aq. HCl to pH=1. The resulting solid was collected by filtration and rinsed with water. Toluene (200 mL) was then added to this solid, and this mixture was heated to reflux until all solids dissolved. Toluene and water were collected using a Dean Stark apparatus until only about 11.5 mL of toluene remained. The solution was cooled to room temperature and the resulting white solid (15.5 g, 86% yield) was collected by filtration, rinsed with toluene, and dried under vacuum. ¹H NMR (400 MHz, CDCl₃) δ 2.12 (s, 3H) 2.90 (br. s., 3H) 3.08 (br. s., 3H) 3.51 (s, 2H) 7.09 (s, 1H) 7.18 (d, J=8.39 Hz, 1H) 7.31-7.51 (m, 5H) 7.61 (d, J=8.00 Hz, 2H) 7.96 (d, J=8.39 Hz, 1H) 8.97 (s, 1H).

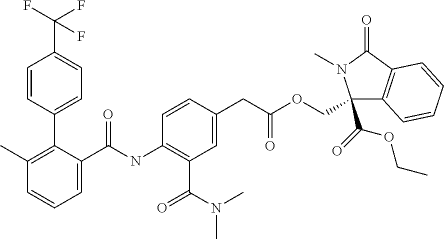 [SMILES: CCOC(=O)[C@]1(COC(=O)Cc2ccc(NC(=O)c3cccc(C)c3-c3ccc(C(F)(F)F)cc3)c(C(=O)N(C)C)c2)c2ccccc2C(=O)N1C]

### Step C: Ethyl (1R)-1-({2-[3-(Dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate

To a solution of [3-(dimethylcarbamoyl)-4-({[6-methyl-4′-(trifluoromethyl)biphenyl-2-yl]carbonyl}amino)phenyl]acetic acid (9.70 g, 20.0 mmol) in 2-MeTHF (100. mL) was added ethyl (1R)-1-(hydroxymethyl)-2-methyl-3-oxoisoindoline-1-carboxylate (4.99 g, 20.0 mmol) and DMAP (0.50 g, 4.08 mmol). After stirring for 5 min., 1-ethyl-3-(3-dimethylaminopropyl)carbodiimide (3.88 g, 25.0 mmol) was added. The reaction mixture was stirred at room temperature for 5 hours, after which the reaction mixture was poured into 1 M aq. HCl (100 mL). The organic layer was isolated, washed with sat. aq. NaHCO₃ and concentrated to dryness. The residue was purified by silica gel chromatography (gradient from pure heptane to 9:1 EtOAc/heptane) to afford the title compound (15.8 g, 83% yield). LCMS (ESI) m/z: 716.4 [M+H] (100%). ¹H NMR (400 MHz, CDCl₃) δ 1.19 (t, J=7.1 Hz, 3H), 2.12 (s, 3H), 2.90 (br. s., 3H), 3.08 (s, 3H), 3.11 (br. s., 3H), 3.30-3.41 (m, 2H), 4.11 (dq, J=10.7, 7.1 Hz, 1H), 4.22 (dq, J=10.7, 7.1 Hz, 1H), 4.67 (d, J=11.9 Hz, 1H), 4.81 (d, J=11.9 Hz, 1H), 6.90-6.97 (m, 2H), 7.34-7.40 (m, 2H), 7.42-7.51 (m, 6H), 7.61 (d, J=8.0 Hz, 2H), 7.79-7.83 (m, 1H), 8.06 (d, J=8.4 Hz, 1H), 9.07 (s, 1H).

### Example 77

### Ethyl (1R)-1-({2-[3-(dimethylcarbamoyl)-4-{[(4′-isopropoxybiphenyl-2-yl)carbonyl]amino}phenyl]acetoxy}methyl)-2-methyl-3-oxoisoindoline-1-carboxylate

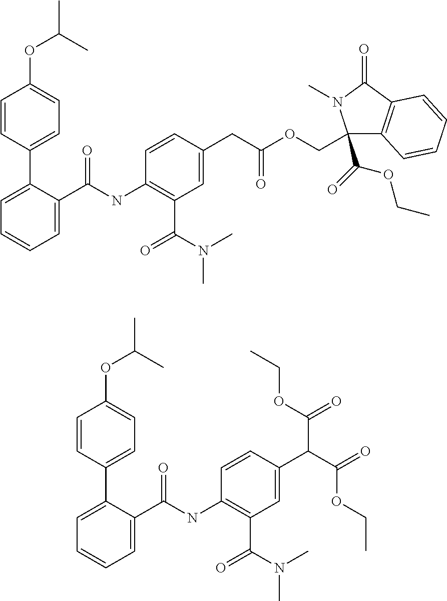 [SMILES: CCOC(=O)[C@]1(COC(=O)Cc2ccc(NC(=O)c3ccccc3-c3ccc(OC(C)C)cc3)c(C(=O)N(C)C)c2)c2ccccc2C(=O)N1C ; CCOC(=O)C(C(=O)OCC)c1ccc(NC(=O)c2ccccc2-c2ccc(OC(C)C)cc2)c(C(=O)N(C)C)c1]

### Step A: Diethyl [3-(Dimethylcarbamoyl)-4-{[(4′-isopropoxybiphenyl-2-yl)carbonyl]amino}phenyl]malonate

DMF (0.11 mL, 1.4 mmol) was added to a mixture of 4′-isopropoxybiphenyl-2-carboxylic acid (36.5 g, 142 mmol) and oxalyl chloride (13.6 mL, 157 mmol) in 2-MeTHF (365 mL). Gas evolution was observed within the first 20 minutes of the reaction. After 30 minutes of reaction time, the volatile components of the reaction mixture were removed by rotary evaporation. The resulting material was re-dissolved in 2-MeTHF (365 mL) and again concentrated by rotary evaporation. This product was again dissolved in 2-MeTHF (365 mL). To this solution was added solid diethyl 2-(4-amino-3-(dimethylcarbamoyl)phenyl)malonate (45.9 g, 142 mmol) and DIEA (37.3 mL, 214 mmol). The reaction mixture was stirred at room temperature for 1.5 hours, after which it was quenched with 1 M aq. HCl (400 mL), stirring for 10 min. The aqueous layer was isolated and extracted again with 2-MeTHF (100 mL). The combined organic layers were washed with water (400 mL) and concentrated to dryness. The obtained crude residue (83.5 g, >100% yield) was carried directly into the next step.

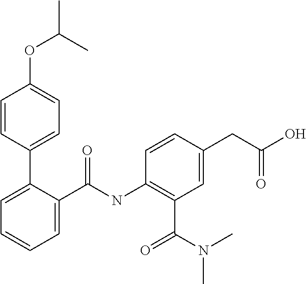 [SMILES: CC(C)Oc1ccc(-c2ccccc2C(=O)Nc2ccc(CC(=O)O)cc2C(=O)N(C)C)cc1]

### Step B: [3-(dimethylcarbamoyl)-4-{[(4′-isopropoxybiphenyl-2-yl)carbonyl]amino}phenyl]acetic acid

To a solution of diethyl [3-(dimethylcarbamoyl)-4-{[(4′-isopropoxybiphenyl-2-yl)carbonyl]amino}phenyl]malonate (79.8 g, 142 mmol) in THF (399 mL) was added 1 M aq. K₂CO₃ (399 mL) at room temperature. The reaction mixture was heated to reflux, and then MeOH (200. mL) was added. After stirring at reflux for 7 hours, the reaction mixture was cooled to room temperature. The aqueous layer was isolated and extracted once with each of MTBE (400 mL) and EtOAc (400 mL). The aqueous layer was then acidified to pH=2 using 2 M aq. HCl; this caused a sticky gum to form. This mixture was extracted with EtOAc (400 mL). The organic layer was concentrated to dryness affording a solid. Toluene (200 mL) was added to the solid, and the resulting mixture was heated to reflux. The solution was cooled to room temperature and a grey solid (60.2 g, 92% yield) was collected by filtration, rinsed with toluene, and dried under vacuum. ¹H NMR (400 MHz, CD₃OD) δ 1.29 (d, J=6.05 Hz, 6H), 2.89 (s, 3H), 2.98 (s, 3H), 3.60 (s, 2H), 4.60 (spt, J=6.05 Hz, 1H), 6.92 (d, J=8.58 Hz, 2H), 7.21 (d, J=1.56 Hz, 1H), 7.27-7.33 (m, 1H), 7.34-7.40 (m, 3H), 7.40-7.46 (m, 2H), 7.49-7.55 (m, 1H), 7.55-7.61 (m, 2H).

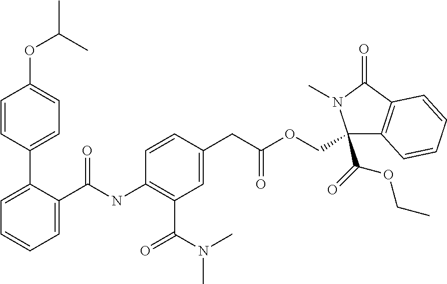 [SMILES: CCOC(=O)[C@]1(COC(=O)Cc2ccc(NC(=O)c3ccccc3-c3ccc(OC(C)C)cc3)c(C(=O)N(C)C)c2)c2ccccc2C(=O)N1C]

### Step C: Ethyl (1R)-1-({2-[3-(Dimethylcarbamoyl)-4-{[(4′-isopropoxybiphenyl-2-yl)carbonyl]amino}phenyl]acetoxy}methyl)-2-methyl-3-oxoisoindoline-1-carboxylate

To a solution of [3-(dimethylcarbamoyl)-4-{[(4′-isopropoxybiphenyl-2-yl)carbonyl]amino}phenyl]acetic acid (55.4 g, 120. mmol) in EtOAc (600. mL), was added ethyl (1R)-1-(hydroxymethyl)-2-methyl-3-oxoisoindoline-1-carboxylate (30.0 g, 120. mmol), and DMAP (3.0 g, 24.6 mmol). After stirring for 5 min., 1-ethyl-3-(3-dimethylaminopropyl)carbodiimide (23.3 g, 150. mmol) was added. The reaction mixture was stirred at room temperature for 23 hours, after which it was poured into 1 N HCl (100 mL). The organic layer was isolated, washed with sat. aq. NaHCO₃, and concentrated to dryness. The residue was purified by MPLC (gradient from pure heptane to 9:1 EtOAc/heptane) to afford the title compound (15.8 g, 83% yield). ¹H NMR (400 MHz, CDCl₃) δ 1.20 (t, J=7.1 Hz, 3H), 1.31 (d, J=6.0 Hz, 6H), 2.82 (br. s., 3H), 2.95 (br. s., 3H), 3.09 (s, 3H), 3.30-3.44 (m, 2H), 4.05-4.17 (m, 1H), 4.23 (dq, J=10.8, 7.2 Hz, 1H), 4.52 (dt, J=12.1, 6.0 Hz, 1H), 4.67 (d, J=11.9 Hz, 1H), 4.82 (d, J=11.9 Hz, 1H), 6.87 (d, J=8.4 Hz, 3H), 7.01 (dd, J=8.5, 1.7 Hz, 1H), 7.36-7.42 (m, 4H), 7.43-7.55 (m, 4H) 7.63-7.70 (m, 1H), 7.82 (dd, J=4.9, 2.9 Hz, 1H), 8.31 (d, J=8.4 Hz, 1H), 8.73 (s, 1H).

### PHARMACOLOGICAL DATA

### Assay for Human Liver and Intestinal Microsomal Stability

Pooled human liver or intestinal microsomes (final concentration 0.76 mg/mL) were diluted in 100 mM potassium phosphate buffer (pH 7.4) and preincubated at 37° C. The compounds of interest were dissolved in DMSO (30 mM), diluted to 100 μM in acetonitrile or methanol, and added to the microsomal incubations to achieve a final concentration of 1 μM with 1% or less final organic solvent. The mixtures were incubated for 30 min at 37° C. Aliquots were removed at predetermined times and quenched with an excess acetonitrile, containing an internal standard, on ice. Following centrifugation, the analytes were quantified by liquid chromatography-tandem mass spectrometry (LC-MS/MS). The compound remaining (%) was calculated from the analyte area ratio at the predetermined time/initial analyte area ratio at time 0×100.

Microsomal Assay Table

| | Human Liver Microsomes | | Human Intestinal Microsomes | |
|---|---|---|---|---|
| | Compound | Compound | Compound | Compound |
| | remaining | remaining | remaining | remaining |
| Example | (%) at 4 min | (%) at 30 min | (%) at 4 min | (%) at 30 min |
| 43 | 19 | 0.1 | 115 | 89 |
| 49 | 42 | 2.7 | 100 | 83 |
| 50 | 48 | 3.7 | 100 | 92 |
| 56 | 38 | 3 | 100 | 87 |
| 59 | 22 | 1.1 | 102 | — |
| 60 | 110 | 8.5 | 107 | 100 |
| 61 | 65 | 4.7 | 106 | 106 |
| 70 | 61 | 6.4 | 98 | 96 |
| 71 | 45 | 5.3 | 125 | 94 |
| 72 | 71 | 9.2 | 125 | 100 |

Inhibition of Apolipoprotein B Secretion from HepG2 Cells

To assess the inhibitory effect of the compounds of the invention on ApoB production, an endogenously ApoB producing cell line, HEPG2, was implemented in an in vitro ELISA assay. HEPG2 cells are plated at 25,000 per well (96-well plates) in media consisting of DMEM Low Glucose, 1% NEAA (non-essential amino acids), 10% FBS (heat inactivated), 1% L-Glutamine, and 1% Antimycotic-Antibiotic and incubated for 16-18 hours at 37° C. and 5% CO2. After the 16-18 hour incubation, a 12 point half-log serial of the inhibitor (1000 nM-0.003 nM) is prepared, and 1.5 ul of the serial dilution is added to HEPG2 cells, which have had their media decanted and replaced with 148 ul of fresh media, and the cells are incubated for 22-24 hours at 37° C. and 5% CO2. Two and a half hours before the 22-24 hour incubation period is completed, Nunc Maxisorp plates are coated with 100 ul of primary (coating) antibody (goat anti-apoB), diluting to a ratio of 1:1000 in 1% Carbonate-Bicarbonate Coating Buffer and incubated for at least 2 hours at room temperature before the primary antibody is decanted and the wells of the microplate are washed with 200 ul of washing buffer (1% PBS, 0.05% Tween20). Next, 200 ul of blocking buffer (1% PBS, 1:500 Western Block, 5 mg/ml BSA) is added to the wells of the microplate, and incubated for at least 30 mins. Following the 22-24 hours incubation period, cell plates are removed from the incubator, 20 ul of supernatant from each well is aspirated, added to a corresponding well in 96 well plate containing 140 ul of diluent (1:2-Blocking Buffer: Washing Buffer), and mixed. Then, 120 ul is aspirated from the dilutent-supernatant plate, added to the blocked Nunc Maxisorp 96 well plate, and placed at room temperature on a shaker to mix for at least 2 hours. The secondary (capture) antibody (mouse anti-human apoB) is prepared at a ratio of 1:4000, and the detection antibody (goat anti-mouse HRP conjugated antibody) at a ratio of 1:10,000. For each antibody, 100 ul is added to each well of the microplate and the microplate is incubated at room temperature for 2 hours, washing the microplates between each antibody addition. Colorimetric development is achieved by adding 50 ul of TMB reagent to each well, incubating for 5 mins at room temperature, then adding 50 ul of 2M Sulfuric Acid to each well. Absorbance at 450 nm is monitored in an Envision plate reader to measure the amount of ApoB formed.

Results reported as average IC50, low and high IC50 range (95% confidence interval).

ApoB Secretion Inhibition Table

| | | Average | Low | High |
|---|---|---|---|---|
| | Stereoisomer | IC50 | IC50 | IC50 |
| Example | Description | (nM) | (nM) | (nM) |
| 1 | Enantiomer | 0.495 | 0.352 | 0.696 |
| 2 | Enantiomer | 519 | 0.0111 | 24200000 |
| 3 | Enantiomer | 15.7 | 3.77 | 65.4 |
| 4 | Racemate | 0.846 | 0.547 | 1.31 |
| 6 | Enantiomer | 4.77 | 2.49 | 9.11 |
| 7 | Enantiomer | 15.6 | 7.73 | 31.6 |
| 8 | Racemate | 0.436 | 0.23 | 0.827 |
| 9 | Enantiomer | 0.2 | 0.125 | 0.319 |
| 10 | Enantiomer | 14.1 | 6.63 | 30.1 |
| 11 | Enantiomer | 29.4 | 9.76 | 88.6 |
| 12 | Enantiomer | 156 | 57 | 425 |
| 13 | Enantiomer | 7.18 | 5.01 | 10.3 |
| 14 | Racemate | 2.16 | 1.33 | 3.49 |
| 15 | Racemate | 1.45 | 0.954 | 2.2 |
| 16 | Racemate | 2.73 | 1.34 | 5.58 |
| 17 | Enantiomer | 3.07 | 1.28 | 7.38 |
| 18 | Enantiomer | 23.9 | 10.9 | 52.6 |
| 19 | Enantiomer | 0.107 | 0.0728 | 0.158 |
| 20 | Enantiomer | 1.76 | 0.739 | 4.2 |
| 21 | Racemate | 16.5 | 0.00134 | 204000 |
| 22 | Enantiomer | 83.5 | 3.74 | 1860 |
| 23 | Enantiomer | 0.333 | 0.0555 | 1.99 |
| 24 | Enantiomer | 28 | 15 | 52.5 |
| 25 | Enantiomer | 297 | 108 | 816 |
| 27 | Racemate | 30.8 | 0.00536 | 177000 |
| 28 | Racemate | 3.17 | 2.06 | 4.88 |
| 29 | Racemate | 2.04 | 1.28 | 3.25 |
| 30 | Racemate | 6.5 | 3.97 | 10.6 |
| 31 | Enantiomer | 10.1 | 6.22 | 16.4 |
| 32 | Enantiomer | 45.5 | 22.7 | 91.2 |
| 33 | Enantiomer | 6.5 | 2.86 | 14.8 |
| 34 | Enantiomer | 5.54 | 2.79 | 11 |
| 35 | Enantiomer | 0.46 | 0.221 | 0.957 |
| 36 | Enantiomer | 4.6 | 1.55 | 13.6 |
| 37 | Racemate | 8.47 | 5.99 | 12 |
| 38 | Enantiomer | 19.9 | 2.7 | 147 |
| 39 | Enantiomer | 2.34 | 1.59 | 3.44 |
| 40 | Enantiomer | 159 | 35.2 | 716 |
| 41 | Racemate | 0.701 | 0.449 | 1.1 |
| 42 | Racemate | 0.529 | 0.183 | 1.54 |
| 43 | Enantiomer | 1.72 | 1.13 | 2.63 |
| 44 | Racemate | 0.328 | 0.174 | 0.619 |
| 45 | Enantiomer | 1.63 | 0.888 | 3.01 |
| 46 | Enantiomer | 1.66 | 0.814 | 3.4 |
| 47 | Enantiomer | 7.56 | — | — |
| 48 | Enantiomer | 0.203 | 0.0817 | 0.503 |
| 49 | Enantiomer | 0.242 | 0.192 | 0.304 |
| 50 | Enantiomer | 0.124 | 0.0895 | 0.172 |
| 51 | Enantiomer | 0.159 | 0.1 | 0.253 |
| 53 | Enantiomer | 0.242 | 0.181 | 0.323 |
| 54 | Enantiomer | 0.124 | 0.0895 | 0.172 |
| 55 | Enantiomer | 0.154 | 0.0733 | 0.323 |
| 56 | Enantiomer | 0.147 | 0.109 | 0.198 |
| 57 | Enantiomer | 1.37 | 0.894 | 2.11 |
| 58 | Enantiomer | 7.18 | 3.6 | 14.3 |
| 59 | Enantiomer | 0.712 | 0.497 | 1.02 |
| 60 | Enantiomer | 0.535 | 0.346 | 0.828 |
| 61 | Enantiomer | 0.395 | 0.189 | 0.825 |
| 62 | Enantiomer | 1.17 | 0.676 | 2.03 |
| 63 | Enantiomer | 0.119 | 0.0526 | 0.269 |
| 64 | Enantiomer | 0.536 | 0.269 | 1.07 |
| 65 | Racemate | 0.38 | 0.174 | 0.831 |
| 66 | Enantiomer | 0.206 | 0.163 | 0.259 |
| 67 | Enantiomer | 0.244 | 0.171 | 0.348 |
| 68 | Enantiomer | 0.178 | 0.12 | 0.265 |
| 69 | Enantiomer | 0.18 | 0.0837 | 0.387 |
| 70 | Enantiomer | 0.414 | 0.242 | 0.709 |
| 71 | Enantiomer | 0.176 | 0.111 | 0.279 |
| 72 | Enantiomer | 0.119 | 0.0894 | 0.158 |
| 73 | Enantiomer | 0.162 | 0.11 | 0.238 |
| 74 | Enantiomer | 1.28 | 0.371 | 4.38 |
| 75 | Enantiomer | 4.16 | 1.91 | 9.07 |
| 76 | Enantiomer | 0.242 | 0.192 | 0.304 |
| 77 | Enantiomer | 0.119 | 0.0894 | 0.158 |

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having the Formula I 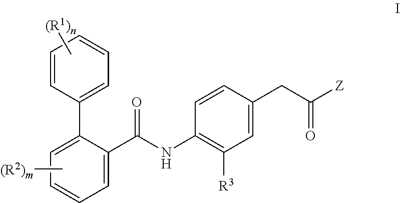
wherein:
R¹ is, (C₁-C₆)alkyl, (C₃-C₇)cycloalkyl, (C₁-C₆)alkoxy, —CN, —C(O)—OH, hydroxyl, or halo, wherein each alkyl and alkoxy is optionally substituted with one or more hydroxyl, halo or oxy, and n is 0, 1 or 2;
R² is (C₁-C₆)alkyl, (C₃-C₇)cycloalkyl, (C₁-C₆)alkoxy, —O(O)—OH, hydroxyl, or halo wherein each alkyl and alkoxy is optionally substituted with one or more hydroxyl, halo or oxy, and m is 0, 1 or 2;
R³ is hydrogen, (C₁-C₆)alkyl, (C₃-C₇)cycloalkyl, (C₁-C₆)alkoxy, —O(O)—OH, hydroxyl, halo, or —C(O)—N—R^{4a}R^{4b};
R^{4a} and R^{4b} are each independently hydrogen, (C₁-C₆)alkyl, (C₁-C₆)alkoxy, (C₃-C₇)cycloalkyl, or R^{4a} and R^{4b} are taken together with the N to which they are attached to form a 4- to 7-membered heterocycle optionally substituted with (C₁-C₆)alkyl;
Z is —O—R⁵;
R⁵ is 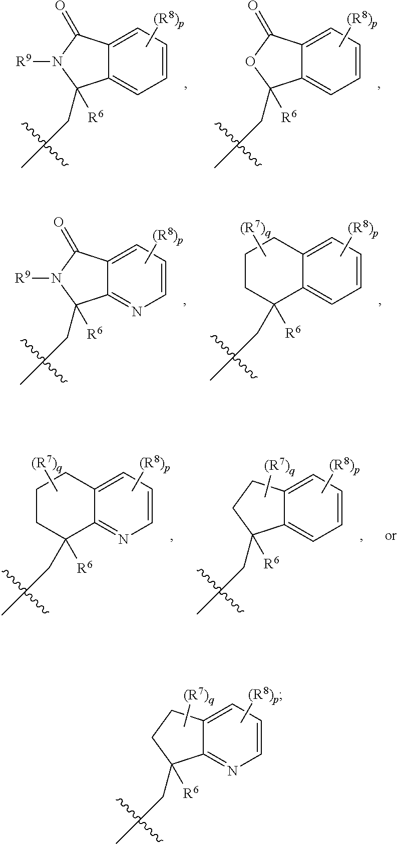
R⁶ is —C(O)—O—(C₁-C₆)alkyl, —C(O)—O—(C₁-C₆)alkyl-aryl, or —C(O)—OH;
R⁷ is hydrogen, hydroxyl, oxo, (C₁-C₆)alkyl, or (C₁-C₆)alkoxy, wherein each alkyl and alkoxy is optionally substituted with hydroxyl, halo or oxy, and q is 0, 1 or 2;
R⁸ is (C₁-C₆)alkyl, (C₃-C₇)cycloalkyl, (C₁-C₆)alkoxy, or halo, wherein each alkyl and alkoxy is optionally substituted with hydroxyl, halo or oxy, and p is 0, 1 or 2; and
R⁹ is hydrogen, (C₁-C₆)alkyl, (C₁-C₆)alkoxy, (C₃-C₇)cycloalkyl, aryl, or aralkyl wherein each alkyl and alkoxy is optionally substituted with hydroxyl, halo or oxy, and each aryl and aralkyl are optionally substituted with (C₁-C₆)alkyl, (C₁-C₆)alkoxy, hydroxyl, halo or oxy;
or a pharmaceutically acceptable salt thereof.

2. A compound according to claim 1 wherein R³ is —C(O)—N—R^{4a}R^{4b} and q is 0.

3. A compound according to claim 2 wherein R⁵ is

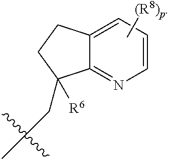

4. A compound according to claim 2 wherein R⁵ is

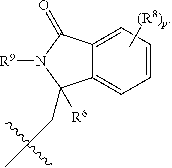

5. A compound according to claim 3 wherein p is 0 and R⁹ is hydrogen or (C₁-C₃)alkyl.

6. A compound according to claim 5 wherein R⁶ is —C(O)—O—(C₁-C₆)alkyl.

7. A compound according to claim 6 wherein m and n are each independently 0 or 1 and R¹ and R² are each independently (C₁-C₃)alkyl, (C₁-C₃)alkoxy or trifluoromethyl.

8. The compound:
Ethyl (1R)-1-({2-[3-(Dimethylcarbamoyl)-4-({[6-methyl-4′-(trifluoromethyl)biphenyl-2-yl]carbonyl}amino)phenyl]acetoxy}methyl)-2-methyl-3-oxoisoindoline-1-carboxylate;
Ethyl (1R)-1-({2-[3-(dimethylcarbamoyl)-4-{[(4′-isopropoxybiphenyl-2-yl)carbonyl]amino}phenyl]acetoxy}methyl)-2-methyl-3-oxoisoindoline-1-carboxylate;
Ethyl 1-({2-[3-(dimethylcarbamoyl)-4-({[5-methyl-4′-(trifluoromethyl)biphenyl-2-yl]carbonyl}amino)phenyl]acetoxy}methyl)-2-methyl-3-oxoisoindoline-1-carboxylate;
Ethyl 7-({2-[3-(dimethylcarbamoyl)-4-({[5-methyl-4′-(trifluoromethyl)biphenyl-2-yl]carbonyl}amino)phenyl]acetoxy}methyl)-6,7-dihydro-5H-cyclopenta[b]pyridine-7-carboxylate;
Ethyl 7-({2-[3-(dimethylcarbamoyl)-4-({[6-methyl-4′-(trifluoromethyl)biphenyl-2-yl]carbonyl}amino)phenyl]acetoxy}methyl)-6,7-dihydro-5H-cyclopenta[b]pyridine-7-carboxylate:
Ethyl (1R)-1-({2-[3-(dimethylcarbamoyl)-4-({[5-methoxy-4′-(trifluoromethyl)biphenyl-2-yl]carbonyl}amino)phenyl]acetoxy}methyl)-2-methyl-3-oxoisoindoline-1-carboxylate;
Ethyl (1R)-1-({2-[3-(dimethylcarbamoyl)-4-({[6-methoxy-4′-(trifluoromethyl)biphenyl-2-yl]carbonyl}amino)phenyl]acetoxy}methyl)-2-methyl-3-oxoisoindoline-1-carboxylate;
Ethyl (1R)-1-({2-[3-(dimethylcarbamoyl)-4-{[(4′-isopropoxy-5-methylbiphenyl-2-yl)carbonyl]amino}phenyl]acetoxy}methyl)-2-methyl-3-oxoisoindoline-1-carboxylate;
Ethyl 7-({2-[3-(dimethylcarbamoyl)-4-{[(4′-isopropoxybiphenyl-2-yl)carbonyl]amino}phenyl]acetoxy}methyl)-6,7-dihydro-5H-cyclopenta[b]pyridine-7-carboxylate; or
Ethyl 7-({2-[3-(dimethylcarbamoyl)-4-({[6-methoxy-4′-(trifluoromethyl)biphenyl-2-yl]carbonyl}amino)phenyl]acetoxy}methyl)-6,7-dihydro-5H-cyclopenta[b]pyridine-7-carboxylate
or a pharmaceutically acceptable salt thereof.

9. The compound:
[3-dimethylcarbamoyl-4-{[(6-methyl-4′-trifluoromethylbiphenyl-2-yl)carbonyl]amino}phenyl]acetate;
[3-dimethylcarbamoyl-4-{[(4′-isopropoxybiphenyl-2-yl)carbonyl]amino}phenyl]acetate;
[3-dimethylcarbamoyl-4-{[(5-methyl-4′-trifluoromethylbiphenyl-2-yl)carbonyl]amino}phenyl]acetate;
[3-dimethylcarbamoyl-4-{[(5-methoxy-4′-trifluoromethylbiphenyl-2-yl)carbonyl]amino}phenyl]acetate;
[3-dimethylcarbamoyl-4-{[(6-methoxy-4′-trifluoromethylbiphenyl-2-yl)carbonyl]amino}phenyl]acetate;
[3-dimethylcarbamoyl-4-{[(5-methyl-4′-isopropoxybiphenyl-2-yl)carbonyl]amino}phenyl]acetate; or
[3-dimethylcarbamoyl-4-{[(6-methoxy-4′-trifluoromethylbiphenyl-2-yl)carbonyl]amino}phenyl]acetate;
or a pharmaceutically acceptable salt thereof.

10. A pharmaceutical composition comprising a compound according to claim 1, present in a therapeutically effective amount, in an admixture with at least one pharmaceutically acceptable excipient.

11. The composition of claim 10 further comprising at least one additional pharmaceutical agent selected from the group consisting of an anti-obesity agent, an anti-diabetic agent, an anti-hyperglycemic agent, a lipid lowering agent, and an anti-hypertensive agent.

12. A method for the treatment of diabetes or obesity comprising the administration of a therapeutically effective amount of compound or pharmaceutically acceptable salt of claim 1, to a patient in need thereof.

13. A method for treating a metabolic or metabolic-related disease, condition or disorder comprising the step of administering to a patient a therapeutically effective amount of a compound or a pharmaceutically acceptable salt thereof, of claim 1.

14. A method of treating a disease, condition or disorder that inhibits microsomal triglyceride transfer protein (MTP) and/or apolipoprotein B (Apo B) secretion, comprising the administration of a therapeutically effective amount of a compound or a pharmaceutically acceptable salt thereof, according to claim 1 to a patient in need thereof.

15. The method of claim 14, wherein the disease, condition or disorder is selected from the group consisting of Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, pancreatitis, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance, hyper apo B lipoproteinemia, Alzheimer's disease, schizophrenia, impaired cognition, inflammatory bowel disease, ulcerative colitis, Crohn's disease, and irritable bowel syndrome.

16. The method of claim 14, comprising the step of administering to a patient in need of such treatment two separate pharmaceutical compositions comprising
(i) a first composition according to claim 10; and,
(ii) a second composition comprising at least one additional pharmaceutical agent selected from the group consisting of an anti-obesity agent, an anti-diabetic agent, an anti-hyperglycemic agent, a lipid lowering agent, and an anti-hypertensive agent, and
(iii) at least one pharmaceutically acceptable excipient.

17. The method of claim 16, wherein said first composition and second composition are administered simultaneously.

18. The method of claim 17, wherein said first composition and said second composition are administered sequentially and in any order.

19. The method according to claim 14, wherein the disease, condition or disorder is selected from the group consisting of diabetic retinopathy, macular degeneration, and cataract.
